SEQUENCE LISTING (16 sequences)

SEQ ID NO: 1 - protein, 545 aa, synthetic construct
MKMVVDTIATDRCVYRSKLPDIEIKNDMSLHNYCFQNIGAYRDNPCLINGSTGEVYTYGEVETTARRVAAGLHRMGVQQREVIMILLPNSPEFVFAFLGASFRGAMSTTANPFYTPQEIAKQVKASGAKLIVTMSAYVDKVRDLAEERGVKVVCVDAPPPGCSHFSELSGADESELPEVDIDPDDVVALPYSSGTTGLPKGVMLTHRSQVTSVAQQVDGENPNLYFRPDDVLLCVLPLFHIYSLNSVLFCGLRVGAAILIMQKFEITALMELVQKYKVTIAPIVPPIVLAIAKSPLVDKYDLSSIRTVMSGAAPMGKELEDAVRAKLPNAKLGQGYGMTEAGPVLSMCLAFAKEPFEIKSGSCGTVVRNAQLKIVDPETGAYLPRNQPGEICIRGSQIMKGYLNDAAATQRTIDKEGWLHTGDIGYVDDDEELFIVDRLKEIIKYKGFQVAPAELEAILITHPNIADAAVVPMKDEAAGEVPVAFVVTSNGSVISEDEIKQFISKQVVFYKRINRVFFVDSIPKAPSGKILRKDLRGRLAAGIPK

SEQ ID NO: 2 - protein, 397 aa, synthetic construct
MSKTVEDRAAQRAKGPATVLAIGTATPANVVYQTDYPDYYFRVTKSEHMTKLKNKFQRMCDRSTIKKRYMVLTEELLEKNLSLCTYMEPSLDARQDILVPEVPKLGKEAADEAIAEWGRPKSEITHLIFCTTCGVDMPGADYQLTKLLGLRSSVRRTMLYQQGCFGGGTVLRLAKDLAENNAGARVLVVCSEITTAVNFRGPSDTHLDLLVGLALFGDGAAAVIVGADPDPTLERPLFQIVSGAQTILPDSEGAINGHLREVGLTIRLLKDVPGLVSMNIEKCLMEAFAPMGIHDWNSIFWIAHPGGPTILDQVEAKLGLKEEKLKSTRAVLREYGNMSSACVLFILDEVRKRSMEEGKTTTGEGFDWGVLFGFGPGFTVETVVLHSMPIPKADEGR

SEQ ID NO: 3 - protein, 394 aa, synthetic construct
MSKMVEEHWAAQRARGPATVLAIGTANPPNVLYQADYPDFYFRVTKSEHMTQLKEKFKRICDKSAIRKRHLHLTEELLEKNPNICAHMAPSLDARQDIAVVEVPKLAKEAATKAIKEWGRPKSDITHLIFCTTCGVDMPGADYQLTTLLGLRPTVRRTMLYQQGCFAGGTVLRHAKDFAENNRGARVLAVCSEFTVMNFSGPSEAHLDSMVGMALFGDGASAVIVGADPDFAIERPLFQLVSTTQTIVPDSDGAIKCHLKEVGLTLHLVKNVPDLISNNMDKILEEAFAPLGIRDWNSIFWTAHPGGAAILDQLEAKLGLNKEKLKTTRTVLREYGNMSSACVCFVLDEMRRSSLEEGKTTSGEGLEWGILLGFGPGLTVETVVLRSVPISTAN

SEQ ID NO: 4 - protein, 394 aa, synthetic construct
MSKTVEEIWAAQRARGPATVLAIGTAAPANVVYQADYPDYYFRITKSEHMTELKEKFRRMCDKSMITKRHMHLSEELLKNNPDICAYMAPSLDARQDMVVVEVPKLGKEAAAKAIKEWGRPKSAITHLIFCTTSGVDMPGADFQLTKLLGLCPSVRRTMLYQQGCFAGGTVLRLAKDLAENNAGARVLVVCSEITAVTFRGPSETHLDSMVGQALFGDGASAIIVGADPDPVIERPLFQIVSAAQTILPDSDGAIDGHLREVGLTFHLLKDVPGLISKNIEKSLKEAFAPLGIDDWNSIFWIVHPGGPAILDQVEAKLRLKVEKLKTTRTVLSEYGNMSSACVLFILDEMRRNSMEEGKATTGEGLHWGVLFGFGPGLTVETVVLHSLPIAEAN

SEQ ID NO: 5 - protein, 361 aa, synthetic construct
MEQAVFKDQSPSRDDIDEELFQSALYLSTAVVTVPAAIMAANDLDVLQIIAKAGPGAHLSPTEIVSHLPTRNPNAAAALHRILRVLASHSILECSSRCEGEAKYGLRPVCKFFLNDKDGVSLNAMPSFVQSRVFIDSWQYMKDAVLEGVVPFEKAYGMPFYQFQAVNTKFKETFAKAMAAHSTLVVKKMLDTYNGFEGLTELMDVAGGTGSTLNLIVSKYPQIKGTNFDLKHVIEAAPNYPGVKHLSGDMFDSIPSAKNIIMKWILHNWSDEHCVKLLKNCYTSLPEFGKLIVVDSIVGEDVDAGLTTTNVFGCDFTMLTFFPNAKERTREEFQDLAKASGFSTFKPICCAYGVWVMEFHK

SEQ ID NO: 6 - protein, 360 aa, synthetic construct
MNDQELHGYSQNAQPQLWNLLLSFINSMSLKCAVELGIPDIIHSHAQTPINITDLAASIPIPPNKTSQFRRLMRLLVHSNVFSVHKREDGDEGFLLTPMSRILVTSNDNNGGNLSPFVSMMVDPSLVSPWHFLGQWLKGNDTQGTPFRMCHGEEMWDWANKYPDFNKKFNMAMVCDSQYLMKIIVKKCATAFEGKRSLIDVGGGTGGAARSIAEAFPDIQEVSVLDLPHVVAGLPNDSRVKFVGGDMFHTIPPADVVLLKAIFHGWNDEECIKILKNCKKAIPSKEEGGKVMILDMVVNSAPGDHMITEDQYFMDLMMITYARGLERDENEWKKLFKDAGFTSYKITHGLGTSSLIELYP

SEQ ID NO: 7 - protein, 511 aa, synthetic construct
MEQAQWVDPTLLPAFVGIIFFFLGMFFGRSSLGAGKGAAPRSTSSTEWPDGPPKLPIIGNLHQLNKGGELVHHNLAKLAQSYDRAMTIWVGSWGPMIVVSDADLAWEVLVTKSPDFAGRVLSKLSHLFNANYNTVVAYDAGPQWQSLRRGLQHGPLGPAHVSAQARFHEEDMKLLVSDMMRAAQKGGSNGVVEPLAYVRRATIRFLSRLCFGEAFNDEAFVEGMDEAVEETIGATGHARILDAFYFTRHLPIIRRSFIDTVNAKKKIESLVRPLLSRPAPPGSYLHFLLSTDAPENMIIFRIFEVYLLGVDSTASTTTWALAFLVSNQQAQEKLHNELAQYCASQNNQIIKADDVGKLSYLLGVVKETMRMKPIAPLAVPHKTLKETMLDGKRVAAGTTVVVNLYAVHYNPKLWPEPEQFRPERFVVGASGGNGGGSSEYMLQSYLPFGGGMRSCAGMEVGKLQVAMVVANLVMAFKWLPEEEGKMPDLAEDMTFVLMMKKPLAAKIVPRA

SEQ ID NO: 8 - protein, 326 aa, synthetic construct
METERKSRICVTGAGGFVASWVVKLFLSKGYLVHGTVRDLGEEKTAHLRKLEGAYHNLQLFKADLLDYESLLGAITGCDGVLHVATPVPSSKTAYSGTELVKTAVNGTLNVLRACTEAKVKKVIYVSSTAAVLVNPNLPKDKIPDEDCWTDEEYCRTTPFFLNWYCIAKTAAEKNALEYGDKEGINVISICPSYIFGPMLQPTINSSNLELLRLMKGDDESIENKFLLMVDVRDVAEAILLLYEKQETSGRYISSPHGMRQSNLVEKLESLQPGYNYHKNFVDIKPSWTMISSEKLKKLGWKPRPLEDTISETVLCFEEHGLLENE

SEQ ID NO: 9 - DNA, 1638 bp, synthetic construct
  source 1..1638: mol_type = other DNA
atgaagatggtagtagacactattgctactgatcgatgtgtataccggtctaagctgccggacattgagatcaagaacgacatgtcgttgcacaattattgtttccagaacattggtgcttaccgggacaatccttgtctcatcaatggcagcaccggcgaggtgtacacgtacggcgaggtggagacgacggcgaggagggtggccgccgggctgcaccggatgggggtgcagcagcgggaggtgatcatgatcctcctccccaactcgccggagttcgtcttcgccttcctcggcgcctccttccgcggggccatgtccaccaccgccaaccccttctacacgccgcaggagatcgccaagcaggtcaaggcctccggcgcgaagctcatcgtcaccatgtccgcctacgtcgacaaggtcagggacctggccgaggagcgcggcgtcaaagtggtgtgcgtcgacgcgccgcccccggggtgctcccacttctccgagctgtccggcgccgacgagtcggagctgcccgaggtggatattgaccccgacgacgtggtggcgctgccatactcctccggcaccaccggcctccctaaaggagtgatgctcacacaccgcagccaggtgacgagcgttgcccagcaagtcgacggcgagaacccgaatctatacttccggccagacgacgtcctgctctgcgttcttcccctcttccacatctactccctcaactcggtgctcttctgcggcctgcgcgtcggggcggcgatcctcatcatgcagaagttcgagatcacggcgctgatggagctggtgcagaagtacaaggtgaccattgcgcccatcgttccgcccatcgttcttgccatcgccaagagcccgctcgtcgacaagtacgacttgtcgtccattcggacggtgatgtccggcgccgccccgatggggaaggagctcgaagacgccgtccgggccaagcttcccaacgccaagctcggccagggctatgggatgacggaggcagggccagtgctgtccatgtgtttggccttcgccaaggagcccttcgagatcaagtctggttcttgcggcaccgtggtcaggaacgcccagctcaagatcgtcgacccagaaaccggtgcctacctgcccagaaaccaacccggcgaaatttgcatccgaggctcccaaatcatgaaagggtatcttaatgacgcggcggctacgcagaggacgatcgacaaggaagggtggctgcacaccggcgacattggctatgtcgacgacgacgaggagctcttcattgtcgataggttgaaggagatcattaagtacaagggcttccaagtcgcccctgccgagctcgaagccattctcattactcaccctaacattgctgatgccgctgttgtcccgatgaaagatgaggcagcaggggaagtgccagtggcatttgtggtgacctccaatggatcagtcatcagtgaggatgagatcaagcagttcattagcaagcaggtggtgttctacaagcgaatcaatcgagtctttttcgttgattcaattcctaaagcaccctctgggaagattttgaggaaggatttgaggggaagattggcagctggtatacccaagtag

SEQ ID NO: 10 - DNA, 1194 bp, synthetic construct
  source 1..1194: mol_type = other DNA
atgtcgaagacggtggaggatcgggcagcgcagcgggcaaaggggccggcaacagtgctggccatcggcacggctacgccggccaatgtggtgtaccagaccgattatccggactactacttcagggtcaccaagagcgagcatatgaccaaactcaagaacaagtttcaacgcatgtgcgacaggtcgacgataaagaagaggtacatggttttgacagaggagctgctagagaagaatctgagtttgtgcacctacatggaaccctccctcgacgcccgccaagacattctcgtgccggaggtccccaagctcggcaaggaggccgccgacgaggccatcgccgaatggggacgccccaagtcggaaatcacccacctcatcttttgcactacctgcggcgtcgacatgcccggcgccgactaccagctcaccaagctcctcggtctccgctcctccgtccgtcgcaccatgctctatcagcagggatgctttggcggaggcaccgttctccgcctcgccaaggacctcgccgagaacaacgctggtgcccgcgtcctcgtcgtctgctccgagatcaccactgccgtcaacttccgagggccttccgacacccacctcgacttattggtcggcttagccctgttcggcgacggtgcggccgcggtcatagtcggtgcggatccagatcctaccctcgagcggccgctctttcaaatcgtatctggagcacagacgattctaccggactcggagggggccatcaacggccatctccgggaggtggggctaaccatccgcctactcaaggacgtacctgggcttgtgtcgatgaacattgagaagtgcctcatggaggcgtttgcaccgatgggcatccacgactggaactccatcttttggatagcccatcccggggggcccaccatactagaccaagtggaggccaagctgggtctaaaggaggagaagctcaagtcgacgagggctgttctgagggagtatggcaacatgtctagcgcctgtgtcttgttcatactggacgaggtaaggaagaggagcatggaggaggggaagacgacaaccggtgaggggttcgattggggagttctattcggctttgggcctggcttcacagtggagaccgtcgtcttgcacagcatgcccatccccaaagccgatgaaggcagataa

SEQ ID NO: 11 - DNA, 1185 bp, synthetic construct
  source 1..1185: mol_type = other DNA
atgtcgaagatggtggaggagcattgggcagcgcagcgggcgaggggaccggcgacagtgctggccatcggcactgcaaatcctcccaatgtgttgtaccaggcagattatcccgacttctactttagggtcaccaagagtgagcacatgacccagctaaaggagaagtttaaacgtatatgtgataagtcagcaataagaaagcgccacctccatctaaccgaggagctgctggagaagaaccctaacatatgtgcacacatggccccctccctcgacgcccggcaagacattgcggtggtggaggtccccaagctagccaaagaagctgcaaccaaggccatcaaggagtgggggcgacccaagtccgacatcacccacctcatcttctgcaccacctgcggcgtggacatgcccggcgccgactaccaactcaccacgctcctcggcctccgccccacggtccgccgcaccatgctctaccaacagggctgcttcgccggcggcacagtccttcgccatgccaaggacttcgccgagaacaataggggtgctcgtgtcctcgccgtctgctcggagttcaccgtcatgaacttcagcggaccgtcggaggcccacttagacagcatggtcggtatggcgctgttcggtgatggcgcctcggctgtcatcgtcggcgccgatcctgactttgccattgaacgaccgctctttcaactggtttctacaacacaaactattgtcccggactcggacggagccatcaagtgccatctcaaggaggtgggcctaaccctgcatctcgttaagaatgtaccagatctcatatcaaataacatggacaagatcctcgaagaggcatttgcaccattgggcatcagagattggaactcaatcttttggacagctcatccaggtggagcagccatactcgaccagttggaggccaagctcggtctgaacaaggagaagctcaagactacaagaacagttctgagggagtatggaaacatgtccagcgcctgtgtttgtttcgtcctggacgagatgaggagaagtagcttggaggaggggaagacaacgtccggggaagggttggaatggggaattctgctagggtttgggcctgggttgacagtggagacagtcgtcttgcgtagcgtacccatctcgacagccaattaa

SEQ ID NO: 12 - DNA, 1185 bp, synthetic construct
  source 1..1185: mol_type = other DNA
atgtcgaagaccgtagaggagatttgggcggcgcagcgggcgaggggaccagccacggtgctggccatcggcactgctgcgccggccaatgtggtgtaccaggccgattatccggactactactttaggatcaccaagagcgagcacatgacagagctcaaggagaagttccgacgaatgtgtgacaagtcgatgataacgaagcggcacatgcacttgtcggaggagctgttgaaaaacaaccctgacatctgtgcctacatggccccttccctcgacgcccgccaagatatggtcgtggtggaggtacccaagctcggcaaggaggcggccgccaaggccatcaaggaatggggccgcccaaagtcggccatcacccacctcatcttctgcaccacctccggcgtcgacatgcccggcgccgatttccagctcaccaagctactcggcctctgcccctccgttcgccgcaccatgctctaccagcagggctgcttcgccggcggtacggttctccgccttgccaaggacctcgccgagaacaatgcgggcgcgagggtcctcgtcgtctgctccgagatcaccgccgtcaccttccgcggcccctcggagactcacctcgatagcatggtcggccaggccctgttcggtgatggtgcctctgccatcatcgtcggtgccgaccccgaccccgtcatagaaaggccactctttcaaattgtatctgcggctcagaccatccttcccgactcggatggggcaatagacggccatctccgagaagtgggtctaaccttccacctcctcaaggacgtacctgggctcatctcaaagaacatcgagaagagcctaaaggaggcgtttgcaccgctgggcatcgacgactggaactcgatattttggattgttcatccaggcgggccggccattctagaccaggtggaggcgaagctgcgtctgaaagtggagaagctgaagacaacgagaacagttttgagtgagtacgggaatatgtcgagcgcttgcgtgttgttcatacttgacgagatgaggaggaacagcatggaagaagggaaggcgacgaccggtgaagggttacattggggagttttgtttggttttgggccgggcttgacagtggagacggtcgtcttgcatagtttgcccatcgccgaggccaactaa

SEQ ID NO: 13 - DNA, 1086 bp, synthetic construct
  source 1..1086: mol_type = other DNA
atggagcaagctgtgttcaaagaccaatccccaagcagggatgatattgatgaagagctctttcaatctgctctatatcttagcactgcggttgtcaccgtgccggcggcaatcatggctgcaaatgaccttgacgtgctgcagataattgccaaagctggcccaggtgctcacctatctccgacagagattgtcagccaccttcccacccgtaaccctaatgccgcggcggcgcttcaccggatactccgagtactagccagccactccatccttgaatgctcgtcgagatgcgagggcgaggcaaaatatggattaaggccggtgtgcaagttctttctcaatgataaggatggtgtctccttgaatgccatgccatccttcgttcaaagtagagtttttatagatagctggcaatatatgaaagatgctgttcttgagggggtagtcccctttgagaaagcctatggtatgcctttttatcagtttcaagcagtgaacaccaaattcaaagaaaccttcgccaaagccatggctgctcactcaactttggtagtaaaaaagatgcttgacacatacaatgggtttgagggactcactgagttgatggatgttgctggtggaaccggttccaccctcaacctcattgtctccaaatacccacaaatcaaaggcacaaactttgatctcaaacatgtcattgaggccgcaccaaactaccctggggtgaagcatttgagtggggacatgtttgatagcattccaagtgcaaagaacattattatgaagtggatactacataattggagcgacgagcactgtgtaaaactcctcaagaactgctacacttccttaccagaatttgggaagttgattgtggttgattccattgtgggtgaggatgttgatgctggtttgacgacaacaaatgtctttggatgcgacttcacaatgctaactttcttccccaatgcaaaagagaggacccgtgaagaattccaagacctggccaaagctagtggcttctcaacgttcaaaccgatctgctgcgcctatggcgtgtgggttatggaatttcacaaataa

SEQ ID NO: 14 - DNA, 1083 bp, synthetic construct
  source 1..1083: mol_type = other DNA
atgaatgatcaagagttgcatggatactcacaaaatgctcaacctcagctatggaacctcctgttgagcttcataaattccatgtcccttaagtgtgcagtggagttgggcatccccgatataatacatagccatgcccaaacaccaatcaacataaccgaccttgctgcctccatacccattcccccaaacaaaacaagccaattccgccgactcatgcgcctcctggttcactccaacgtcttttccgtccataaacgtgaggatggtgatgaggggttcctcctaactcctatgtccaggatccttgtcacgtcgaacgacaataatggaggtaacttgtcaccctttgtttccatgatggttgatccgtccctggtgtctccatggcacttccttggtcaatggctcaaaggcaatgacacccaaggcacaccatttcgcatgtgccatggtgaagaaatgtgggactgggccaacaagtacccggacttcaacaagaagttcaacatggcgatggtctgtgacagccagtatttaatgaaaattattgtgaagaagtgcgccactgcctttgaaggcaagaggtccctgattgacgtcggtggcgggactggtggcgccgcacggtctattgccgaagcatttccagacatacaggaggtgtctgtattggatcttcctcatgtggttgcaggtttgcccaatgactcgagggtgaagtttgttggaggagacatgttccacaccatccctcccgctgatgttgtcttattgaaggcgatttttcatggttggaatgatgaggagtgcatcaagatattgaagaactgcaagaaggcaattccaagcaaggaagagggaggcaaggtgatgatattggacatggtggtcaattccgccccgggtgaccatatgattacagaagatcaatattttatggatttgatgatgataacctacgcaagaggattggagagagacgagaatgaatggaagaagctgtttaaagatgcaggtttcacatcgtacaagatcacccacgggcttggaacgagttcgcttatcgagctctacccttag

SEQ ID NO: 15 - DNA, 1536 bp, synthetic construct
  source 1..1536: mol_type = other DNA
atggagcaagctcaatgggtcgacccaactctgctccctgcatttgtgggcatcatcttcttcttccttggcatgttctttggaaggagttctttgggagctgggaagggtgcagcgcctagaagcaccagttctaccgagtggccagacggccctccaaagctgcccatcatcggcaacctgcaccagctcaacaaaggcggggagctggtccaccacaacctcgccaagctcgcccagtcctacgaccgcgccatgaccatctgggtcggcagctggggccccatgatcgtcgtcagcgacgccgaccttgcatgggaggtcctcgtcaccaagtcgccggacttcgccggccgggtgctctccaagctctcgcacttgttcaacgccaactacaacaccgtcgtcgcctacgacgccgggccgcaatggcagtcgctccggcgaggtctgcagcacgggccgctcggccccgcccatgtttctgcgcaggctcgtttccacgaagaagacatgaagctcctggtgagcgacatgatgagagcagcacagaaaggtggtagcaatggagtggttgaacctctggcctatgtccggcgagccactatccgatttctgtctcgtctatgctttggggaggccttcaacgacgaggcgttcgtggaggggatggacgaagcagtggaggagaccatcggagccactggccatgcacgcatcctcgacgccttctacttcactcgccacctccctatcatccgccgcagcttcatagataccgtcaacgccaagaagaagatcgagagccttgtccggccgttgctctcccggccggcgccaccggggtcttacctccacttcctcctttccaccgacgcgccggagaatatgatcatctttcgaatattcgaagtctacttgctgggcgtggacagcaccgcctccaccaccacatgggctctcgccttcctcgtctccaaccaacaggcgcaggagaagctccacaatgagctcgcccagtactgcgccagccagaacaatcagatcatcaaagcagacgacgtcggaaagctgtcgtacctgctcggggtagtgaaggagacgatgaggatgaagccgatagcgccgctggccgtcccccacaagacgctcaaggagacgatgctcgacggaaagagggtggcggcgggaacgacggtggtagtgaacctctatgccgtccactacaacccgaagctatggccggagccggagcagttccgcccggagaggttcgtggtcggcgccagcggcggcaatggtggggggtcttccgagtacatgctgcagtcgtacctgccctttggaggggggatgaggtcctgcgcagggatggaggtgggaaagttgcaggtggcgatggtcgtggccaaccttgtgatggcatttaaatggttgccggaggaggaggggaagatgccggacctggctgaagacatgaccttcgtgctcatgatgaagaagccattggctgccaaaatcgttccacgtgcatga

SEQ ID NO: 16 - DNA, 981 bp, synthetic construct
  source 1..981: mol_type = other DNA
atggagactgagaggaagtccaggatctgtgtcaccggggcaggaggctttgtagcctcttgggtcgtcaagcttttcctctccaaaggttatcttgtccatggcactgtcagagacctcggagaagagaagactgcccatttgaggaagttggagggtgcgtaccataatctgcagctgttcaaggctgacttgttggattatgagtccttgctcggggccattactggctgcgatggagttctccatgttgcaactcctgttccttcgagtaaaactgcttattccggaactgagttggtcaagactgctgtgaatggaactctgaatgtgctcagggcatgtacagaggcaaaagtgaaaaaggtcatctatgtttcatctactgccgctgttttggtgaatcctaatttacccaaggataaaatcccggacgaagattgttggacagacgaagagtactgcaggacaactccgttcttcctgaattggtattgcatcgccaaaacagcagccgaaaagaatgccttggaatatggagataaagaagggatcaacgttatatctatttgcccttcatacatctttggacctatgcttcaaccgacaattaattcaagcaacttggaattgttgaggctaatgaaaggagatgacgaaagcatagaaaacaaatttctgctgatggtggatgtgcgagatgttgctgaagcaattttactattatatgagaagcaagaaacatcagggagatacatttcttcgccgcatggtatgcgacaaagcaacttggttgagaagctggagagcctgcagccgggctacaattatcataagaactttgtggatattaaacctagttggacaatgatcagctcagaaaagctcaagaaacttggttggaaacctagaccacttgaggacactatttctgaaacagtgctgtgttttgaagagcatggtttgctggaaaatgaatag

(19) United States
(12) Patent Application Publication
(10) Pub. No.: US 2024/0294957 A1
(43) Pub. Date: Sep. 5, 2024

(54) ENZYMATIC SYNTHESIS OF KAVALACTONES AND FLAVOKAVAINS

(71) Applicant: Whitehead Institute for Biomedical Research, Cambridge, MA (US)

(72) Inventors: Tomás Pluskal, Boston, MA (US); Jing-Ke Weng, Belmont, MA (US)

(73) Assignee: Whitehead Institute for Biomedical Research, Cambridge, MA (US)

(21) Appl. No.: 18/350,985

(22) Filed: Jul. 12, 2023

Related U.S. Application Data
(63) Continuation of application No. 17/190,872, filed on Mar. 3, 2021, now Pat. No. 11,739,354.
Application No. 17/190,872 is a division of application No. 16/249,758, filed on Jan. 16, 2019, now Pat. No. 10,941,429.
(60) Provisional application No. 62/618,549, filed on Jan. 17, 2018.

(51) Int. Cl. C12P 17/06; C12P 7/26

(52) U.S. Cl. CPC C12P 17/06; C12P 7/26; C12Y 301/27005

(57) ABSTRACT

Disclosed are methods, compositions, proteins, nucleic acids, cells, vectors, compounds, reagents, and systems for the preparation of kavalactones, flavokavains, and kavalactone and flavokavain biosynthetic intermediates using enzymes expressed in heterologous host cells, such as microorganisms or plants, or using in vitro enzymatic reactions. This invention also provides for the expression of the enzymes by recombinant cell lines and vectors. Furthermore, the enzymes can be components of constructs such as fusion proteins. The kavalactones produced can be utilized to treat anxiety disorder, insomnia, and other psychological and neurological disorders. The flavokavains produced can be utilized to treat various cancers including colon, bladder, and breast cancers.

### RELATED APPLICATIONS

This application is a continuation of U.S. application Ser. No. 16/249,758, filed Jan. 16, 2019, which claims priority under 35 U.S.C. § 119(e) to U.S. Provisional Application No. 62/618,549, filed Jan. 17, 2018, each of which is incorporated by reference herein in its entirety.

### GOVERNMENT SUPPORT

This invention was made with government support under grant number 1709616 awarded by the National Science Foundation. The government has certain rights in the invention.

### BACKGROUND OF THE INVENTION

Kava or kava-kava (Piper methysticum) is a domesticated tropical shrub native to Oceania, where its roots are used to prepare a beverage for medicinal and ceremonial purposes.^{1,2} A substantial body of scientific evidence supports positive effects of kava in generalized anxiety disorder,³ insomnia,⁴ and other non-psychotic psychological and neurological disorders⁵. The main bioactive components of kava, called kavalactones (FIG. 1), are known to interact with the human nervous system via GABA_{A}, cannabinoid (CB₁), and other molecular receptors.⁶⁻⁸ Kava-kava is also known to contain several chalconoids called flavokavains (FIG. 2), which show promising anti-cancer activities against various cell lines including colon, bladder, and breast cancer.⁹ Therefore, there exists a need for ways to prepare kavalactones and flavokavains for research and medicinal purposes.

### SUMMARY OF THE INVENTION

Kavalactones, small hydrophobic polyketides, and flavokavains, chalconoids, are the bioactive ingredients found in the kava drink that is widely commercially available in the form of a dried kava-kava root powder. Despite a long tradition of kava consumption in its native Oceania, it occupies only a niche market in the Western society, likely due to the unpleasant taste of the kava drink. Since scientific literature has already established therapeutic and anxiolytic properties of kava, there is a potential to employ kavalactones as standalone supplements or as additives to various food products. Importantly, the effect on the human brain caused by kava consumption tends to be mild, and kava consumption is non-addictive.

Kavalactone structure is based on a styrylpyrone backbone decorated with various hydroxy and/or methoxy modifications. At least twenty different kavalactone structures are known, although six kavalactones are considered major ones, as together they constitute over 90% of the kavalactone content in the kava kava shrub. The biosynthetic pathway of kavalactones and flavokavains branches off the general phenylpropanoid pathway, utilizing coenzyme A (CoA) esters of cinnamic acids and malonyl-CoA as substrates for a type III polyketide synthase, which forms the structure backbone (FIG. 3). In the case of flavokavains, the chalcone backbone is produced in kava-kava by the chalcone synthase PmCHS. In the case of kavalactones, the corresponding 6-styryl-4-hydroxy-2-pyrone backbone is produced by one of two styrylpyrone synthases, PmSPS1 or PmSPS2. The backbones are further modified by decorating enzymes to produce individual kavalactones or flavokavains.

In the present disclosure, the elucidated native kavalactone biosynthetic pathway from Piper methysticum allows for the use of metabolic engineering to produce kavalactones and flavokavains through expression in heterologous hosts. Described herein are the methods, compounds, reagents, and systems constituting a bioengineering approach to sustainable production of kavalactones and flavokavains.

In one aspect, the present disclosure provides methods to produce kavalactones, flavokavains, and biosynthetic intermediates of kavalactones and flavokavains using enzymes at least 80% identical to naturally occurring enzymes of the kavalactone and flavokavain biosynthetic pathways.

In certain embodiments, methods are provided for the production of CoA esters of Formula (II) from carboxylic acids of Formula (I), or a salt thereof, and coenzyme A (CoA) using an enzyme that is at least 80% identical to Pm4CL1 (SEQ ID NO: 1). The structures of Formula (I) and Formula (II) are as follows:

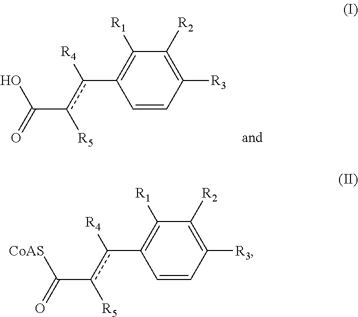

wherein: [symbol] is a single bond or a double bond; each of R₁, R₂, and R₃ independently is hydrogen, optionally substituted, cyclic or acyclic aliphatic, or ORₓ, wherein Rₓ is hydrogen or optionally substituted, cyclic or acyclic aliphatic; and each of R₄ and R₅ independently is hydrogen or optionally substituted, cyclic or acyclic aliphatic.

In certain embodiments, methods are provided for the production of compounds containing the kavalactone backbone (6-styryl-4-hydroxyl-2-pyrone) of Formula (III) from CoA esters of Formula (II), or a salt thereof, and malonyl-CoA using an enzyme that is at least 80% identical to PmSPS1 (SEQ ID NO: 2) or an enzyme that is at least 80% identical to PmSPS2 (SEQ ID NO: 3). The structures of Formula (II) and Formula (III) is as follows:

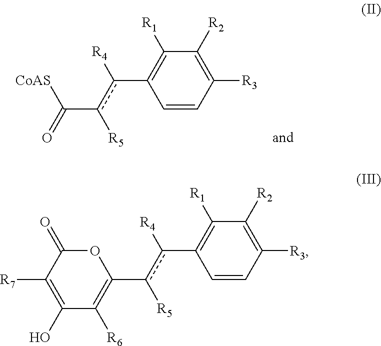

wherein: [symbol] is a single bond or a double bond; each of R₁, R₂, R₃, R₆, and R₇ independently is hydrogen, optionally substituted, cyclic or acyclic aliphatic, or ORₓ, or R₁ and R₂ are optionally combined to form a ring, or R₂ and R₃ are optionally combined to form a ring, wherein Rₓ is hydrogen or optionally substituted, cyclic or acyclic aliphatic; and each of R₄ and R₅ independently is hydrogen or optionally substituted, cyclic or acyclic aliphatic.

In certain embodiments, methods are provided for the production of methylated 6-styryl-4-hydroxyl-2-pyrone compounds of Formula (IV) from compounds containing the kavalactone backbone (6-styryl-4-hydroxyl-2-pyrone) of Formula (III), or a salt thereof, and S-adenosylmethionine using an enzyme that is at least 80% identical to PmOMT4 (SEQ ID NO: 5) or an enzyme that is at least 80% identical to PmOMT1 (SEQ ID NO: 6). The structures of Formula (III) and Formula (IV) are as follows:

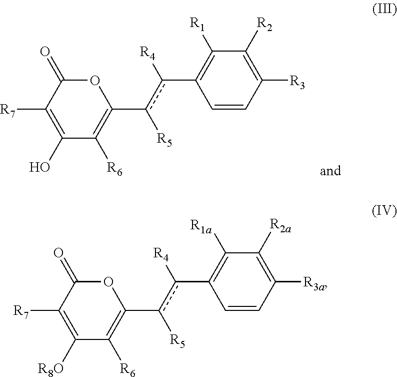

wherein: [symbol] is a single bond or a double bond; each of R₁, R₂, R₃, R₁ₐ, R₂ₐ, R₃ₐ, R₆, and R₇ independently is hydrogen, optionally substituted, cyclic or acyclic aliphatic, or ORₓ, or R₁ and R₂ are optionally combined to form a ring, or R₂ and R₃ are optionally combined to form a ring, or R₁ₐ and R₂ₐ are optionally combined to form a ring, or R₂ₐ and R₃ₐ are optionally combined to form a ring, wherein Rₓ is hydrogen or optionally substituted, cyclic or acyclic aliphatic; each of R₄ and R₅ independently is hydrogen or optionally substituted, cyclic or acyclic aliphatic; and R₈ is optionally substituted, cyclic or acyclic aliphatic.

In certain embodiments, methods are provided for the production of 6-styryl-4-hydroxyl-5,6-dihydro-2-pyrone compounds of Formula (V) from 6-styryl-4-hydroxyl-2-pyrone compounds of Formula (IV), or a salt thereof, and a reducing agent (i.e., NADPH or NADH) using an enzyme that is at least 80% identical to PmRDCT10 (SEQ ID NO: 8). The structures of Formula (IV) and Formula (V) are as follows:

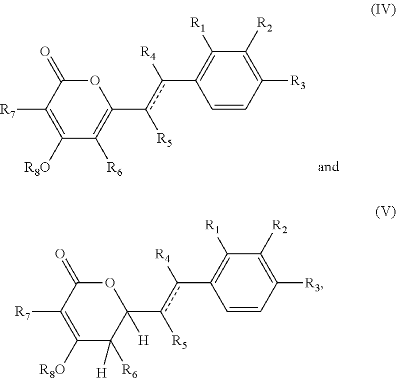

wherein: [symbol] is a single bond or a double bond; each of R₁, R₂, R₃, R₆, and R₇ independently is hydrogen, optionally substituted, cyclic or acyclic aliphatic, or ORₓ, or R₁ and R₂ are optionally combined to form a ring, or R₂ and R₃ are optionally combined to form a ring, wherein Rₓ is hydrogen or optionally substituted, cyclic or acyclic aliphatic; each of R₄ and R₅ independently is hydrogen or optionally substituted, cyclic or acyclic aliphatic; and R₈ is optionally substituted, cyclic or acyclic aliphatic.

In certain embodiments, methods are provided for the production of 6-styryl-4-hydroxyl-2-pyrone compounds containing a methylenedioxy bridge of Formula (VI) from 6-styryl-4-hydroxyl-2-pyrone compounds of Formula (IV), or salt thereof, and a reducing agent (i.e., NADPH or NADH) using an enzyme that is at least 80% identical to PmMDB1 (SEQ ID NO: 7). The structures of Formula (IV) and Formula (VI) are as follows:

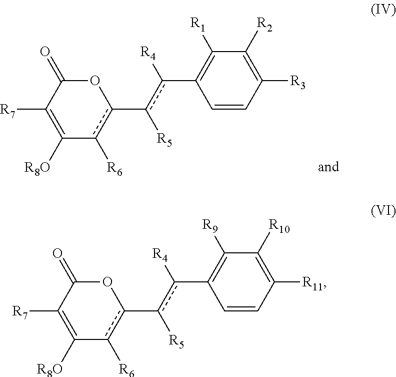

wherein: [symbol] is a single bond or a double bond; each of R₁, R₂, R₃, R₆, R₇, R₉, R₁₀, and R₁₁ independently is hydrogen, optionally substituted, cyclic or acyclic aliphatic, or ORₓ, or R₁ and R₂ are combined to form a ring, or R₂ and R₃ are combined to form a ring, R₉ and R₁₀ are combined to form a ring, or R₁₀ and R₁₁ are combined to form a ring, wherein Rₓ is hydrogen or optionally substituted, cyclic or acyclic aliphatic; each of R₄ and R₅ independently is hydrogen or optionally substituted, cyclic or acyclic aliphatic; and R₈ is optionally substituted, cyclic or acyclic aliphatic; provided that at least one of R₁, R₂, or R₃ is —OH, at least one of R₁, R₂, or R₃ is —OMe, and R₉ and R₁₀ or R₁₀ and R₁₁ are combined to form a methylenedioxy cyclic moiety —OCH₂O—.

In certain embodiments, methods are provided for the production of compounds containing the chalcone backbone of Formula (VII) from CoA esters of Formula (II), or a salt thereof, and malonyl-CoA using an enzyme that is at least 80% identical to PmCHS (SEQ ID NO: 4). The structures of Formula (II) and Formula (VII) are as follows:

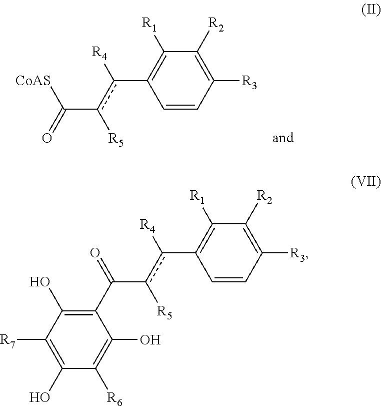

wherein: [symbol] is a single bond or a double bond; and each of R₁, R₂, R₃ R₆, and R₇ independently is hydrogen, optionally substituted, cyclic or acyclic aliphatic, or ORₓ, wherein Rₓ is hydrogen or optionally substituted, cyclic or acyclic aliphatic; and each of R₄ and R₅ independently is hydrogen or optionally substituted, cyclic or acyclic aliphatic.

In certain embodiments, methods are provided for the production of methylated chalcone compounds of Formula (VIII) from compounds containing the chalcone backbone of Formula (VII), or a salt thereof, and S-adenosylmethionine using an enzyme that is at least 80% identical to PmOMT4 (SEQ ID NO: 5) or an enzyme that is at least 80% identical to PmOMT1 (SEQ ID NO: 6). The structures of Formula (VII) and Formula (VIII) are as follows:

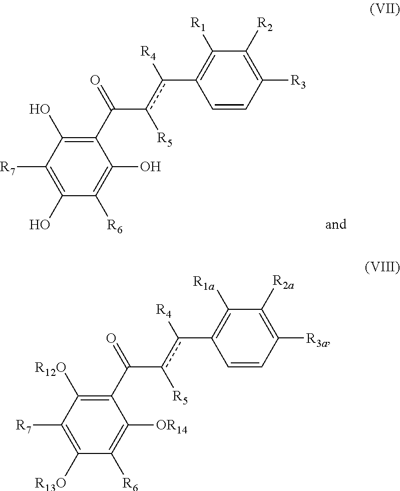

wherein: [symbol] is a single bond or a double bond; and each of R₁, R₂, R₃, R₁ₐ, R₂ₐ, R₃ₐ, R₆, and R₇ independently is hydrogen, optionally substituted, cyclic or acyclic aliphatic, or ORₓ, wherein Rₓ is hydrogen or optionally substituted, cyclic or acyclic aliphatic; each of R₄ and R₅ independently is hydrogen or optionally substituted, cyclic or acyclic aliphatic; each of R₁₂ and R₁₃ independently is optionally substituted, cyclic or acyclic aliphatic; and R₁₄ is hydrogen.

In another aspect, the present disclosure provides recombinant nucleic acids encoding for the enzymes described herein (e.g., 4-coumarate-CoA ligase Pm4CL1 or an enzyme that is at least 80% identical to SEQ ID NO: 1, styrylpyrone synthase PmSPS1 or an enzyme that is at least 80% identical to SEQ ID NO: 2, styrylpyrone synthase PmSPS2 or an enzyme that is at least 80% identical to SEQ ID NO: 3, chalcone synthase PmCHS or an enzyme that is at least 80% identical to SEQ ID NO: 4, methyltransferase PmOMT4 or an enzyme that is at least 80% identical to SEQ ID NO: 5, methyltransferase PmOMT1 or an enzyme that is at least 80% identical to SEQ ID NO: 6, cytochrome P450 enzyme PmMDB1 or an enzyme that is at least 80% identical to SEQ ID NO: 7, and NADPH-dependent reductase PmRDCT10 or an enzyme that is at least 80% identical to SEQ ID NO: 8). In certain embodiments, the recombinant nucleic acids are complementary DNA (cDNA) molecules. The cDNA molecules may be contained in vectors. These vectors may be transferred into host cells or organisms including, but not limited to, bacteria, yeast, and plants. In certain embodiments, the host is a bacterium and is a wildtype, mutant, recombinant, or genetically engineered form of Escherichia coli. In certain embodiments, the host is a yeast and is a wildtype, mutant, recombinant, or genetically engineered form of Saccharomyces cerevisiae. In certain embodiments, the host is a plant is a wildtype, mutant, recombinant, or genetically engineered form of Nicotiana benthamiana.

The details of certain embodiments of the invention are set forth in the Detailed Description of Certain Embodiments, as described below. Other features, objects, and advantages of the invention will be apparent from the Definitions, Examples, Figures, and Claims.

### Definitions

Definitions of specific functional groups and chemical terms are described in more detail below. The chemical elements are identified in accordance with the Periodic Table of the Elements, CAS version, Handbook of Chemistry and Physics, 75^{th} Ed., inside cover, and specific functional groups are generally defined as described therein. Additionally, general principles of organic chemistry, as well as specific functional moieties and reactivity, are described in Organic Chemistry, Thomas Sorrell, University Science Books, Sausalito, 1999; Smith and March, March's Advanced Organic Chemistry, 5^{th} Edition, John Wiley & Sons, Inc., New York, 2001; Larock, Comprehensive Organic Transformations, VCH Publishers, Inc., New York, 1989; and Carruthers, Some Modern Methods of Organic Synthesis, 3^{rd} Edition, Cambridge University Press, Cambridge, 1987.

Compounds described herein can comprise one or more asymmetric centers, and thus can exist in various stereoisomeric forms, e.g., enantiomers and/or diastereomers. For example, the compounds described herein can be in the form of an individual enantiomer, diastereomer or geometric isomer, or can be in the form of a mixture of stereoisomers, including racemic mixtures and mixtures enriched in one or more stereoisomer. Isomers can be isolated from mixtures by methods known to those skilled in the art, including chiral high pressure liquid chromatography (HPLC) and the formation and crystallization of chiral salts; or preferred isomers can be prepared by asymmetric syntheses. See, for example, Jacques et al., Enantiomers, Racemates and Resolutions (Wiley Interscience, New York, 1981); Wilen et al., Tetrahedron 33:2725 (1977); Eliel, E. L. Stereochemistry of Carbon Compounds (McGraw-Hill, N Y, 1962); and Wilen, S. H., Tables of Resolving Agents and Optical Resolutions p. 268 (E. L. Eliel, Ed., Univ. of Notre Dame Press, Notre Dame, IN 1972). The invention additionally encompasses compounds as individual isomers substantially free of other isomers, and alternatively, as mixtures of various isomers.

In a formula, [symbol] is a single bond where the stereochemistry of the moieties immediately attached thereto is not specified, [symbol] is absent or a single bond, and [symbol] or [symbol] is a single or double bond.

Unless otherwise stated, structures depicted herein are also meant to include compounds that differ only in the presence of one or more isotopically enriched atoms. For example, compounds having the present structures except for the replacement of hydrogen by deuterium or tritium, replacement of ¹⁹F with ¹⁸F, or the replacement of a carbon by a ¹³C- or ¹⁴C-enriched carbon are within the scope of the disclosure. Such compounds are useful, for example, as analytical tools or probes in biological assays.

When a range of values is listed, it is intended to encompass each value and subrange within the range. For example, “C₁₋₆ alkyl” is intended to encompass, C₁, C₂, C₃, C₄, C₅, C₆, C₁₋₆, C₁₋₅, C₁₋₄, C₁₋₃, C₁₋₂, C₂₋₆, C₂₋₅, C₂₋₄, C₂₋₃, C₃₋₆, C₃₋₅, C₃₋₄, C₄₋₆, C₄₋₅, and C₅₋₆ alkyl.

The term “aliphatic” refers to alkyl, alkenyl, alkynyl, and carbocyclic groups. Likewise, the term “heteroaliphatic” refers to heteroalkyl, heteroalkenyl, heteroalkynyl, and heterocyclic groups.

The term “alkyl” refers to a radical of a straight-chain or branched saturated hydrocarbon group having from 1 to 10 carbon atoms (“C₁₋₁₀ alkyl”). In some embodiments, an alkyl group has 1 to 9 carbon atoms (“C₁₋₉ alkyl”). In some embodiments, an alkyl group has 1 to 8 carbon atoms (“C₁₋₈ alkyl”). In some embodiments, an alkyl group has 1 to 7 carbon atoms (“C₁₋₇ alkyl”). In some embodiments, an alkyl group has 1 to 6 carbon atoms (“C₁₋₆ alkyl”). In some embodiments, an alkyl group has 1 to 5 carbon atoms (“C₁₋₅ alkyl”). In some embodiments, an alkyl group has 1 to 4 carbon atoms (“C₁₋₄ alkyl”). In some embodiments, an alkyl group has 1 to 3 carbon atoms (“C₁₋₃ alkyl”). In some embodiments, an alkyl group has 1 to 2 carbon atoms (“C₁₋₂ alkyl”). In some embodiments, an alkyl group has 1 carbon atom (“C₁ alkyl”). In some embodiments, an alkyl group has 2 to 6 carbon atoms (“C₂₋₆ alkyl”). Examples of C₁₋₆ alkyl groups include methyl (C₁), ethyl (C₂), propyl (C₃) (e.g., n-propyl, isopropyl), butyl (C₄) (e.g., n-butyl, tert-butyl, sec-butyl, iso-butyl), pentyl (C₅) (e.g., n-pentyl, 3-pentanyl, amyl, neopentyl, 3-methyl-2-butanyl, tertiary amyl), and hexyl (C₆) (e.g., n-hexyl). Additional examples of alkyl groups include n-heptyl (C₇), n-octyl (C₈), and the like. Unless otherwise specified, each instance of an alkyl group is independently unsubstituted (an “unsubstituted alkyl”) or substituted (a “substituted alkyl”) with one or more substituents (e.g., halogen, such as F). In certain embodiments, the alkyl group is an unsubstituted C₁₋₁₀ alkyl (such as unsubstituted C₁₋₆ alkyl, e.g., —CH₃ (Me), unsubstituted ethyl (Et), unsubstituted propyl (Pr, e.g., unsubstituted n-propyl (n-Pr), unsubstituted isopropyl (i-Pr)), unsubstituted butyl (Bu, e.g., unsubstituted n-butyl (n-Bu), unsubstituted tert-butyl (tert-Bu or t-Bu), unsubstituted sec-butyl (sec-Bu or s-Bu), unsubstituted isobutyl (i-Bu)). In certain embodiments, the alkyl group is a substituted C₁₋₁₀ alkyl (such as substituted C₁₋₆ alkyl, e.g., —CH₂F, —CHF₂, —CF₃ or benzyl (Bn)).

The term “haloalkyl” is a substituted alkyl group, wherein one or more of the hydrogen atoms are independently replaced by a halogen, e.g., fluoro, bromo, chloro, or iodo. “Perhaloalkyl” is a subset of haloalkyl, and refers to an alkyl group wherein all of the hydrogen atoms are independently replaced by a halogen, e.g., fluoro, bromo, chloro, or iodo. In some embodiments, the haloalkyl moiety has 1 to 8 carbon atoms (“C₁₋₈ haloalkyl”). In some embodiments, the haloalkyl moiety has 1 to 6 carbon atoms (“C₁₋₆ haloalkyl”). In some embodiments, the haloalkyl moiety has 1 to 4 carbon atoms (“C₁₋₄ haloalkyl”). In some embodiments, the haloalkyl moiety has 1 to 3 carbon atoms (“C₁₋₃ haloalkyl”). In some embodiments, the haloalkyl moiety has 1 to 2 carbon atoms (“C₁₋₂ haloalkyl”). In some embodiments, all of the haloalkyl hydrogen atoms are replaced with fluoro to provide a perfluoroalkyl group. In some embodiments, all of the haloalkyl hydrogen atoms are replaced with chloro to provide a “perchloroalkyl” group. Examples of haloalkyl groups include —CF₃, —CF₂CF₃, —CF₂CF₂CF₃, —CCl₃, —CFCl₂, —CF₂C₁, and the like.

The term “heteroalkyl” refers to an alkyl group, which further includes at least one heteroatom (e.g., 1, 2, 3, or 4 heteroatoms) selected from oxygen, nitrogen, or sulfur within (i.e., inserted between adjacent carbon atoms of) and/or placed at one or more terminal position(s) of the parent chain. In certain embodiments, a heteroalkyl group refers to a saturated group having from 1 to 10 carbon atoms and 1 or more heteroatoms within the parent chain (“heteroC₁₋₁₀ alkyl”). In some embodiments, a heteroalkyl group is a saturated group having 1 to 9 carbon atoms and 1 or more heteroatoms within the parent chain (“heteroC₁₋₉ alkyl”). In some embodiments, a heteroalkyl group is a saturated group having 1 to 8 carbon atoms and 1 or more heteroatoms within the parent chain (“heteroC₁₋₈ alkyl”). In some embodiments, a heteroalkyl group is a saturated group having 1 to 7 carbon atoms and 1 or more heteroatoms within the parent chain (“heteroC₁₋₇ alkyl”). In some embodiments, a heteroalkyl group is a saturated group having 1 to 6 carbon atoms and 1 or more heteroatoms within the parent chain (“heteroC₁₋₆ alkyl”). In some embodiments, a heteroalkyl group is a saturated group having 1 to 5 carbon atoms and 1 or 2 heteroatoms within the parent chain (“heteroC₁₋₅ alkyl”). In some embodiments, a heteroalkyl group is a saturated group having 1 to 4 carbon atoms and 1 or 2 heteroatoms within the parent chain (“heteroC₁₋₄ alkyl”). In some embodiments, a heteroalkyl group is a saturated group having 1 to 3 carbon atoms and 1 heteroatom within the parent chain (“heteroC₁₋₃ alkyl”). In some embodiments, a heteroalkyl group is a saturated group having 1 to 2 carbon atoms and 1 heteroatom within the parent chain (“heteroC₁₋₂ alkyl”). In some embodiments, a heteroalkyl group is a saturated group having 1 carbon atom and 1 heteroatom (“heteroC₁ alkyl”). In some embodiments, a heteroalkyl group is a saturated group having 2 to 6 carbon atoms and 1 or 2 heteroatoms within the parent chain (“heteroC₂₋₆ alkyl”). Unless otherwise specified, each instance of a heteroalkyl group is independently unsubstituted (an “unsubstituted heteroalkyl”) or substituted (a “substituted heteroalkyl”) with one or more substituents. In certain embodiments, the heteroalkyl group is an unsubstituted heteroC₁₋₁₀ alkyl. In certain embodiments, the heteroalkyl group is a substituted heteroC₁₋₁₀ alkyl.

The term “alkenyl” refers to a radical of a straight-chain or branched hydrocarbon group having from 2 to 10 carbon atoms and one or more carbon-carbon double bonds (e.g., 1, 2, 3, or 4 double bonds). In some embodiments, an alkenyl group has 2 to 9 carbon atoms (“C₂₋₉ alkenyl”). In some embodiments, an alkenyl group has 2 to 8 carbon atoms (“C₂₋₈ alkenyl”). In some embodiments, an alkenyl group has 2 to 7 carbon atoms (“C₂₋₇ alkenyl”). In some embodiments, an alkenyl group has 2 to 6 carbon atoms (“C₂₋₆ alkenyl”). In some embodiments, an alkenyl group has 2 to 5 carbon atoms (“C₂₋₅ alkenyl”). In some embodiments, an alkenyl group has 2 to 4 carbon atoms (“C₂₋₄ alkenyl”). In some embodiments, an alkenyl group has 2 to 3 carbon atoms (“C₂₋₃ alkenyl”). In some embodiments, an alkenyl group has 2 carbon atoms (“C₂ alkenyl”). The one or more carbon-carbon double bonds can be internal (such as in 2-butenyl) or terminal (such as in 1-butenyl). Examples of C₂₋₄ alkenyl groups include ethenyl (C₂), 1-propenyl (C₃), 2-propenyl (C₃), 1-butenyl (C₄), 2-butenyl (C₄), butadienyl (C₄), and the like. Examples of C₂₋₆ alkenyl groups include the aforementioned C₂₋₄ alkenyl groups as well as pentenyl (C₅), pentadienyl (C₅), hexenyl (C₆), and the like. Additional examples of alkenyl include heptenyl (C₇), octenyl (C₈), octatrienyl (C₈), and the like. Unless otherwise specified, each instance of an alkenyl group is independently unsubstituted (an “unsubstituted alkenyl”) or substituted (a “substituted alkenyl”) with one or more substituents. In certain embodiments, the alkenyl group is an unsubstituted C₂₋₁₀ alkenyl. In certain embodiments, the alkenyl group is a substituted C₂₋₁₀ alkenyl. In an alkenyl group, a C═C double bond for which the stereochemistry is not specified (e.g., —CH═CHCH₃,

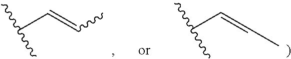

may be in the (E)- or (Z)-configuration.

The term “heteroalkenyl” refers to an alkenyl group, which further includes at least one heteroatom (e.g., 1, 2, 3, or 4 heteroatoms) selected from oxygen, nitrogen, or sulfur within (i.e., inserted between adjacent carbon atoms of) and/or placed at one or more terminal position(s) of the parent chain. In certain embodiments, a heteroalkenyl group refers to a group having from 2 to 10 carbon atoms, at least one double bond, and 1 or more heteroatoms within the parent chain (“heteroC₂₋₁₀alkenyl”). In some embodiments, a heteroalkenyl group has 2 to 9 carbon atoms at least one double bond, and 1 or more heteroatoms within the parent chain (“heteroC₂₋₉ alkenyl”). In some embodiments, a heteroalkenyl group has 2 to 8 carbon atoms, at least one double bond, and 1 or more heteroatoms within the parent chain (“heteroC₂₋₈ alkenyl”). In some embodiments, a heteroalkenyl group has 2 to 7 carbon atoms, at least one double bond, and 1 or more heteroatoms within the parent chain (“heteroC₂₋₇ alkenyl”). In some embodiments, a heteroalkenyl group has 2 to 6 carbon atoms, at least one double bond, and 1 or more heteroatoms within the parent chain (“heteroC₂₋₆ alkenyl”). In some embodiments, a heteroalkenyl group has 2 to 5 carbon atoms, at least one double bond, and 1 or 2 heteroatoms within the parent chain (“heteroC₂₋₅ alkenyl”). In some embodiments, a heteroalkenyl group has 2 to 4 carbon atoms, at least one double bond, and 1 or 2 heteroatoms within the parent chain (“heteroC₂₋₄ alkenyl”). In some embodiments, a heteroalkenyl group has 2 to 3 carbon atoms, at least one double bond, and 1 heteroatom within the parent chain (“heteroC₂₋₃ alkenyl”). In some embodiments, a heteroalkenyl group has 2 to 6 carbon atoms, at least one double bond, and 1 or 2 heteroatoms within the parent chain (“heteroC₂₋₆ alkenyl”). Unless otherwise specified, each instance of a heteroalkenyl group is independently unsubstituted (an “unsubstituted heteroalkenyl”) or substituted (a “substituted heteroalkenyl”) with one or more substituents. In certain embodiments, the heteroalkenyl group is an unsubstituted heteroC₂₋₁₀alkenyl. In certain embodiments, the heteroalkenyl group is a substituted heteroC₂₋₁₀alkenyl.

The term “alkynyl” refers to a radical of a straight-chain or branched hydrocarbon group having from 2 to 10 carbon atoms and one or more carbon-carbon triple bonds (e.g., 1, 2, 3, or 4 triple bonds) (“C₂₋₁₀ alkynyl”). In some embodiments, an alkynyl group has 2 to 9 carbon atoms (“C₂₋₉ alkynyl”). In some embodiments, an alkynyl group has 2 to 8 carbon atoms (“C₂₋₈ alkynyl”). In some embodiments, an alkynyl group has 2 to 7 carbon atoms (“C₂₋₇ alkynyl”). In some embodiments, an alkynyl group has 2 to 6 carbon atoms (“C₂₋₆ alkynyl”). In some embodiments, an alkynyl group has 2 to 5 carbon atoms (“C₂₋₅ alkynyl”). In some embodiments, an alkynyl group has 2 to 4 carbon atoms (“C₂₋₄ alkynyl”). In some embodiments, an alkynyl group has 2 to 3 carbon atoms (“C₂₋₃ alkynyl”). In some embodiments, an alkynyl group has 2 carbon atoms (“C₂ alkynyl”). The one or more carbon-carbon triple bonds can be internal (such as in 2-butynyl) or terminal (such as in 1-butynyl). Examples of C₂₋₄ alkynyl groups include, without limitation, ethynyl (C₂), 1-propynyl (C₃), 2-propynyl (C₃), 1-butynyl (C₄), 2-butynyl (C₄), and the like. Examples of C₂₋₆ alkenyl groups include the aforementioned C₂₋₄ alkynyl groups as well as pentynyl (C₅), hexynyl (C₆), and the like. Additional examples of alkynyl include heptynyl (C₇), octynyl (C₈), and the like. Unless otherwise specified, each instance of an alkynyl group is independently unsubstituted (an “unsubstituted alkynyl”) or substituted (a “substituted alkynyl”) with one or more substituents. In certain embodiments, the alkynyl group is an unsubstituted C₂₋₁₀ alkynyl. In certain embodiments, the alkynyl group is a substituted C₂₋₁₀ alkynyl.

The term “heteroalkynyl” refers to an alkynyl group, which further includes at least one heteroatom (e.g., 1, 2, 3, or 4 heteroatoms) selected from oxygen, nitrogen, or sulfur within (i.e., inserted between adjacent carbon atoms of) and/or placed at one or more terminal position(s) of the parent chain. In certain embodiments, a heteroalkynyl group refers to a group having from 2 to 10 carbon atoms, at least one triple bond, and 1 or more heteroatoms within the parent chain (“heteroC₂₋₁₀alkynyl”). In some embodiments, a heteroalkynyl group has 2 to 9 carbon atoms, at least one triple bond, and 1 or more heteroatoms within the parent chain (“heteroC₂₋₉ alkynyl”). In some embodiments, a heteroalkynyl group has 2 to 8 carbon atoms, at least one triple bond, and 1 or more heteroatoms within the parent chain (“heteroC₂₋₈ alkynyl”). In some embodiments, a heteroalkynyl group has 2 to 7 carbon atoms, at least one triple bond, and 1 or more heteroatoms within the parent chain (“heteroC₂₋₇ alkynyl”). In some embodiments, a heteroalkynyl group has 2 to 6 carbon atoms, at least one triple bond, and 1 or more heteroatoms within the parent chain (“heteroC₂₋₆ alkynyl”). In some embodiments, a heteroalkynyl group has 2 to 5 carbon atoms, at least one triple bond, and 1 or 2 heteroatoms within the parent chain (“heteroC₂₋₅ alkynyl”). In some embodiments, a heteroalkynyl group has 2 to 4 carbon atoms, at least one triple bond, and 1 or 2 heteroatoms within the parent chain (“heteroC₂₋₄ alkynyl”). In some embodiments, a heteroalkynyl group has 2 to 3 carbon atoms, at least one triple bond, and 1 heteroatom within the parent chain (“heteroC₂₋₃ alkynyl”). In some embodiments, a heteroalkynyl group has 2 to 6 carbon atoms, at least one triple bond, and 1 or 2 heteroatoms within the parent chain (“heteroC₂₋₆ alkynyl”). Unless otherwise specified, each instance of a heteroalkynyl group is independently unsubstituted (an “unsubstituted heteroalkynyl”) or substituted (a “substituted heteroalkynyl”) with one or more substituents. In certain embodiments, the heteroalkynyl group is an unsubstituted heteroC₂₋₁₀ alkynyl. In certain embodiments, the heteroalkynyl group is a substituted heteroC₂₋₁₀ alkynyl.

The term “carbocyclyl” or “carbocyclic” refers to a radical of a non-aromatic cyclic hydrocarbon group having from 3 to 14 ring carbon atoms (“C₃₋₁₄ carbocyclyl”) and zero heteroatoms in the non-aromatic ring system. In some embodiments, a carbocyclyl group has 3 to 10 ring carbon atoms (“C₃₋₁₀ carbocyclyl”). In some embodiments, a carbocyclyl group has 3 to 8 ring carbon atoms (“C₃₋₈ carbocyclyl”). In some embodiments, a carbocyclyl group has 3 to 7 ring carbon atoms (“C₃₋₇ carbocyclyl”). In some embodiments, a carbocyclyl group has 3 to 6 ring carbon atoms (“C₃₋₆ carbocyclyl”). In some embodiments, a carbocyclyl group has 4 to 6 ring carbon atoms (“C₄₋₆ carbocyclyl”). In some embodiments, a carbocyclyl group has 5 to 6 ring carbon atoms (“C₅₋₆ carbocyclyl”). In some embodiments, a carbocyclyl group has 5 to 10 ring carbon atoms (“C₅₋₁₀ carbocyclyl”). Exemplary C₃₋₆ carbocyclyl groups include, without limitation, cyclopropyl (C₃), cyclopropenyl (C₃), cyclobutyl (C₄), cyclobutenyl (C₄), cyclopentyl (C₅), cyclopentenyl (C₅), cyclohexyl (C₆), cyclohexenyl (C₆), cyclohexadienyl (C₆), and the like. Exemplary C₃₋₈ carbocyclyl groups include, without limitation, the aforementioned C₃₋₆ carbocyclyl groups as well as cycloheptyl (C₇), cycloheptenyl (C₇), cycloheptadienyl (C₇), cycloheptatrienyl (C₇), cyclooctyl (C₈), cyclooctenyl (C₈), bicyclo[2.2.1]heptanyl (C₇), bicyclo[2.2.2]octanyl (C₈), and the like. Exemplary C₃₋₁₀ carbocyclyl groups include, without limitation, the aforementioned C₃₋₈ carbocyclyl groups as well as cyclononyl (C₉), cyclononenyl (C₉), cyclodecyl (C₁₀), cyclodecenyl (C₁₀), octahydro-1H-indenyl (C₉), decahydronaphthalenyl (C₁₀), spiro[4.5]decanyl (C₁₀), and the like. As the foregoing examples illustrate, in certain embodiments, the carbocyclyl group is either monocyclic (“monocyclic carbocyclyl”) or polycyclic (e.g., containing a fused, bridged or spiro ring system such as a bicyclic system (“bicyclic carbocyclyl”) or tricyclic system (“tricyclic carbocyclyl”)) and can be saturated or can contain one or more carbon-carbon double or triple bonds. “Carbocyclyl” also includes ring systems wherein the carbocyclyl ring, as defined above, is fused with one or more aryl or heteroaryl groups wherein the point of attachment is on the carbocyclyl ring, and in such instances, the number of carbons continue to designate the number of carbons in the carbocyclic ring system. Unless otherwise specified, each instance of a carbocyclyl group is independently unsubstituted (an “unsubstituted carbocyclyl”) or substituted (a “substituted carbocyclyl”) with one or more substituents. In certain embodiments, the carbocyclyl group is an unsubstituted C₃₋₁₄ carbocyclyl. In certain embodiments, the carbocyclyl group is a substituted C₃₋₁₄ carbocyclyl.

In some embodiments, “carbocyclyl” is a monocyclic, saturated carbocyclyl group having from 3 to 14 ring carbon atoms (“C₃₋₁₄ cycloalkyl”). In some embodiments, a cycloalkyl group has 3 to 10 ring carbon atoms (“C₃₋₁₀ cycloalkyl”). In some embodiments, a cycloalkyl group has 3 to 8 ring carbon atoms (“C₃₋₈ cycloalkyl”). In some embodiments, a cycloalkyl group has 3 to 6 ring carbon atoms (“C₃₋₆ cycloalkyl”). In some embodiments, a cycloalkyl group has 4 to 6 ring carbon atoms (“C₄₋₆ cycloalkyl”). In some embodiments, a cycloalkyl group has 5 to 6 ring carbon atoms (“C₅₋₆ cycloalkyl”). In some embodiments, a cycloalkyl group has 5 to 10 ring carbon atoms (“C₅₋₁₀ cycloalkyl”). Examples of C₅₋₆ cycloalkyl groups include cyclopentyl (C₅) and cyclohexyl (C₅). Examples of C₃₋₆ cycloalkyl groups include the aforementioned C₅₋₆ cycloalkyl groups as well as cyclopropyl (C₃) and cyclobutyl (C₄). Examples of C₃₋₈ cycloalkyl groups include the aforementioned C₃₋₆ cycloalkyl groups as well as cycloheptyl (C₇) and cyclooctyl (C₈). Unless otherwise specified, each instance of a cycloalkyl group is independently unsubstituted (an “unsubstituted cycloalkyl”) or substituted (a “substituted cycloalkyl”) with one or more substituents. In certain embodiments, the cycloalkyl group is an unsubstituted C₃₋₁₄ cycloalkyl. In certain embodiments, the cycloalkyl group is a substituted C₃₋₁₄ cycloalkyl. In certain embodiments, the carbocyclyl includes 0, 1, or 2 C═C double bonds in the carbocyclic ring system, as valency permits.

The term “heterocyclyl” or “heterocyclic” refers to a radical of a 3- to 14-membered non-aromatic ring system having ring carbon atoms and 1 to 4 ring heteroatoms, wherein each heteroatom is independently selected from nitrogen, oxygen, and sulfur (“3-14 membered heterocyclyl”). In heterocyclyl groups that contain one or more nitrogen atoms, the point of attachment can be a carbon or nitrogen atom, as valency permits. A heterocyclyl group can either be monocyclic (“monocyclic heterocyclyl”) or polycyclic (e.g., a fused, bridged or spiro ring system such as a bicyclic system (“bicyclic heterocyclyl”) or tricyclic system (“tricyclic heterocyclyl”)), and can be saturated or can contain one or more carbon-carbon double or triple bonds. Heterocyclyl polycyclic ring systems can include one or more heteroatoms in one or both rings. “Heterocyclyl” also includes ring systems wherein the heterocyclyl ring, as defined above, is fused with one or more carbocyclyl groups wherein the point of attachment is either on the carbocyclyl or heterocyclyl ring, or ring systems wherein the heterocyclyl ring, as defined above, is fused with one or more aryl or heteroaryl groups, wherein the point of attachment is on the heterocyclyl ring, and in such instances, the number of ring members continue to designate the number of ring members in the heterocyclyl ring system. Unless otherwise specified, each instance of heterocyclyl is independently unsubstituted (an “unsubstituted heterocyclyl”) or substituted (a “substituted heterocyclyl”) with one or more substituents. In certain embodiments, the heterocyclyl group is an unsubstituted 3-14 membered heterocyclyl. In certain embodiments, the heterocyclyl group is a substituted 3-14 membered heterocyclyl. In certain embodiments, the heterocyclyl is substituted or unsubstituted, 3- to 7-membered, monocyclic heterocyclyl, wherein 1, 2, or 3 atoms in the heterocyclic ring system are independently oxygen, nitrogen, or sulfur, as valency permits.

In some embodiments, a heterocyclyl group is a 5-10 membered non-aromatic ring system having ring carbon atoms and 1-4 ring heteroatoms, wherein each heteroatom is independently selected from nitrogen, oxygen, and sulfur (“5-10 membered heterocyclyl”). In some embodiments, a heterocyclyl group is a 5-8 membered non-aromatic ring system having ring carbon atoms and 1-4 ring heteroatoms, wherein each heteroatom is independently selected from nitrogen, oxygen, and sulfur (“5-8 membered heterocyclyl”). In some embodiments, a heterocyclyl group is a 5-6 membered non-aromatic ring system having ring carbon atoms and 1-4 ring heteroatoms, wherein each heteroatom is independently selected from nitrogen, oxygen, and sulfur (“5-6 membered heterocyclyl”). In some embodiments, the 5-6 membered heterocyclyl has 1-3 ring heteroatoms selected from nitrogen, oxygen, and sulfur. In some embodiments, the 5-6 membered heterocyclyl has 1-2 ring heteroatoms selected from nitrogen, oxygen, and sulfur. In some embodiments, the 5-6 membered heterocyclyl has 1 ring heteroatom selected from nitrogen, oxygen, and sulfur.

Exemplary 3-membered heterocyclyl groups containing 1 heteroatom include, without limitation, azirdinyl, oxiranyl, and thiiranyl. Exemplary 4-membered heterocyclyl groups containing 1 heteroatom include, without limitation, azetidinyl, oxetanyl, and thietanyl. Exemplary 5-membered heterocyclyl groups containing 1 heteroatom include, without limitation, tetrahydrofuranyl, dihydrofuranyl, tetrahydrothiophenyl, dihydrothiophenyl, pyrrolidinyl, dihydropyrrolyl, and pyrrolyl-2,5-dione. Exemplary 5-membered heterocyclyl groups containing 2 heteroatoms include, without limitation, dioxolanyl, oxathiolanyl and dithiolanyl. Exemplary 5-membered heterocyclyl groups containing 3 heteroatoms include, without limitation, triazolinyl, oxadiazolinyl, and thiadiazolinyl. Exemplary 6-membered heterocyclyl groups containing 1 heteroatom include, without limitation, piperidinyl, tetrahydropyranyl, dihydropyridinyl, and thianyl. Exemplary 6-membered heterocyclyl groups containing 2 heteroatoms include, without limitation, piperazinyl, morpholinyl, dithianyl, and dioxanyl. Exemplary 6-membered heterocyclyl groups containing 3 heteroatoms include, without limitation, triazinyl. Exemplary 7-membered heterocyclyl groups containing 1 heteroatom include, without limitation, azepanyl, oxepanyl and thiepanyl. Exemplary 8-membered heterocyclyl groups containing 1 heteroatom include, without limitation, azocanyl, oxecanyl and thiocanyl. Exemplary bicyclic heterocyclyl groups include, without limitation, indolinyl, isoindolinyl, dihydrobenzofuranyl, dihydrobenzothienyl, tetrahydrobenzothienyl, tetrahydrobenzofuranyl, tetrahydroindolyl, tetrahydroquinolinyl, tetrahydroisoquinolinyl, decahydroquinolinyl, decahydroisoquinolinyl, octahydrochromenyl, octahydroisochromenyl, decahydronaphthyridinyl, decahydro-1,8-naphthyridinyl, octahydropyrrolo[3,2-b]pyrrole, indolinyl, phthalimidyl, naphthalimidyl, chromanyl, chromenyl, 1H-benzo[e][1,4]diazepinyl, 1,4,5,7-tetrahydropyrano[3,4-b]pyrrolyl, 5,6-dihydro-4H-furo[3,2-b]pyrrolyl, 6,7-dihydro-5H-furo[3,2-b]pyranyl, 5,7-dihydro-4H-thieno[2,3-c]pyranyl, 2,3-dihydro-1H-pyrrolo[2,3-b]pyridinyl, 2,3-dihydrofuro[2,3-b]pyridinyl, 4,5,6,7-tetrahydro-1H-pyrrolo[2,3-b]pyridinyl, 4,5,6,7-tetrahydrofuro[3,2-c]pyridinyl, 4,5,6,7-tetrahydrothieno[3,2-b]pyridinyl, 1,2,3,4-tetrahydro-1,6-naphthyridinyl, and the like.

The term “aryl” refers to a radical of a monocyclic or polycyclic (e.g., bicyclic or tricyclic) 4n+2 aromatic ring system (e.g., having 6, 10, or 14, electrons shared in a cyclic array) having 6-14 ring carbon atoms and zero heteroatoms provided in the aromatic ring system (“C₆₋₁₄ aryl”). In some embodiments, an aryl group has 6 ring carbon atoms (“C₆ aryl”; e.g., phenyl). In some embodiments, an aryl group has 10 ring carbon atoms (“C₁₀ aryl”; e.g., naphthyl such as 1-naphthyl and 2-naphthyl). In some embodiments, an aryl group has 14 ring carbon atoms (“C₁₄ aryl”; e.g., anthracyl). “Aryl” also includes ring systems wherein the aryl ring, as defined above, is fused with one or more carbocyclyl or heterocyclyl groups wherein the radical or point of attachment is on the aryl ring, and in such instances, the number of carbon atoms continue to designate the number of carbon atoms in the aryl ring system. Unless otherwise specified, each instance of an aryl group is independently unsubstituted (an “unsubstituted aryl”) or substituted (a “substituted aryl”) with one or more substituents. In certain embodiments, the aryl group is an unsubstituted C₆₋₁₄ aryl. In certain embodiments, the aryl group is a substituted C₆₋₁₄ aryl.

“Aralkyl” is a subset of “alkyl” and refers to an alkyl group substituted by an aryl group, wherein the point of attachment is on the alkyl moiety.

The term “heteroaryl” refers to a radical of a 5-14 membered monocyclic or polycyclic (e.g., bicyclic, tricyclic) 4n+2 aromatic ring system (e.g., having 6, 10, or 14π electrons shared in a cyclic array) having ring carbon atoms and 1-4 ring heteroatoms provided in the aromatic ring system, wherein each heteroatom is independently selected from nitrogen, oxygen, and sulfur (“5-14 membered heteroaryl”). In heteroaryl groups that contain one or more nitrogen atoms, the point of attachment can be a carbon or nitrogen atom, as valency permits. Heteroaryl polycyclic ring systems can include one or more heteroatoms in one or both rings. “Heteroaryl” includes ring systems wherein the heteroaryl ring, as defined above, is fused with one or more carbocyclyl or heterocyclyl groups wherein the point of attachment is on the heteroaryl ring, and in such instances, the number of ring members continue to designate the number of ring members in the heteroaryl ring system. “Heteroaryl” also includes ring systems wherein the heteroaryl ring, as defined above, is fused with one or more aryl groups wherein the point of attachment is either on the aryl or heteroaryl ring, and in such instances, the number of ring members designates the number of ring members in the fused polycyclic (aryl/heteroaryl) ring system. Polycyclic heteroaryl groups wherein one ring does not contain a heteroatom (e.g., indolyl, quinolinyl, carbazolyl, and the like) the point of attachment can be on either ring, i.e., either the ring bearing a heteroatom (e.g., 2-indolyl) or the ring that does not contain a heteroatom (e.g., 5-indolyl). In certain embodiments, the heteroaryl is substituted or unsubstituted, 5- or 6-membered, monocyclic heteroaryl, wherein 1, 2, 3, or 4 atoms in the heteroaryl ring system are independently oxygen, nitrogen, or sulfur. In certain embodiments, the heteroaryl is substituted or unsubstituted, 9- or 10-membered, bicyclic heteroaryl, wherein 1, 2, 3, or 4 atoms in the heteroaryl ring system are independently oxygen, nitrogen, or sulfur.

In some embodiments, a heteroaryl group is a 5-10 membered aromatic ring system having ring carbon atoms and 1-4 ring heteroatoms provided in the aromatic ring system, wherein each heteroatom is independently selected from nitrogen, oxygen, and sulfur (“5-10 membered heteroaryl”). In some embodiments, a heteroaryl group is a 5-8 membered aromatic ring system having ring carbon atoms and 1-4 ring heteroatoms provided in the aromatic ring system, wherein each heteroatom is independently selected from nitrogen, oxygen, and sulfur (“5-8 membered heteroaryl”). In some embodiments, a heteroaryl group is a 5-6 membered aromatic ring system having ring carbon atoms and 1-4 ring heteroatoms provided in the aromatic ring system, wherein each heteroatom is independently selected from nitrogen, oxygen, and sulfur (“5-6 membered heteroaryl”). In some embodiments, the 5-6 membered heteroaryl has 1-3 ring heteroatoms selected from nitrogen, oxygen, and sulfur. In some embodiments, the 5-6 membered heteroaryl has 1-2 ring heteroatoms selected from nitrogen, oxygen, and sulfur. In some embodiments, the 5-6 membered heteroaryl has 1 ring heteroatom selected from nitrogen, oxygen, and sulfur. Unless otherwise specified, each instance of a heteroaryl group is independently unsubstituted (an “unsubstituted heteroaryl”) or substituted (a “substituted heteroaryl”) with one or more substituents. In certain embodiments, the heteroaryl group is an unsubstituted 5-14 membered heteroaryl. In certain embodiments, the heteroaryl group is a substituted 5-14 membered heteroaryl.

Exemplary 5-membered heteroaryl groups containing 1 heteroatom include, without limitation, pyrrolyl, furanyl, and thiophenyl. Exemplary 5-membered heteroaryl groups containing 2 heteroatoms include, without limitation, imidazolyl, pyrazolyl, oxazolyl, isoxazolyl, thiazolyl, and isothiazolyl. Exemplary 5-membered heteroaryl groups containing 3 heteroatoms include, without limitation, triazolyl, oxadiazolyl, and thiadiazolyl. Exemplary 5-membered heteroaryl groups containing 4 heteroatoms include, without limitation, tetrazolyl. Exemplary 6-membered heteroaryl groups containing 1 heteroatom include, without limitation, pyridinyl. Exemplary 6-membered heteroaryl groups containing 2 heteroatoms include, without limitation, pyridazinyl, pyrimidinyl, and pyrazinyl. Exemplary 6-membered heteroaryl groups containing 3 or 4 heteroatoms include, without limitation, triazinyl and tetrazinyl, respectively. Exemplary 7-membered heteroaryl groups containing 1 heteroatom include, without limitation, azepinyl, oxepinyl, and thiepinyl. Exemplary 5,6-bicyclic heteroaryl groups include, without limitation, indolyl, isoindolyl, indazolyl, benzotriazolyl, benzothiophenyl, isobenzothiophenyl, benzofuranyl, benzoisofuranyl, benzimidazolyl, benzoxazolyl, benzisoxazolyl, benzoxadiazolyl, benzthiazolyl, benzisothiazolyl, benzthiadiazolyl, indolizinyl, and purinyl. Exemplary 6,6-bicyclic heteroaryl groups include, without limitation, naphthyridinyl, pteridinyl, quinolinyl, isoquinolinyl, cinnolinyl, quinoxalinyl, phthalazinyl, and quinazolinyl. Exemplary tricyclic heteroaryl groups include, without limitation, phenanthridinyl, dibenzofuranyl, carbazolyl, acridinyl, phenothiazinyl, phenoxazinyl and phenazinyl.

“Heteroaralkyl” is a subset of “alkyl” and refers to an alkyl group substituted by a heteroaryl group, wherein the point of attachment is on the alkyl moiety.

The term “unsaturated bond” refers to a double or triple bond.

The term “unsaturated” or “partially unsaturated” refers to a moiety that includes at least one double or triple bond.

The term “saturated” refers to a moiety that does not contain a double or triple bond, i.e., the moiety only contains single bonds.

Affixing the suffix “-ene” to a group indicates the group is a divalent moiety, e.g., alkylene is the divalent moiety of alkyl, alkenylene is the divalent moiety of alkenyl, alkynylene is the divalent moiety of alkynyl, heteroalkylene is the divalent moiety of heteroalkyl, heteroalkenylene is the divalent moiety of heteroalkenyl, heteroalkynylene is the divalent moiety of heteroalkynyl, carbocyclylene is the divalent moiety of carbocyclyl, heterocyclylene is the divalent moiety of heterocyclyl, arylene is the divalent moiety of aryl, and heteroarylene is the divalent moiety of heteroaryl.

A group is optionally substituted unless expressly provided otherwise. The term “optionally substituted” refers to being substituted or unsubstituted. In certain embodiments, alkyl, alkenyl, alkynyl, heteroalkyl, heteroalkenyl, heteroalkynyl, carbocyclyl, heterocyclyl, aryl, and heteroaryl groups are optionally substituted. “Optionally substituted” refers to a group which may be substituted or unsubstituted (e.g., “substituted” or “unsubstituted” alkyl, “substituted” or “unsubstituted” alkenyl, “substituted” or “unsubstituted” alkynyl, “substituted” or “unsubstituted” heteroalkyl, “substituted” or “unsubstituted” heteroalkenyl, “substituted” or “unsubstituted” heteroalkynyl, “substituted” or “unsubstituted” carbocyclyl, “substituted” or “unsubstituted” heterocyclyl, “substituted” or “unsubstituted” aryl or “substituted” or “unsubstituted” heteroaryl group). In general, the term “substituted” means that at least one hydrogen present on a group is replaced with a permissible substituent, e.g., a substituent which upon substitution results in a stable compound, e.g., a compound which does not spontaneously undergo transformation such as by rearrangement, cyclization, elimination, or other reaction. Unless otherwise indicated, a “substituted” group has a substituent at one or more substitutable positions of the group, and when more than one position in any given structure is substituted, the substituent is either the same or different at each position. The term “substituted” is contemplated to include substitution with all permissible substituents of organic compounds, and includes any of the substituents described herein that results in the formation of a stable compound. The present invention contemplates any and all such combinations in order to arrive at a stable compound. For purposes of this invention, heteroatoms such as nitrogen may have hydrogen substituents and/or any suitable substituent as described herein which satisfy the valencies of the heteroatoms and results in the formation of a stable moiety. The invention is not intended to be limited in any manner by the exemplary substituents described herein.

Exemplary carbon atom substituents include, but are not limited to, halogen, —CN, —NO₂, —N₃, —SO₂H, —SO₃H, —OH, —OR^{aa}, —ON(R^{bb})₂, —N(R^{bb})₂, —N(R^{bb})₃⁺X⁻, —N(OR^{cc})R^{bb}, —SH, —SR^{aa}, —SSR^{cc}, —C(═O)R^{aa}, —CO₂H, —CHO, —C(OR^{cc})₂, —CO₂R^{aa}, —OC(═O)R^{aa}, —OCO₂R^{aa}, —C(═O)N(R^{bb})₂, —OC(═O)N(R^{bb})₂, —NR^{bb}C(═O)R^{aa}, —NR^{bb}CO₂R^{aa}, —NR^{bb}C(═O)N(R^{bb})₂, —C(═NR^{bb})R^{aa}, —C(═NR^{bb})OR^{aa}, —OC(═NR^{bb})R^{aa}, —OC(═NR^{bb})OR^{aa}, —C(═NR^{bb})N(R^{bb})₂, —OC(═NR^{bb})N(R^{bb})₂, —NR^{bb}C(═NR^{bb})N(R^{bb})₂, —C(═O)NR^{bb}SO₂R^{aa}, —NR^{bb}SO₂R^{aa}, —SO₂N(R^{bb})₂, —SO₂R^{aa}, —SO₂OR^{aa}, —OSO₂R^{aa}, —S(═O)R^{aa}, —OS(═O)R^{aa}, —Si(R^{aa})₃, —OSi(R^{aa})₃—C(═S)N(R^{bb})₂, —C(═O)SR^{aa}, —C(═S)SR^{aa}, —SC(═S)SR^{aa}, —SC(═O)SR^{aa}, —OC(═O)SR^{aa}, —SC(═O)OR^{aa}, —SC(═O)R^{aa}, —P(═O)₂R^{aa}, —OP(═O)₂R^{aa}, —P(═O)(R^{aa})₂, —OP(═O)(R^{aa})₂, —OP(═O)(OR^{cc})₂, —P(═O)₂N(R^{bb})₂, —OP(═O)₂N(R^{bb})₂, —P(═O)(NR^{bb})₂, —OP(═O)(NR^{bb})₂, —NR^{bb}P(═O)(OR^{cc})₂, —NR^{bb}P(═O)(NR^{bb})₂, —P(R^{cc})₂, —P(R^{cc})₃, —OP(R^{cc})₂, —OP(R^{cc})₃, —B(R^{aa})₂, —B(OR^{cc})₂, —BR^{aa}(OR^{cc}), C₁₋₁₀ alkyl, C₁₋₁₀ perhaloalkyl, C₂₋₁₀ alkenyl, C₂₋₁₀ alkynyl, heteroC₁₋₁₀ alkyl, heteroC₂₋₁₀alkenyl, heteroC₂₋₁₀alkynyl, C₃₋₁₀ carbocyclyl, 3-14 membered heterocyclyl, C₆₋₁₄ aryl, and 5-14 membered heteroaryl, wherein each alkyl, alkenyl, alkynyl, heteroalkyl, heteroalkenyl, heteroalkynyl, carbocyclyl, heterocyclyl, aryl, and heteroaryl is independently substituted with 0, 1, 2, 3, 4, or 5 R^{dd} groups;
or two geminal hydrogens on a carbon atom are replaced with the group ═O, ═S, ═NN(R^{bb})₂, ═NNR^{bb}C(═O)R^{aa}, ═NNR^{bb}C(═O)OR^{aa}, ═NNR^{bb}S(═O)₂R^{aa}, ═NR^{bb}, or ═NOR^{cc}.
each instance of R^{aa} is, independently, selected from C₁₋₁₀ alkyl, C₁₋₁₀ perhaloalkyl, C₂₋₁₀ alkenyl, C₂₋₁₀ alkynyl, heteroC₁₋₁₀ alkyl, heteroC₂₋₁₀alkenyl, heteroC₂₋₁₀alkynyl, C₃₋₁₀ carbocyclyl, 3-14 membered heterocyclyl, C₆₋₁₄ aryl, and 5-14 membered heteroaryl, or two R^{aa} groups are joined to form a 3-14 membered heterocyclyl or 5-14 membered heteroaryl ring, wherein each alkyl, alkenyl, alkynyl, heteroalkyl, heteroalkenyl, heteroalkynyl, carbocyclyl, heterocyclyl, aryl, and heteroaryl is independently substituted with 0, 1, 2, 3, 4, or 5 R^{dd} groups;
each instance of R^{bb} is, independently, selected from hydrogen, —OH, —OR^{aa}, —N(R^{cc})₂, —CN, —C(═O)R^{aa}, —C(═O)N(R^{cc})₂, —CO₂R^{aa}, —SO₂R^{aa}, —C(═NR^{cc})OR^{aa}, —C(═NR^{cc})N(R^{cc})₂, —SO₂N(R^{cc})₂, —SO₂R^{cc}, —SO₂OR^{cc}, —SOR^{aa}, —C(═S)N(R^{cc})₂, —C(═O)SR^{cc}, —C(═S)SR^{cc}, —P(═O)₂R^{aa}, —P(═O)(R^{aa})₂, —P(═O)₂N(R^{cc})₂, —P(═O)(NR^{cc})₂, C₁₋₁₀ alkyl, C₁₋₁₀ perhaloalkyl, C₂₋₁₀ alkenyl, C₂₋₁₀ alkynyl, heteroC₁₋₁₀alkyl, heteroC₂₋₁₀alkenyl, heteroC₂₋₁₀alkynyl, C₃₋₁₀ carbocyclyl, 3-14 membered heterocyclyl, C₆₋₁₄ aryl, and 5-14 membered heteroaryl, or two R^{bb} groups are joined to form a 3-14 membered heterocyclyl or 5-14 membered heteroaryl ring, wherein each alkyl, alkenyl, alkynyl, heteroalkyl, heteroalkenyl, heteroalkynyl, carbocyclyl, heterocyclyl, aryl, and heteroaryl is independently substituted with 0, 1, 2, 3, 4, or 5 R^{dd} groups;
each instance of R^{cc} is, independently, selected from hydrogen, C₁₋₁₀ alkyl, C₁₋₁₀ perhaloalkyl, C₂₋₁₀ alkenyl, C₂₋₁₀ alkynyl, heteroC₁₋₁₀ alkyl, heteroC₂₋₁₀alkenyl, heteroC₂₋₁₀alkynyl, C₃₋₁₀ carbocyclyl, 3-14 membered heterocyclyl, C₆₋₁₄ aryl, and 5-14 membered heteroaryl, or two R^{cc} groups are joined to form a 3-14 membered heterocyclyl or 5-14 membered heteroaryl ring, wherein each alkyl, alkenyl, alkynyl, heteroalkyl, heteroalkenyl, heteroalkynyl, carbocyclyl, heterocyclyl, aryl, and heteroaryl is independently substituted with 0, 1, 2, 3, 4, or 5 R^{dd} groups;
each instance of R^{dd} is, independently, selected from halogen, —CN, —NO₂, —N₃, —SO₂H, —SO₃H, —OH, —OR^{ee}, —ON(R^{ff})₂, —N(R^{ff})₂, —N(R^{ff})₃⁺X⁻, —N(OR^{ee})R^{ff}, —SH, —SR^{ee}, —SSR^{ee}, —C(═O)R^{ee}, —CO₂H, —CO₂R^{ee}, —OC(═O)R^{ee}, —OCO₂R^{ee}, —C(═O)N(R^{ff})₂, —OC(═O)N(R^{ff})₂, —NR^{ff}C(═O)R^{ee}, —NR^{ff}CO₂R^{ee}, —NR^{ff}C(═O)N(R^{ff})₂, —C(═NR^{ff})OR^{ee}, —OC(═NR^{ff})R^{ee}, —OC(═NR^{ff})OR^{ee}, —C(═NR^{ff})N(R^{ff})₂, —OC(═NR^{ff})N(R^{ff})₂, —NRC(═NR^{ff})N(Re)₂, —NR^{ff}SO₂R^{ee}, —SO₂N(R^{ff})₂, —SO₂R^{ee}, —SO₂OR^{ee}, —OSO₂R^{ee}, —S(═O)R^{ee}, —Si(R^{ee})₃, —OSi(R^{ee})₃, —C(═S)N(R^{ff})₂, —C(═O)SR^{ee}, —C(═S)SR^{ee}, —SC(═S)SR^{ee}, —P(═O)₂R^{ee}, —P(═O)(R^{ee})₂, —OP(═O)(R^{ee})₂, —OP(═O)(OR^{ee})₂, C₁₋₆ alkyl, C₁₋₆ perhaloalkyl, C₂₋₆ alkenyl, C₂₋₆ alkynyl, heteroC₁₋₆alkyl, heteroC₂₋₆alkenyl, heteroC₂₋₆alkynyl, C₃₋₁₀ carbocyclyl, 3-10 membered heterocyclyl, C₆₋₁₀ aryl, 5-10 membered heteroaryl, wherein each alkyl, alkenyl, alkynyl, heteroalkyl, heteroalkenyl, heteroalkynyl, carbocyclyl, heterocyclyl, aryl, and heteroaryl is independently substituted with 0, 1, 2, 3, 4, or 5 R^{gg} groups, or two geminal R^{dd} substituents can be joined to form O or ═S;
each instance of R^{ee} is, independently, selected from C₁₋₆ alkyl, C₁₋₆ perhaloalkyl, C₂₋₆ alkenyl, C₂₋₆ alkynyl, heteroC₁₋₆ alkyl, heteroC₂₋₆alkenyl, heteroC₂₋₆ alkynyl, C₃₋₁₀ carbocyclyl, C₆₋₁₀ aryl, 3-10 membered heterocyclyl, and 3-10 membered heteroaryl, wherein each alkyl, alkenyl, alkynyl, heteroalkyl, heteroalkenyl, heteroalkynyl, carbocyclyl, heterocyclyl, aryl, and heteroaryl is independently substituted with 0, 1, 2, 3, 4, or 5 R^{gg} groups;
each instance of R^{ff} is, independently, selected from hydrogen, C₁₋₆ alkyl, C₁₋₆ perhaloalkyl, C₂₋₆ alkenyl, C₂₋₆ alkynyl, heteroC₁₋₆alkyl, heteroC₂₋₆alkenyl, heteroC₂₋₆alkynyl, C₃₋₁₀ carbocyclyl, 3-10 membered heterocyclyl, C₆₋₁₀ aryl and 5-10 membered heteroaryl, or two R^{ff} groups are joined to form a 3-10 membered heterocyclyl or 5-10 membered heteroaryl ring, wherein each alkyl, alkenyl, alkynyl, heteroalkyl, heteroalkenyl, heteroalkynyl, carbocyclyl, heterocyclyl, aryl, and heteroaryl is independently substituted with 0, 1, 2, 3, 4, or 5 R^{gg} groups; and
each instance of R^{gg} is, independently, halogen, —CN, —NO₂, —N₃, —SO₂H, —SO₃H, —OH, —OC₁₋₆ alkyl, —ON(C₁₋₆ alkyl)₂, —N(C₁₋₆ alkyl)₂, —N(C₁₋₆ alkyl)₃⁺X⁻, —NH(C₁₋₆ alkyl)₂⁺X⁻, —NH₂(C₁₋₆ alkyl)⁺X⁻, —NH₃⁺X⁻, —N(OC₁₋₆ alkyl)(C₁₋₆ alkyl), —N(OH)(C₁₋₆ alkyl), —NH(OH), —SH, —SC₁₋₆ alkyl, —SS(C₁₋₆ alkyl), —C(═O)(C₁₋₆ alkyl), —CO₂H, —CO₂(C₁₋₆ alkyl), —OC(═O)(C₁₋₆ alkyl), —OCO₂(C₁₋₆ alkyl), —C(═O)NH₂, —C(═O)N(C₁₋₆ alkyl)₂, —OC(═O)NH(C₁₋₆ alkyl), —NHC(═O)(C₁₋₆ alkyl), —N(C₁₋₆ alkyl)C(═O)(C₁₋₆ alkyl), —NHCO₂(C₁₋₆ alkyl), —NHC(═O)N(C₁₋₆ alkyl)₂, —NHC(═O)NH(C₁₋₆ alkyl), —NHC(═O)NH₂, —C(═NH)O(C₁₋₆ alkyl), —OC(═NH)(C₁₋₆ alkyl), —OC(═NH)OC₁₋₆ alkyl, —C(═NH)N(C₁₋₆ alkyl)₂, —C(═NH)NH(C₁₋₆ alkyl), —C(═NH)NH₂, —OC(═NH)N(C₁₋₆ alkyl)₂, —OC(NH)NH(C₁₋₆ alkyl), —OC(NH)NH₂, —NHC(NH)N(C₁₋₆ alkyl)₂, —NHC(═NH)NH₂, —NHSO₂(C₁₋₆ alkyl), —SO₂N(C₁₋₆ alkyl)₂, —SO₂NH(C₁₋₆ alkyl), —SO₂NH₂, —SO₂C₁₋₆ alkyl, —SO₂OC₁₋₆ alkyl, —OSO₂C₁₋₆ alkyl, —SOC₁₋₆ alkyl, —Si(C₁₋₆ alkyl)₃, —OSi(C₁₋₆ alkyl)₃ —C(═S)N(C₁₋₆ alkyl)₂, C(═S)NH(C₁₋₆ alkyl), C(═S)NH₂, —C(═O)S(C₁₋₆ alkyl), —C(═S)SC₁₋₆ alkyl, —SC(═S)SC₁₋₆ alkyl, —P(═O)₂(C₁₋₆ alkyl), —P(═O)(C₁₋₆ alkyl)₂, —OP(═O)(C₁₋₆ alkyl)₂, —OP(═O)(OC₁₋₆ alkyl)₂, C₁₋₆ alkyl, C₁₋₆ perhaloalkyl, C₂₋₆ alkenyl, C₂₋₆ alkynyl, heteroC₁₋₆alkyl, heteroC₂₋₆alkenyl, heteroC₂₋₆alkynyl, C₃₋₁₀ carbocyclyl, C₆₋₁₀ aryl, 3-10 membered heterocyclyl, 5-10 membered heteroaryl; or two geminal R^{gg} substituents can be joined to form ═O or ═S; wherein X is a counterion.

In certain embodiments, the carbon atom substituents are independently halogen, substituted or unsubstituted C₁₋₆alkyl, —OR^{aa}, —SR^{aa}, —N(R^{bb})₂, —CN, —SCN, —NO₂, —C(═O)R^{aa}, —CO₂R^{aa}, —C(═O)N(R^{bb})₂, —OC(═O)R^{aa}, —OCO₂R^{aa}, —OC(═O)N(R^{bb})₂, —NR^{bb}C(═O)R^{aa}, —NR^{bb}CO₂R^{aa}, or —NR^{bb}C(═O)N(R^{bb})₂. In certain embodiments, the carbon atom substituents are independently halogen, substituted or unsubstituted C₁₋₆alkyl, —OR^{aa}, —SR^{aa}, —N(R^{bb})₂, —CN, —SCN, or —NO₂.

The term “halo” or “halogen” refers to fluorine (fluoro, —F), chlorine (chloro, —Cl), bromine (bromo, —Br), or iodine (iodo, —I).

The term “hydroxyl” or “hydroxy” refers to the group —OH. The term “substituted hydroxyl” or “substituted hydroxyl,” by extension, refers to a hydroxyl group wherein the oxygen atom directly attached to the parent molecule is substituted with a group other than hydrogen, and includes groups selected from —OR^{aa}, —ON(R^{bb})₂, —OC(═O)SR^{aa}, —OC(═O)R^{aa}, —OCO₂R^{aa}, —OC(═O)N(R^{bb})₂, —OC(═NR^{bb})R^{aa}, —OC(═NR^{bb})OR^{aa}, —OC(═NR^{bb})N(R^{bb})₂, —OS(═O)R^{aa}, —OSO₂R^{aa}, —OSi(R^{aa})₃, —OP(R^{cc})₂, —OP(R^{cc})₃, —OP(═O)₂R^{aa}, —OP(═O)(R^{aa})₂, —OP(═O)(OR^{cc})₂, —OP(═O)₂N(R^{bb})₂, and —OP(═O)(NR^{bb})₂, wherein R^{aa}, R^{bb}, and R^{cc} are as defined herein.

The term “thiol” or “thio” refers to the group —SH. The term “substituted thiol” or “substituted thio,” by extension, refers to a thiol group wherein the sulfur atom directly attached to the parent molecule is substituted with a group other than hydrogen, and includes groups selected from —SR^{aa}, —S═SR^{cc}, —SC(═S)SR^{aa}, —SC(═O)SR^{aa}, —SC(═O)OR^{aa}, and —SC(═O)R^{aa}, wherein R^{aa} and R^{cc} are as defined herein.

The term “amino” refers to the group —NH₂. The term “substituted amino,” by extension, refers to a monosubstituted amino, a disubstituted amino, or a trisubstituted amino. In certain embodiments, the “substituted amino” is a monosubstituted amino or a disubstituted amino group.

The term “monosubstituted amino” refers to an amino group wherein the nitrogen atom directly attached to the parent molecule is substituted with one hydrogen and one group other than hydrogen, and includes groups selected from —NH(R^{bb}), —NHC(═O)R^{aa}, —NHCO₂R^{aa}, —NHC(═O)N(R^{bb})₂, —NHC(═NR^{bb})N(R^{bb})₂, —NHSO₂R^{aa}, —NHP(═O)(OR^{cc})₂, and —NHP(═O)(NR^{bb})₂, wherein R^{aa}, R^{bb} and R^{cc} are as defined herein, and wherein R^{bb} of the group —NH(R^{bb}) is not hydrogen.

The term “disubstituted amino” refers to an amino group wherein the nitrogen atom directly attached to the parent molecule is substituted with two groups other than hydrogen, and includes groups selected from —N(R^{bb})₂, —NR^{bb} C(═O)R^{aa}, —NR^{bb}CO₂R^{aa}, —NR^{bb}C(═O)N(R^{bb})₂, —NR^{bb}C(═NR^{bb})N(R^{bb})₂, —NR^{bb}SO₂R^{aa}, —NR^{bb}P(═O)(OR_{cc})₂, and —NR^{bb}P(═O)(NR^{bb})₂, wherein R^{aa}, R^{bb}, and R^{cc} are as defined herein, with the proviso that the nitrogen atom directly attached to the parent molecule is not substituted with hydrogen.

The term “trisubstituted amino” refers to an amino group wherein the nitrogen atom directly attached to the parent molecule is substituted with three groups, and includes groups selected from —N(R^{bb})₃ and —N(R^{bb})₃⁺X⁻, wherein R^{bb} and X⁻ are as defined herein.

The term “sulfonyl” refers to a group selected from —SO₂N(R^{bb})₂, —SO₂R^{aa}, and —SO₂OR^{aa}, wherein R^{aa} and R^{bb} are as defined herein.

The term “sulfinyl” refers to the group —S(═O)R^{aa}, wherein R^{aa} is as defined herein.

The term “carbonyl” refers a group wherein the carbon directly attached to the parent molecule is sp² hybridized, and is substituted with an oxygen, nitrogen or sulfur atom, e.g., a group selected from ketones (—C(═O)R^{aa}), carboxylic acids (—CO₂H), aldehydes (—CHO), esters (—CO₂R^{aa}, —C(═O)SR^{aa}, —C(═S)SR^{aa}), amides (—C(═O)N(R^{bb})₂, —C(═O)NR^{bb}SO₂R^{aa}, —C(═S)N(R^{bb})₂), and imines (—C(═NR^{bb})R^{aa}, —C(═NR^{bb})OR^{aa}), —C(═NR^{bb})N(R^{bb})₂), wherein R^{aa} and R^{bb} are as defined herein.

The term “silyl” refers to the group —Si(R^{aa})₃, wherein R^{aa} is as defined herein.

The term “phosphoramido” refers to the group —O(P═O)(NR^{bb})₂, wherein each R^{bb} is as defined herein.

The term “oxo” refers to the group ═O, and the term “thiooxo” refers to the group ═S.

Nitrogen atoms can be substituted or unsubstituted as valency permits, and include primary, secondary, tertiary, and quaternary nitrogen atoms. Exemplary nitrogen atom substituents include, but are not limited to, hydrogen, —OH, —OR^{aa}, —N(R^{cc})₂, —CN, —C(═O)R^{aa}, —C(═O)N(R^{cc})₂, —CO₂R^{aa}, —SO₂R^{aa}, —C(═NR^{bb})R^{aa}, —C(═NR^{cc})OR^{aa}, —C(═NR^{cc})N(R^{cc})₂, —SO₂N(R^{cc})₂, —SO₂R^{cc}, —SO₂OR^{cc}, —SOR^{aa}, —C(═S)N(R^{cc})₂, —C(═O)SR^{cc}, —C(═S)SR^{cc}, —P(═O)₂R^{aa}, —P(═O)(R^{aa})₂, —P(═O)₂N(R^{cc})₂, —P(═O)(NR^{cc})₂, C₁₋₁₀ alkyl, C₁₋₁₀ perhaloalkyl, C₂₋₁₀ alkenyl, C₂₋₁₀ alkynyl, heteroC₁₋₁₀alkyl, heteroC₂₋₁₀alkenyl, heteroC₂₋₁₀alkynyl, C₃₋₁₀ carbocyclyl, 3-14 membered heterocyclyl, C₆₋₁₄ aryl, and 5-14 membered heteroaryl, or two R^{cc} groups attached to an N atom are joined to form a 3-14 membered heterocyclyl or 5-14 membered heteroaryl ring, wherein each alkyl, alkenyl, alkynyl, heteroalkyl, heteroalkenyl, heteroalkynyl, carbocyclyl, heterocyclyl, aryl, and heteroaryl is independently substituted with 0, 1, 2, 3, 4, or 5 R^{dd} groups, and wherein R^{aa}, R^{bb}, R^{cc} and R^{dd} are as defined above.

In certain embodiments, the substituent present on the nitrogen atom is an nitrogen protecting group (also referred to herein as an “amino protecting group”). Nitrogen protecting groups include, but are not limited to, —OH, —OR^{aa}, —N(R^{cc})₂, —C(═O)R^{aa}, —C(═O)N(R^{cc})₂, —CO₂R^{aa}, —SO₂R^{aa}, —C(═NR^{cc})R^{aa}, —C(═NR^{cc})OR^{aa}, —C(═NR^{cc})N(R^{cc})₂, —SO₂N(R^{cc})₂, —SO₂R^{cc}, —SO₂OR^{cc}, —SOR^{aa}, —C(═S)N(R^{cc})₂, —C(═O)SR^{cc}, —C(═S)SR^{cc}, C₁₋₁₀ alkyl (e.g., aralkyl, heteroaralkyl), C₂₋₁₀ alkenyl, C₂₋₁₀ alkynyl, heteroC₁₋₁₀ alkyl, heteroC₂₋₁₀alkenyl, heteroC₂₋₁₀ alkynyl, C₃₋₁₀ carbocyclyl, 3-14 membered heterocyclyl, C₆₋₁₄ aryl, and 5-14 membered heteroaryl groups, wherein each alkyl, alkenyl, alkynyl, heteroalkyl, heteroalkenyl, heteroalkynyl, carbocyclyl, heterocyclyl, aralkyl, aryl, and heteroaryl is independently substituted with 0, 1, 2, 3, 4, or 5 R^{dd} groups, and wherein R^{aa}, R^{bb}, R^{cc} and R^{dd} are as defined herein. Nitrogen protecting groups are well known in the art and include those described in detail in Protecting Groups in Organic Synthesis, T. W. Greene and P. G. M. Wuts, 3^{rd} edition, John Wiley & Sons, 1999, incorporated herein by reference.

For example, nitrogen protecting groups such as amide groups (e.g., —C(═O)R^{aa}) include, but are not limited to, formamide, acetamide, chloroacetamide, trichloroacetamide, trifluoroacetamide, phenylacetamide, 3-phenylpropanamide, picolinamide, 3-pyridylcarboxamide, N-benzoylphenylalanyl derivative, benzamide, p-phenylbenzamide, o-nitophenylacetamide, o-nitrophenoxyacetamide, acetoacetamide, (N′-dithiobenzyloxyacylamino)acetamide, 3-(p-hydroxyphenyl)propanamide, 3-(o-nitrophenyl)propanamide, 2-methyl-2-(o-nitrophenoxy)propanamide, 2-methyl-2-(o-phenylazophenoxy)propanamide, 4-chlorobutanamide, 3-methyl-3-nitrobutanamide, o-nitrocinnamide, N-acetylmethionine derivative, o-nitrobenzamide and o-(benzoyloxymethyl)benzamide.

Nitrogen protecting groups such as carbamate groups (e.g., —C(═O)OR^{aa}) include, but are not limited to, methyl carbamate, ethyl carbamante, 9-fluorenylmethyl carbamate (Fmoc), 9-(2-sulfo)fluorenylmethyl carbamate, 9-(2,7-dibromo)fluoroenylmethyl carbamate, 2,7-di-t-butyl-[9-(10,10-dioxo-10,10,10,10-tetrahydrothioxanthyl)]methyl carbamate (DBD-Tmoc), 4-methoxyphenacyl carbamate (Phenoc), 2,2,2-trichloroethyl carbamate (Troc), 2-trimethylsilylethyl carbamate (Teoc), 2-phenylethyl carbamate (hZ), 1-(1-adamantyl)-1-methylethyl carbamate (Adpoc), 1,1-dimethyl-2-haloethyl carbamate, 1,1-dimethyl-2,2-dibromoethyl carbamate (DB-t-BOC), 1,1-dimethyl-2,2,2-trichloroethyl carbamate (TCBOC), 1-methyl-1-(4-biphenylyl)ethyl carbamate (Bpoc), 1-(3,5-di-t-butylphenyl)-1-methylethyl carbamate (t-Bumeoc), 2-(2′- and 4′-pyridyl)ethyl carbamate (Pyoc), 2-(N,N-dicyclohexylcarboxamido)ethyl carbamate, t-butyl carbamate (BOC or Boc), 1-adamantyl carbamate (Adoc), vinyl carbamate (Voc), allyl carbamate (Alloc), 1-isopropylallyl carbamate (Ipaoc), cinnamyl carbamate (Coc), 4-nitrocinnamyl carbamate (Noc), 8-quinolyl carbamate, N-hydroxypiperidinyl carbamate, alkyldithio carbamate, benzyl carbamate (Cbz), p-methoxybenzyl carbamate (Moz), p-nitobenzyl carbamate, p-bromobenzyl carbamate, p-chlorobenzyl carbamate, 2,4-dichlorobenzyl carbamate, 4-methylsulfinylbenzyl carbamate (Msz), 9-anthrylmethyl carbamate, diphenylmethyl carbamate, 2-methylthioethyl carbamate, 2-methylsulfonylethyl carbamate, 2-(p-toluenesulfonyl)ethyl carbamate, [2-(1,3-dithianyl)]methyl carbamate (Dmoc), 4-methylthiophenyl carbamate (Mtpc), 2,4-dimethylthiophenyl carbamate (Bmpc), 2-phosphonioethyl carbamate (Peoc), 2-triphenylphosphonioisopropyl carbamate (Ppoc), 1,1-dimethyl-2-cyanoethyl carbamate, m-chloro-p-acyloxybenzyl carbamate, p-(dihydroxyboryl)benzyl carbamate, 5-benzisoxazolylmethyl carbamate, 2-(trifluoromethyl)-6-chromonylmethyl carbamate (Teroc), m-nitrophenyl carbamate, 3,5-dimethoxybenzyl carbamate, o-nitrobenzyl carbamate, 3,4-dimethoxy-6-nitrobenzyl carbamate, phenyl(o-nitrophenyl)methyl carbamate, t-amyl carbamate, S-benzyl thiocarbamate, p-cyanobenzyl carbamate, cyclobutyl carbamate, cyclohexyl carbamate, cyclopentyl carbamate, cyclopropylmethyl carbamate, p-decyloxybenzyl carbamate, 2,2-dimethoxyacylvinyl carbamate, o-(N,N-dimethylcarboxamido)benzyl carbamate, 1,1-dimethyl-3-(N,N-dimethylcarboxamido)propyl carbamate, 1,1-dimethylpropynyl carbamate, di(2-pyridyl)methyl carbamate, 2-furanylmethyl carbamate, 2-iodoethyl carbamate, isoborynl carbamate, isobutyl carbamate, isonicotinyl carbamate, p-(p′-methoxyphenylazo)benzyl carbamate, 1-methylcyclobutyl carbamate, 1-methylcyclohexyl carbamate, 1-methyl-1-cyclopropylmethyl carbamate, 1-methyl-1-(3,5-dimethoxyphenyl)ethyl carbamate, 1-methyl-1-(p-phenylazophenyl)ethyl carbamate, 1-methyl-1-phenylethyl carbamate, 1-methyl-1-(4-pyridyl)ethyl carbamate, phenyl carbamate, p-(phenylazo)benzyl carbamate, 2,4,6-tri-t-butylphenyl carbamate, 4-(trimethylammonium)benzyl carbamate, and 2,4,6-trimethylbenzyl carbamate.

Nitrogen protecting groups such as sulfonamide groups (e.g., —S(═O)₂R^{aa}) include, but are not limited to, p-toluenesulfonamide (Ts), benzenesulfonamide, 2,3,6,-trimethyl-4-methoxybenzenesulfonamide (Mtr), 2,4,6-trimethoxybenzenesulfonamide (Mtb), 2,6-dimethyl-4-methoxybenzenesulfonamide (Pme), 2,3,5,6-tetramethyl-4-methoxybenzenesulfonamide (Mte), 4-methoxybenzenesulfonamide (Mbs), 2,4,6-trimethylbenzenesulfonamide (Mts), 2,6-dimethoxy-4-methylbenzenesulfonamide (iMds), 2,2,5,7,8-pentamethylchroman-6-sulfonamide (Pmc), methanesulfonamide (Ms), β-trimethylsilylethanesulfonamide (SES), 9-anthracenesulfonamide, 4-(4′,8′-dimethoxynaphthylmethyl)benzenesulfonamide (DNMBS), benzylsulfonamide, trifluoromethylsulfonamide, and phenacylsulfonamide.

Other nitrogen protecting groups include, but are not limited to, phenothiazinyl-(10)-acyl derivative, N′-p-toluenesulfonylaminoacyl derivative, N′-phenylaminothioacyl derivative, N-benzoylphenylalanyl derivative, N-acetylmethionine derivative, 4,5-diphenyl-3-oxazolin-2-one, N-phthalimide, N-dithiasuccinimide (Dts), N-2,3-diphenylmaleimide, N-2,5-dimethylpyrrole, N-1,1,4,4-tetramethyldisilylazacyclopentane adduct (STABASE), 5-substituted 1,3-dimethyl-1,3,5-triazacyclohexan-2-one, 5-substituted 1,3-dibenzyl-1,3,5-triazacyclohexan-2-one, 1-substituted 3,5-dinitro-4-pyridone, N-methylamine, N-allylamine, N-[2-(trimethylsilyl)ethoxy]methylamine (SEM), N-3-acetoxypropylamine, N-(1-isopropyl-4-nitro-2-oxo-3-pyroolin-3-yl)amine, quaternary ammonium salts, N-benzylamine, N-di(4-methoxyphenyl)methylamine, N-5-dibenzosuberylamine, N-triphenylmethylamine (Tr), N-[(4-methoxyphenyl)diphenylmethyl]amine (MMTr), N-9-phenylfluorenylamine (PhF), N-2,7-dichloro-9-fluorenylmethyleneamine, N-ferrocenylmethylamino (Fcm), N-2-picolylamino N′-oxide, N-1,1-dimethylthiomethyleneamine, N-benzylideneamine, N-p-methoxybenzylideneamine, N-diphenylmethyleneamine, N-[(2-pyridyl)mesityl]methyleneamine, N—(N′,N′-dimethylaminomethylene)amine, N,N′-isopropylidenediamine, N-p-nitrobenzylideneamine, N-salicylideneamine, N-5-chlorosalicylideneamine, N-(5-chloro-2-hydroxyphenyl)phenylmethyleneamine, N-cyclohexylideneamine, N-(5,5-dimethyl-3-oxo-1-cyclohexenyl)amine, N-borane derivative, N-diphenylborinic acid derivative, N-[phenyl(pentaacylchromium- or tungsten)acyl]amine, N-copper chelate, N-zinc chelate, N-nitroamine, N-nitrosoamine, amine N-oxide, diphenylphosphinamide (Dpp), dimethylthiophosphinamide (Mpt), diphenylthiophosphinamide (Ppt), dialkyl phosphoramidates, dibenzyl phosphoramidate, diphenyl phosphoramidate, benzenesulfenamide, o-nitrobenzenesulfenamide (Nps), 2,4-dinitrobenzenesulfenamide, pentachlorobenzenesulfenamide, 2-nitro-4-methoxybenzenesulfenamide, triphenylmethylsulfenamide, and 3-nitropyridinesulfenamide (Npys).

In certain embodiments, the substituent present on an oxygen atom is an oxygen protecting group (also referred to herein as an “hydroxyl protecting group”). Oxygen protecting groups include, but are not limited to, —R^{aa}, —N(R^{bb})₂, —C(═O)SR^{aa}, —C(═O)R^{aa}, —CO₂R^{aa}, —C(═O)N(R^{bb})₂, —C(═NR^{bb})R^{aa}, —C(═NR^{bb})OR^{aa}, —C(═NR^{bb})N(R^{bb})₂, —S(═O)R^{aa}, —SO₂R^{aa}, —Si(R^{aa})₃, —P(R^{cc})₂, —P(R^{cc})₃, —P(═O)₂R^{aa}, —P(═O)(R^{aa})₂, —P(═O)(OR^{cc})₂, —P(═O)₂N(R^{bb})₂, and —P(═O)(NR^{bb})₂, wherein R^{aa}, R^{bb}, and R^{cc} are as defined herein. Oxygen protecting groups are well known in the art and include those described in detail in Protecting Groups in Organic Synthesis, T. W. Greene and P. G. M. Wuts, 3^{rd} edition, John Wiley & Sons, 1999, incorporated herein by reference.

Exemplary oxygen protecting groups include, but are not limited to, methyl, methoxylmethyl (MOM), methylthiomethyl (MTM), t-butylthiomethyl, (phenyldimethylsilyl)methoxymethyl (SMOM), benzyloxymethyl (BOM), p-methoxybenzyloxymethyl (PMBM), (4-methoxyphenoxy)methyl (p-AOM), guaiacolmethyl (GUM), t-butoxymethyl, 4-pentenyloxymethyl (POM), siloxymethyl, 2-methoxyethoxymethyl (MEM), 2,2,2-trichloroethoxymethyl, bis(2-chloroethoxy)methyl, 2-(trimethylsilyl)ethoxymethyl (SEMOR), tetrahydropyranyl (THP), 3-bromotetrahydropyranyl, tetrahydrothiopyranyl, 1-methoxycyclohexyl, 4-methoxytetrahydropyranyl (MTHP), 4-methoxytetrahydrothiopyranyl, 4-methoxytetrahydrothiopyranyl S,S-dioxide, 1-[(2-chloro-4-methyl)phenyl]-4-methoxypiperidin-4-yl (CTMP), 1,4-dioxan-2-yl, tetrahydrofuranyl, tetrahydrothiofuranyl, 2,3,3a,4,5,6,7,7a-octahydro-7,8,8-trimethyl-4,7-methanobenzofuran-2-yl, 1-ethoxyethyl, 1-(2-chloroethoxy)ethyl, 1-methyl-1-methoxyethyl, 1-methyl-1-benzyloxyethyl, 1-methyl-1-benzyloxy-2-fluoroethyl, 2,2,2-trichloroethyl, 2-trimethylsilylethyl, 2-(phenylselenyl)ethyl, t-butyl, allyl, p-chlorophenyl, p-methoxyphenyl, 2,4-dinitrophenyl, benzyl (Bn), p-methoxybenzyl, 3,4-dimethoxybenzyl, o-nitrobenzyl, p-nitrobenzyl, p-halobenzyl, 2,6-dichlorobenzyl, p-cyanobenzyl, p-phenylbenzyl, 2-picolyl, 4-picolyl, 3-methyl-2-picolyl N-oxido, diphenylmethyl, p,p′-dinitrobenzhydryl, 5-dibenzosuberyl, triphenylmethyl, α-naphthyldiphenylmethyl, p-methoxyphenyldiphenylmethyl, di(p-methoxyphenyl)phenylmethyl, tri(p-methoxyphenyl)methyl, 4-(4′-bromophenacyloxyphenyl)diphenylmethyl, 4,4′,4″-tris(4,5-dichlorophthalimidophenyl)methyl, 4,4′,4″-tris(levulinoyloxyphenyl)methyl, 4,4′,4″-tris(benzoyloxyphenyl)methyl, 3-(imidazol-1-yl)bis(4′,4″-dimethoxyphenyl)methyl, 1,1-bis(4-methoxyphenyl)-1′-pyrenylmethyl, 9-anthryl, 9-(9-phenyl)xanthenyl, 9-(9-phenyl-10-oxo)anthryl, 1,3-benzodithiolan-2-yl, benzisothiazolyl S,S-dioxido, trimethylsilyl (TMS), triethylsilyl (TES), triisopropylsilyl (TIPS), dimethylisopropylsilyl (IPDMS), diethylisopropylsilyl (DEIPS), dimethylthexylsilyl, t-butyldimethylsilyl (TBDMS), t-butyldiphenylsilyl (TBDPS), tribenzylsilyl, tri-p-xylylsilyl, triphenylsilyl, diphenylmethylsilyl (DPMS), t-butylmethoxyphenylsilyl (TBMPS), formate, benzoylformate, acetate, chloroacetate, dichloroacetate, trichloroacetate, trifluoroacetate, methoxyacetate, triphenylmethoxyacetate, phenoxyacetate, p-chlorophenoxyacetate, 3-phenylpropionate, 4-oxopentanoate (levulinate), 4,4-(ethylenedithio)pentanoate (levulinoyldithioacetal), pivaloate, adamantoate, crotonate, 4-methoxycrotonate, benzoate, p-phenylbenzoate, 2,4,6-trimethylbenzoate (mesitoate), methyl carbonate, 9-fluorenylmethyl carbonate (Fmoc), ethyl carbonate, 2,2,2-trichloroethyl carbonate (Troc), 2-(trimethylsilyl)ethyl carbonate (TMSEC), 2-(phenylsulfonyl) ethyl carbonate (Psec), 2-(triphenylphosphonio) ethyl carbonate (Peoc), isobutyl carbonate, vinyl carbonate, allyl carbonate, t-butyl carbonate (BOC or Boc), p-nitrophenyl carbonate, benzyl carbonate, p-methoxybenzyl carbonate, 3,4-dimethoxybenzyl carbonate, o-nitrobenzyl carbonate, p-nitrobenzyl carbonate, S-benzyl thiocarbonate, 4-ethoxy-1-napththyl carbonate, methyl dithiocarbonate, 2-iodobenzoate, 4-azidobutyrate, 4-nitro-4-methylpentanoate, o-(dibromomethyl)benzoate, 2-formylbenzenesulfonate, 2-(methylthiomethoxy)ethyl, 4-(methylthiomethoxy)butyrate, 2-(methylthiomethoxymethyl)benzoate, 2,6-dichloro-4-methylphenoxyacetate, 2,6-dichloro-4-(1,1,3,3-tetramethylbutyl)phenoxyacetate, 2,4-bis(1,1-dimethylpropyl)phenoxyacetate, chlorodiphenylacetate, isobutyrate, monosuccinoate, (E)-2-methyl-2-butenoate, o-(methoxyacyl)benzoate, α-naphthoate, nitrate, alkyl N,N,N′,N′-tetramethylphosphorodiamidate, alkyl N-phenylcarbamate, borate, dimethylphosphinothioyl, alkyl 2,4-dinitrophenylsulfenate, sulfate, methanesulfonate (mesylate), benzylsulfonate, and tosylate (Ts).

In certain embodiments, the substituent present on a sulfur atom is a sulfur protecting group (also referred to as a “thiol protecting group”). Sulfur protecting groups include, but are not limited to, —R^{aa}, —N(R^{bb})₂, —C(═O)SR^{aa}, —C(═O)R^{aa}, —CO₂R^{aa}, —C(═O)N(R^{bb})₂, —C(═NR^{bb})R^{aa}, —C(═NR^{bb})OR^{aa}, —C(═NR^{bb})N(R^{bb})₂, —S(═O)R^{aa}, —SO₂R^{aa}, —Si(R^{aa})₃, —P(R^{cc})₂, —P(R^{cc})₃, —P(═O)₂R^{aa}, —P(═O)(R^{aa})₂, —P(═O)(OR_{cc})₂, —P(═O)₂N(R^{bb})₂, and —P(═O)(NR^{bb})₂, wherein R^{aa}, R^{bb}, and R^{cc} are as defined herein. Sulfur protecting groups are well known in the art and include those described in detail in Protecting Groups in Organic Synthesis, T. W. Greene and P. G. M. Wuts, 3^{rd} edition, John Wiley & Sons, 1999, incorporated herein by reference.

The term “heteroatom” refers to an atom that is not hydrogen or carbon. In certain embodiments, the heteroatom is nitrogen. In certain embodiments, the heteroatom is oxygen. In certain embodiments, the heteroatom is sulfur.

A “counterion” or “anionic counterion” is a negatively charged group associated with a positively charged group in order to maintain electronic neutrality. An anionic counterion may be monovalent (i.e., including one formal negative charge). An anionic counterion may also be multivalent (i.e., including more than one formal negative charge), such as divalent or trivalent. Exemplary counterions include halide ions (e.g., F⁻, Cl⁻, Br⁻, I⁻), NO₃⁻, ClO₄⁻, OH⁻, H₂PO₄⁻, HCO₃⁻, HSO₄⁻, sulfonate ions (e.g., methansulfonate, trifluoromethanesulfonate, p-toluenesulfonate, benzenesulfonate, 10-camphor sulfonate, naphthalene-2-sulfonate, naphthalene-1-sulfonic acid-5-sulfonate, ethan-1-sulfonic acid-2-sulfonate, and the like), carboxylate ions (e.g., acetate, propanoate, benzoate, glycerate, lactate, tartrate, glycolate, gluconate, and the like), BF₄⁻, PF₄⁻, PF₆⁻, AsF₆⁻, SbF₆⁻, B[3,5-(CF₃)₂C₆H₃]₄]⁻, B(C₆F₅)₄⁻, BPh₄⁻, Al(OC(CF₃)₃)₄⁻, and carborane anions (e.g., CB₁₁H₁₂ or (HCB₁₁Me₅Br₆)⁻). Exemplary counterions which may be multivalent include CO₃²⁻, HPO₄²⁻, PO₄³⁻, B₄O₇²⁻, SO₄²⁻, S₂O₃²⁻, carboxylate anions (e.g., tartrate, citrate, fumarate, maleate, malate, malonate, gluconate, succinate, glutarate, adipate, pimelate, suberate, azelate, sebacate, salicylate, phthalates, aspartate, glutamate, and the like), and carboranes.

The term “solvate” refers to forms of the compound, or a salt thereof, that are associated with a solvent, usually by a solvolysis reaction. This physical association may include hydrogen bonding. Conventional solvents include water, methanol, ethanol, acetic acid, DMSO, THF, diethyl ether, and the like. The compounds described herein may be prepared, e.g., in crystalline form, and may be solvated. Suitable solvates include pharmaceutically acceptable solvates and further include both stoichiometric solvates and non-stoichiometric solvates. In certain instances, the solvate will be capable of isolation, for example, when one or more solvent molecules are incorporated in the crystal lattice of a crystalline solid. “Solvate” encompasses both solution-phase and isolatable solvates. Representative solvates include hydrates, ethanolates, and methanolates.

The term “tautomers” or “tautomeric” refers to two or more interconvertible compounds resulting from at least one formal migration of a hydrogen atom and at least one change in valency (e.g., a single bond to a double bond, a triple bond to a single bond, or vice versa). The exact ratio of the tautomers depends on several factors, including temperature, solvent, and pH. Tautomerizations (i.e., the reaction providing a tautomeric pair) may catalyzed by acid or base. Exemplary tautomerizations include keto-to-enol, amide-to-imide, lactam-to-lactim, enamine-to-imine, and enamine-to-(a different enamine) tautomerizations.

It is also to be understood that compounds that have the same molecular formula but differ in the nature or sequence of bonding of their atoms or the arrangement of their atoms in space are termed “isomers”. Isomers that differ in the arrangement of their atoms in space are termed “stereoisomers”.

Stereoisomers that are not mirror images of one another are termed “diastereomers” and those that are non-superimposable mirror images of each other are termed “enantiomers”. When a compound has an asymmetric center, for example, it is bonded to four different groups, a pair of enantiomers is possible. An enantiomer can be characterized by the absolute configuration of its asymmetric center and is described by the R- and S-sequencing rules of Cahn and Prelog, or by the manner in which the molecule rotates the plane of polarized light and designated as dextrorotatory or levorotatory (i.e., as (+) or (−)-isomers respectively). A chiral compound can exist as either individual enantiomer or as a mixture thereof. A mixture containing equal proportions of the enantiomers is called a “racemic mixture”.

The term “administer,” “administering,” or “administration” refers to implanting, absorbing, ingesting, injecting, inhaling, or otherwise introducing a compound or cell described herein or generated as described herein, or a composition thereof, in or on a subject.

The terms “treatment,” “treat,” and “treating” refer to reversing, alleviating, delaying the onset of, or inhibiting the progress of a disease. In some embodiments, treatment may be administered after one or more signs or symptoms of the disease have developed or have been observed. In other embodiments, treatment may be administered in the absence of signs or symptoms of the disease. For example, treatment may be administered to a susceptible subject prior to the onset of symptoms (e.g., in light of a history of symptoms and/or in light of exposure to a pathogen and/or in light of detecting that the subject has a genotype associated with the disease). Treatment may also be continued after symptoms have resolved, for example, to delay or prevent recurrence.

The terms “condition,” “disease,” and “disorder” are used interchangeably.

As used herein, the term “salt” refers to any and all salts, and encompasses pharmaceutically acceptable salts.

As used herein, the term “pharmaceutically acceptable salt” refers to those salts which are, within the scope of sound medical judgment, suitable for use in contact with the tissues of humans and lower animals without undue toxicity, irritation, allergic response and the like, and are commensurate with a reasonable benefit/risk ratio. Pharmaceutically acceptable salts are well known in the art. For example, Berge et al., describe pharmaceutically acceptable salts in detail in J. Pharmaceutical Sciences, 1977, 66, 1-19, incorporated herein by reference. Pharmaceutically acceptable salts of the compounds of this invention include those derived from suitable inorganic and organic acids and bases. Examples of pharmaceutically acceptable, nontoxic acid addition salts are salts of an amino group formed with inorganic acids such as hydrochloric acid, hydrobromic acid, phosphoric acid, sulfuric acid, and perchloric acid or with organic acids such as acetic acid, oxalic acid, maleic acid, tartaric acid, citric acid, succinic acid, or malonic acid or by using other methods known in the art such as ion exchange. Other pharmaceutically acceptable salts include adipate, alginate, ascorbate, aspartate, benzenesulfonate, benzoate, bisulfate, borate, butyrate, camphorate, camphorsulfonate, citrate, cyclopentanepropionate, digluconate, dodecylsulfate, ethanesulfonate, formate, fumarate, glucoheptonate, glycerophosphate, gluconate, hemisulfate, heptanoate, hexanoate, hydroiodide, 2-hydroxy-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 3-phenylpropionate, phosphate, picrate, pivalate, propionate, stearate, succinate, sulfate, tartrate, thiocyanate, p-toluenesulfonate, undecanoate, valerate salts, and the like. Salts derived from appropriate bases include alkali metal, alkaline earth metal, ammonium and N⁺(C₁₋₄ alkyl)₄⁻ salts. Representative alkali or alkaline earth metal salts include sodium, lithium, potassium, calcium, magnesium, and the like. Further pharmaceutically acceptable salts include, when appropriate, nontoxic ammonium, quaternary ammonium, and amine cations formed using counterions such as halide, hydroxide, carboxylate, sulfate, phosphate, nitrate, lower alkyl sulfonate, and aryl sulfonate.

The term “small molecule” refers to molecules, whether naturally-occurring or artificially created (e.g., via chemical synthesis) that have a relatively low molecular weight. Typically, a small molecule is an organic compound (i.e., it contains carbon). The small molecule may contain multiple carbon-carbon bonds, stereocenters, and other functional groups (e.g., amines, hydroxyl, carbonyls, and heterocyclic rings, etc.). In certain embodiments, the molecular weight of a small molecule is not more than about 1,000 g/mol, not more than about 900 g/mol, not more than about 800 g/mol, not more than about 700 g/mol, not more than about 600 g/mol, not more than about 500 g/mol, not more than about 400 g/mol, not more than about 300 g/mol, not more than about 200 g/mol, or not more than about 100 g/mol. In certain embodiments, the molecular weight of a small molecule is at least about 100 g/mol, at least about 200 g/mol, at least about 300 g/mol, at least about 400 g/mol, at least about 500 g/mol, at least about 600 g/mol, at least about 700 g/mol, at least about 800 g/mol, or at least about 900 g/mol, or at least about 1,000 g/mol. Combinations of the above ranges (e.g., at least about 200 g/mol and not more than about 500 g/mol) are also possible. In certain embodiments, the small molecule is a therapeutically active agent such as a drug (e.g., a molecule approved by the U.S. Food and Drug Administration as provided in the Code of Federal Regulations (C.F.R.)). The small molecule may also be complexed with one or more metal atoms and/or metal ions. In this instance, the small molecule is also referred to as a “small organometallic molecule.” Preferred small molecules are biologically active in that they produce a biological effect in animals, preferably mammals, more preferably humans. Small molecules include, but are not limited to, radionuclides and imaging agents. In certain embodiments, the small molecule is a drug. Preferably, though not necessarily, the drug is one that has already been deemed safe and effective for use in humans or animals by the appropriate governmental agency or regulatory body. For example, drugs approved for human use are listed by the FDA under 21 C.F.R. §§ 330.5, 331 through 361, and 440 through 460, incorporated herein by reference; drugs for veterinary use are listed by the FDA under 21 C.F.R. §§ 500 through 589, incorporated herein by reference. All listed drugs are considered acceptable for use in accordance with the present invention.

The term “inhibition,” “inhibiting,” “inhibit,” or “inhibitor” refers to the ability of a compound to reduce, slow, halt or prevent activity of a particular biological process (e.g., kinase activity) in a cell.

The term “gene” refers to a nucleic acid fragment that provides a template that can be used for producing a gene product. In certain embodiments, the nucleic acid fragment includes regulatory sequences preceding and following the coding sequence. “Native gene” refers to a gene as found in nature with its own regulatory sequences. “Chimeric gene” or “chimeric construct” refers to any gene or a construct, not a native gene, comprising regulatory and coding sequences that are not found together in nature. Accordingly, a chimeric gene or chimeric construct may comprise regulatory sequences and coding sequences that are derived from different sources, or regulatory sequences and coding sequences derived from the same source, but arranged in a manner different than that found in nature. “Endogenous gene” refers to a native gene in its natural location in the genome of an organism. A “foreign” gene refers to a gene not normally found in the host organism, but which is introduced into the host organism by gene transfer. Foreign genes can comprise native genes inserted into a non-native organism, or chimeric genes. A “transgene” is a gene that has been introduced into the genome by a transformation procedure.

The term “gene product” (also referred to herein as “gene expression product” or “expression product”) encompasses products resulting from expression of a gene, such as RNA transcribed from a gene and polypeptides arising from translation of such RNA. It will be appreciated that certain gene products may undergo processing or modification, e.g., in a cell. For example, RNA transcripts may be spliced, polyadenylated, etc., prior to mRNA translation, and/or polypeptides may undergo co-translational or post-translational processing such as removal of secretion signal sequences, removal of organelle targeting sequences, or modifications such as phosphorylation, fatty acylation, etc. The term “gene product” encompasses such processed or modified forms. Genomic, mRNA, polypeptide sequences from a variety of species, including human, are known in the art and are available in publicly accessible databases such as those available at the National Center for Biotechnology Information (www.ncbi.nih.gov) or Universal Protein Resource (www.uniprot.org). Databases include, e.g., GenBank, RefSeq, Gene, UniProtKB/SwissProt, UniProtKB/Trembl, and the like. In general, sequences, e.g., mRNA and polypeptide sequences, in the NCBI Reference Sequence database may be used as gene product sequences for a gene of interest. It will be appreciated that multiple alleles of a gene may exist among individuals of the same species. For example, differences in one or more nucleotides (e.g., up to about 1%, 2%, 3-5% of the nucleotides) of the nucleic acids encoding a particular protein may exist among individuals of a given species. Due to the degeneracy of the genetic code, such variations often do not alter the encoded amino acid sequence, although DNA polymorphisms that lead to changes in the sequence of the encoded proteins can exist. Examples of polymorphic variants can be found in, e.g., the Single Nucleotide Polymorphism Database (dbSNP), available at the NCBI website at www.ncbi.nlm.nih.gov/projects/SNP/. (Sherry S T, et al. (2001). “dbSNP: the NCBI database of genetic variation”. Nucleic Acids Res. 29 (1): 308-311; Kitts A, and Sherry S, (2009). The single nucleotide polymorphism database (dbSNP) of nucleotide sequence variation in The NCBI Handbook [Internet]. McEntyre J, Ostell J, editors. Bethesda (MD): National Center for Biotechnology Information (US); 2002 (www.ncbi.nlm.nih.gov/bookshelf/br.fcgi?book=handbook&part=ch5). Multiple isoforms of certain proteins may exist, e.g., as a result of alternative RNA splicing or editing. In general, where aspects of this disclosure pertain to a gene or gene product, embodiments pertaining to allelic variants or isoforms are encompassed, if applicable, unless indicated otherwise. Certain embodiments may be directed to particular sequence(s), e.g., particular allele(s) or isoform(s).

The term “purified protein” or “purified enzyme” refers to a protein or enzyme that is greater than or equal to 95% pure. In certain embodiments, a purified enzyme refers to a protein or enzyme that is greater than 96% pure. In certain embodiments, a purified enzyme refers to a protein or enzyme that is greater than 97% pure. In certain embodiments, a purified enzyme refers to a protein or enzyme that is greater than 98% pure. In certain embodiments, a purified enzyme refers to a protein or enzyme that is greater than 99% pure. In certain embodiments, a purified enzyme refers to a protein or enzyme that is 100% pure. Protein purification is a series of processes intended to isolate one or a few proteins from a complex mixture, usually cells, tissues or whole organisms. Protein purification is vital for the characterization of the function, structure and interactions of the protein of interest. The purification process may separate the protein and non-protein parts of the mixture, and finally separate the desired protein from all other proteins. Separation of one protein from all others is typically the most laborious aspect of protein purification. Separation steps usually exploit differences in protein size, physico-chemical properties, binding affinity and biological activity. The term “partially purified protein” or “partially purified enzyme” refers to a protein or enzyme that is less than 95% pure. The term “unpurified protein” or “unpurified enzyme” refers to a protein or enzyme that has not undergone protein purification.

The term “neurological disease” refers to any disease of the nervous system, including diseases that involve the central nervous system (brain, brainstem and cerebellum), the peripheral nervous system (including cranial nerves), and the autonomic nervous system (parts of which are located in both central and peripheral nervous system). Neurodegenerative diseases refer to a type of neurological disease marked by the loss of nerve cells, including, but not limited to, Alzheimer's disease, Parkinson's disease, amyotrophic lateral sclerosis, tauopathies (including frontotemporal dementia), and Huntington's disease. Examples of neurological diseases include, but are not limited to, headache, stupor and coma, dementia, seizure, sleep disorders, trauma, infections, neoplasms, neuro-ophthalmologic disorders, movement disorders, demyelinating diseases, spinal cord disorders, and disorders of peripheral nerves, muscle and neuromuscular junctions. Addiction and mental illness, include, but are not limited to, bipolar disorder, depression, and schizophrenia, and are also included in the definition of neurological diseases. Examples of neurological diseases include acquired epileptiform aphasia; acute disseminated encephalomyelitis; adrenoleukodystrophy; agenesis of the corpus callosum; agnosia; Aicardi syndrome; Alexander disease; Alpers' disease; alternating hemiplegia; Alzheimer's disease; amyotrophic lateral sclerosis; anencephaly; Angelman syndrome; angiomatosis; anoxia; aphasia; apraxia; arachnoid cysts; arachnoiditis; Arnold-Chiari malformation; arteriovenous malformation; Asperger syndrome; ataxia telangiectasia; attention deficit hyperactivity disorder; autism; autonomic dysfunction; back pain; Batten disease; Behcet's disease; Bell's palsy; benign essential blepharospasm; benign focal; amyotrophy; benign intracranial hypertension; Binswanger's disease; blepharospasm; Bloch Sulzberger syndrome; brachial plexus injury; brain abscess; brain injury; brain tumors (including glioblastoma multiforme); spinal tumor; Brown-Sequard syndrome; Canavan disease; carpal tunnel syndrome (CTS); causalgia; central pain syndrome; central pontine myelinolysis; cephalic disorder; cerebral aneurysm; cerebral arteriosclerosis; cerebral atrophy; cerebral gigantism; cerebral palsy; Charcot-Marie-Tooth disease; chemotherapy-induced neuropathy and neuropathic pain; Chiari malformation; chorea; chronic inflammatory demyelinating polyneuropathy (CIDP); chronic pain; chronic regional pain syndrome; Coffin Lowry syndrome; coma, including persistent vegetative state; congenital facial diplegia; corticobasal degeneration; cranial arteritis; craniosynostosis; Creutzfeldt-Jakob disease; cumulative trauma disorders; Cushing's syndrome; cytomegalic inclusion body disease (CIBD); cytomegalovirus infection; dancing eyes-dancing feet syndrome; Dandy-Walker syndrome; Dawson disease; De Morsier's syndrome; Dejerine-Klumpke palsy; dementia; dermatomyositis; diabetic neuropathy; diffuse sclerosis; dysautonomia; dysgraphia; dyslexia; dystonias; early infantile epileptic encephalopathy; empty sella syndrome; encephalitis; encephaloceles; encephalotrigeminal angiomatosis; epilepsy; Erb's palsy; essential tremor; Fabry's disease; Fahr's syndrome; fainting; familial spastic paralysis; febrile seizures; Fisher syndrome; Friedreich's ataxia; frontotemporal dementia and other “tauopathies”; Gaucher's disease; Gerstmann's syndrome; giant cell arteritis; giant cell inclusion disease; globoid cell leukodystrophy; Guillain-Barre syndrome; HTLV-1 associated myelopathy; Hallervorden-Spatz disease; head injury; headache; hemifacial spasm; hereditary spastic paraplegia; heredopathia atactica polyneuritiformis; herpes zoster oticus; herpes zoster; Hirayama syndrome; HIV-associated dementia and neuropathy (see also neurological manifestations of AIDS); holoprosencephaly; Huntington's disease and other polyglutamine repeat diseases; hydranencephaly; hydrocephalus; hypercortisolism; hypoxia; immune-mediated encephalomyelitis; inclusion body myositis; incontinentia pigmenti; infantile; phytanic acid storage disease; Infantile Refsum disease; infantile spasms; inflammatory myopathy; intracranial cyst; intracranial hypertension; Joubert syndrome; Kearns-Sayre syndrome; Kennedy disease; Kinsbourne syndrome; Klippel Feil syndrome; Krabbe disease; Kugelberg-Welander disease; kuru; Lafora disease; Lambert-Eaton myasthenic syndrome; Landau-Kleffner syndrome; lateral medullary (Wallenberg) syndrome; learning disabilities; Leigh's disease; Lennox-Gastaut syndrome; Lesch-Nyhan syndrome; leukodystrophy; Lewy body dementia; lissencephaly; locked-in syndrome; Lou Gehrig's disease (aka motor neuron disease or amyotrophic lateral sclerosis); lumbar disc disease; lyme disease-neurological sequelae; Machado-Joseph disease; macrencephaly; megalencephaly; Melkersson-Rosenthal syndrome; Menieres disease; meningitis; Menkes disease; metachromatic leukodystrophy; microcephaly; migraine; Miller Fisher syndrome; mini-strokes; mitochondrial myopathies; Mobius syndrome; monomelic amyotrophy; motor neurone disease; moyamoya disease; mucopolysaccharidoses; multi-infarct dementia; multifocal motor neuropathy; multiple sclerosis and other demyelinating disorders; multiple system atrophy with postural hypotension; muscular dystrophy; myasthenia gravis; myelinoclastic diffuse sclerosis; myoclonic encephalopathy of infants; myoclonus; myopathy; myotonia congenital; narcolepsy; neurofibromatosis; neuroleptic malignant syndrome; neurological manifestations of AIDS; neurological sequelae of lupus; neuromyotonia; neuronal ceroid lipofuscinosis; neuronal migration disorders; Niemann-Pick disease; O'Sullivan-McLeod syndrome; occipital neuralgia; occult spinal dysraphism sequence; Ohtahara syndrome; olivopontocerebellar atrophy; opsoclonus myoclonus; optic neuritis; orthostatic hypotension; overuse syndrome; paresthesia; Parkinson's disease; paramyotonia congenita; paraneoplastic diseases; paroxysmal attacks; Parry Romberg syndrome; Pelizaeus-Merzbacher disease; periodic paralyses; peripheral neuropathy; painful neuropathy and neuropathic pain; persistent vegetative state; pervasive developmental disorders; photic sneeze reflex; phytanic acid storage disease; Pick's disease; pinched nerve; pituitary tumors; polymyositis; porencephaly; Post-Polio syndrome; postherpetic neuralgia (PHN); postinfectious encephalomyelitis; postural hypotension; Prader-Willi syndrome; primary lateral sclerosis; prion diseases; progressive; hemifacial atrophy; progressive multifocal leukoencephalopathy; progressive sclerosing poliodystrophy; progressive supranuclear palsy; pseudotumor cerebri; Ramsay-Hunt syndrome (Type I and Type II); Rasmussen's Encephalitis; reflex sympathetic dystrophy syndrome; Refsum disease; repetitive motion disorders; repetitive stress injuries; restless legs syndrome; retrovirus-associated myelopathy; Rett syndrome; Reye's syndrome; Saint Vitus Dance; Sandhoff disease; Schilder's disease; schizencephaly; septo-optic dysplasia; shaken baby syndrome; shingles; Shy-Drager syndrome; Sjogren's syndrome; sleep apnea; Soto's syndrome; spasticity; spina bifida; spinal cord injury; spinal cord tumors; spinal muscular atrophy; stiff-person syndrome; stroke; Sturge-Weber syndrome; subacute sclerosing panencephalitis; subarachnoid hemorrhage; subcortical arteriosclerotic encephalopathy; sydenham chorea; syncope; syringomyelia; tardive dyskinesia; Tay-Sachs disease; temporal arteritis; tethered spinal cord syndrome; Thomsen disease; thoracic outlet syndrome; tic douloureux; Todd's paralysis; Tourette syndrome; transient ischemic attack; transmissible spongiform encephalopathies; transverse myelitis; traumatic brain injury; tremor; trigeminal neuralgia; tropical spastic paraparesis; tuberous sclerosis; vascular dementia (multi-infarct dementia); vasculitis including temporal arteritis; Von Hippel-Lindau Disease (VHL); Wallenberg's syndrome; Werdnig-Hoffman disease; West syndrome; whiplash; Williams syndrome; Wilson's disease; and Zellweger syndrome.

The term “psychiatric disorder” refers to a disease of the mind and includes diseases and disorders listed in the Diagnostic and Statistical Manual of Mental Disorders—Fourth Edition (DSM-IV), published by the American Psychiatric Association, Washington D. C. (1994). Psychiatric disorders include, but are not limited to, anxiety disorders (e.g., acute stress disorder agoraphobia, generalized anxiety disorder, obsessive-compulsive disorder, panic disorder, posttraumatic stress disorder, separation anxiety disorder, social phobia, and specific phobia), childhood disorders, (e.g., attention-deficit/hyperactivity disorder, conduct disorder, and oppositional defiant disorder), eating disorders (e.g., anorexia nervosa and bulimia nervosa), mood disorders (e.g., depression, bipolar disorder, cyclothymic disorder, dysthymic disorder, and major depressive disorder), personality disorders (e.g., antisocial personality disorder, avoidant personality disorder, borderline personality disorder, dependent personality disorder, histrionic personality disorder, narcissistic personality disorder, obsessive-compulsive personality disorder, paranoid personality disorder, schizoid personality disorder, and schizotypal personality disorder), psychotic disorders (e.g., brief psychotic disorder, delusional disorder, schizoaffective disorder, schizophreniform disorder, schizophrenia, and shared psychotic disorder), substance-related disorders (e.g., alcohol dependence, amphetamine dependence, cannabis dependence, cocaine dependence, hallucinogen dependence, inhalant dependence, nicotine dependence, opioid dependence, phencyclidine dependence, and sedative dependence), adjustment disorder, autism, delirium, dementia, multi-infarct dementia, learning and memory disorders (e.g., amnesia and age-related memory loss), and Tourette's disorder.

The term “cancer” refers to a class of diseases characterized by the development of abnormal cells that proliferate uncontrollably and have the ability to infiltrate and destroy normal body tissues. See, e.g., Stedman's Medical Dictionary, 25th ed.; Hensyl ed.; Williams & Wilkins: Philadelphia, 1990. Exemplary cancers include, but are not limited to, acoustic neuroma; adenocarcinoma; adrenal gland cancer; anal cancer; angiosarcoma (e.g., lymphangiosarcoma, lymphangioendotheliosarcoma, hemangiosarcoma); appendix cancer; benign monoclonal gammopathy; biliary cancer (e.g., cholangiocarcinoma); bladder cancer; breast cancer (e.g., adenocarcinoma of the breast, papillary carcinoma of the breast, mammary cancer, medullary carcinoma of the breast); brain cancer (e.g., meningioma, glioblastomas, glioma (e.g., astrocytoma, oligodendroglioma), medulloblastoma); bronchus cancer; carcinoid tumor; cervical cancer (e.g., cervical adenocarcinoma); choriocarcinoma; chordoma; craniopharyngioma; colorectal cancer (e.g., colon cancer, rectal cancer, colorectal adenocarcinoma); connective tissue cancer; epithelial carcinoma; ependymoma; endotheliosarcoma (e.g., Kaposi's sarcoma, multiple idiopathic hemorrhagic sarcoma); endometrial cancer (e.g., uterine cancer, uterine sarcoma); esophageal cancer (e.g., adenocarcinoma of the esophagus, Barrett's adenocarcinoma); Ewing's sarcoma; ocular cancer (e.g., intraocular melanoma, retinoblastoma); familiar hypereosinophilia; gall bladder cancer; gastric cancer (e.g., stomach adenocarcinoma); gastrointestinal stromal tumor (GIST); germ cell cancer; head and neck cancer (e.g., head and neck squamous cell carcinoma, oral cancer (e.g., oral squamous cell carcinoma), throat cancer (e.g., laryngeal cancer, pharyngeal cancer, nasopharyngeal cancer, oropharyngeal cancer)); hematopoietic cancers (e.g., leukemia such as acute lymphocytic leukemia (ALL) (e.g., B-cell ALL, T-cell ALL), acute myelocytic leukemia (AML) (e.g., B-cell AML, T-cell AML), chronic myelocytic leukemia (CML) (e.g., B-cell CML, T-cell CML), and chronic lymphocytic leukemia (CLL) (e.g., B-cell CLL, T-cell CLL)); lymphoma such as Hodgkin lymphoma (HL) (e.g., B-cell HL, T-cell HL) and non-Hodgkin lymphoma (NHL) (e.g., B-cell NHL such as diffuse large cell lymphoma (DLCL) (e.g., diffuse large B-cell lymphoma), follicular lymphoma, chronic lymphocytic leukemia/small lymphocytic lymphoma (CLL/SLL), mantle cell lymphoma (MCL), marginal zone B-cell lymphomas (e.g., mucosa-associated lymphoid tissue (MALT) lymphomas, nodal marginal zone B-cell lymphoma, splenic marginal zone B-cell lymphoma), primary mediastinal B-cell lymphoma, Burkitt lymphoma, lymphoplasmacytic lymphoma (i.e., Waldenström's macroglobulinemia), hairy cell leukemia (HCL), immunoblastic large cell lymphoma, precursor B-lymphoblastic lymphoma and primary central nervous system (CNS) lymphoma; and T-cell NHL such as precursor T-lymphoblastic lymphoma/leukemia, peripheral T-cell lymphoma (PTCL) (e.g., cutaneous T-cell lymphoma (CTCL) (e.g., mycosis fungoides, Sezary syndrome), angioimmunoblastic T-cell lymphoma, extranodal natural killer T-cell lymphoma, enteropathy type T-cell lymphoma, subcutaneous panniculitis-like T-cell lymphoma, and anaplastic large cell lymphoma); a mixture of one or more leukemia/lymphoma as described above; and multiple myeloma (MM)), heavy chain disease (e.g., alpha chain disease, gamma chain disease, mu chain disease); hemangioblastoma; hypopharynx cancer; inflammatory myofibroblastic tumors; immunocytic amyloidosis; kidney cancer (e.g., nephroblastoma a.k.a. Wilms' tumor, renal cell carcinoma); liver cancer (e.g., hepatocellular cancer (HCC), malignant hepatoma); lung cancer (e.g., bronchogenic carcinoma, small cell lung cancer (SCLC), non-small cell lung cancer (NSCLC), adenocarcinoma of the lung); leiomyosarcoma (LMS); mastocytosis (e.g., systemic mastocytosis); muscle cancer; myelodysplastic syndrome (MDS); mesothelioma; myeloproliferative disorder (MPD) (e.g., polycythemia vera (PV), essential thrombocytosis (ET), agnogenic myeloid metaplasia (AMM) a.k.a. myelofibrosis (MF), chronic idiopathic myelofibrosis, chronic myelocytic leukemia (CML), chronic neutrophilic leukemia (CNL), hypereosinophilic syndrome (HES)); neuroblastoma; neurofibroma (e.g., neurofibromatosis (NF) type 1 or type 2, schwannomatosis); neuroendocrine cancer (e.g., gastroenteropancreatic neuroendoctrine tumor (GEP-NET), carcinoid tumor); osteosarcoma (e.g., bone cancer); ovarian cancer (e.g., cystadenocarcinoma, ovarian embryonal carcinoma, ovarian adenocarcinoma); papillary adenocarcinoma; pancreatic cancer (e.g., pancreatic andenocarcinoma, intraductal papillary mucinous neoplasm (IPMN), Islet cell tumors); penile cancer (e.g., Paget's disease of the penis and scrotum); pinealoma; primitive neuroectodermal tumor (PNT); plasma cell neoplasia; paraneoplastic syndromes; intraepithelial neoplasms; prostate cancer (e.g., prostate adenocarcinoma); rectal cancer; rhabdomyosarcoma; salivary gland cancer; skin cancer (e.g., squamous cell carcinoma (SCC), keratoacanthoma (KA), melanoma, basal cell carcinoma (BCC)); small bowel cancer (e.g., appendix cancer); soft tissue sarcoma (e.g., malignant fibrous histiocytoma (MFH), liposarcoma, malignant peripheral nerve sheath tumor (MPNST), chondrosarcoma, fibrosarcoma, myxosarcoma); sebaceous gland carcinoma; small intestine cancer; sweat gland carcinoma; synovioma; testicular cancer (e.g., seminoma, testicular embryonal carcinoma); thyroid cancer (e.g., papillary carcinoma of the thyroid, papillary thyroid carcinoma (PTC), medullary thyroid cancer); urethral cancer; vaginal cancer; and vulvar cancer (e.g., Paget's disease of the vulva).

The term “enzyme” refers to macromolecular biological catalyst. Enzymes accelerate the rate of chemical reactions. The molecules upon which enzymes may act are called substrates, and the enzyme converts the substrates into different molecule known as products. Almost all metabolic processes in a cell need enzymatic catalysis in order to occur at rates fast enough to sustain life. Metabolic pathways depend upon enzymes to catalyze individual steps. Enzymes are known to catalyze more than 5000 biochemical reaction types. Most enzymes are protein. Like all catalysts, enzymes increase the reaction rate by lowering activation energy. Some enzymes can make their conversion of substrate to product occur many millions of times faster. Enzymes are like any catalyst and are not consumed in the chemical reaction nor do they alter the equilibrium of a reaction. Enzymes differ from most other catalysts by being much more specific. Enzyme activity can be affected by other molecules: inhibitors are molecules that decrease enzyme activity, and activators are molecules that increase activity. Recombinant enzymes are the result of the expression of recombinant DNA.

The terms “polynucleotide”, “nucleotide sequence”, “nucleic acid”, “nucleic acid sequence”, and “oligonucleotide” refer to a series of nucleotide bases (also called “nucleotides”) in DNA and RNA, and mean any chain of two or more nucleotides. The polynucleotides can be chimeric mixtures or derivatives or modified versions thereof, single-stranded or double-stranded. The oligonucleotide can be modified at the base moiety, sugar moiety, or phosphate backbone, for example, to improve stability of the molecule, its hybridization parameters, etc. The antisense oligonucleotide may comprise a modified base moiety which is selected from the group including, but not limited to, 5-fluorouracil, 5-bromouracil, 5-chlorouracil, 5-iodouracil, hypoxanthine, xanthine, 4-acetylcytosine, 5-(carboxyhydroxylmethyl) uracil, 5-carboxymethylaminomethyl-2-thiouridine, 5-carboxymethylaminomethyluracil, dihydrouracil, beta-D-galactosylqueosine, inosine, N6-isopentenyladenine, 1-methylguanine, 1-methylinosine, 2,2-dimethylguanine, 2-methyladenine, 2-methylguanine, 3-methylcytosine, 5-methylcytosine, N6-adenine, 7-methylguanine, 5-methylaminomethyluracil, 5-methoxyaminomethyl-2-thiouracil, beta-D-mannosylqueosine, 5′-methoxycarboxymethyluracil, 5-methoxyuracil, 2-methylthio-N6-isopentenyladenine, wybutoxosine, pseudouracil, queosine, 2-thiocytosine, 5-methyl-2-thiouracil, 2-thiouracil, 4-thiouracil, 5-methyluracil, uracil-5-oxyacetic acid methylester, uracil-5-oxyacetic acid, 5-methyl-2-thiouracil, 3-(3-amino-3-N-2-carboxypropyl) uracil, a thio-guanine, and 2,6-diaminopurine. A nucleotide sequence typically carries genetic information, including the information used by cellular machinery to make proteins and enzymes. These terms include double- or single-stranded genomic and cDNA, RNA, any synthetic and genetically manipulated polynucleotide, and both sense and antisense polynucleotides. This includes single- and double-stranded molecules, i.e., DNA-DNA, DNA-RNA and RNA-RNA hybrids, as well as “protein nucleic acids” (PNAs) formed by conjugating bases to an amino acid backbone. This also includes nucleic acids containing carbohydrate or lipids.

Polynucleotides described herein may be synthesized by standard methods known in the art, e.g., by use of an automated DNA synthesizer (such as those that are commercially available from Biosearch, Applied Biosystems, etc.). As examples, phosphorothioate oligonucleotides may be synthesized by the method of Stein et al., Nucl. Acids Res., 16, 3209, (1988), methylphosphonate oligonucleotides can be prepared by use of controlled pore glass polymer supports (Sarin et al., Proc. Natl. Acad. Sci. U.S.A. 85, 7448-7451, (1988)). Polynucleotides, such as DNA (e.g., complementary DNA (cDNA), can be created naturally or artificially. Some methods for the synthesis of DNA, well known by one skilled in the art, include: DNA replication, polymerase chain reaction, and gene synthesis. DNA replication refers to DNA biosynthesis (in vivo DNA amplification). Polymerase chain reaction refers to enzymatic DNA synthesis (in vitro DNA amplification). Gene synthesis refers to physically creating artificial gene sequences. re. In certain embodiments, cDNA is synthesized from a single stranded RNA (e.g., messenger RNA (mRNA) or microRNA) template in a reaction catalyzed by the enzyme reverse transcriptase. cDNA is often used to clone eukaryotic genes in prokaryotes. When scientists want to express a specific protein in a cell that does not normally express that protein (i.e., heterologous expression), they will transfer the cDNA that codes for the protein to the recipient cell. cDNA is also produced naturally by retroviruses (such as HIV-1, HIV-2, simian immunodeficiency virus, etc.) and then integrated into the host's genome, where it creates a provirus. cDNA is derived from eukaryotic mRNA, so it contains only exons, with no introns. cDNA is most often synthesized from mature (fully spliced) mRNA using the enzyme reverse transcriptase. This enzyme, which naturally occurs in retroviruses, operates on a single strand of mRNA, generating its complementary DNA based on the pairing of RNA base pairs (A, U, G, and C) to their DNA complements (T, A, C, and G, respectively). To obtain eukaryotic cDNA whose introns have been removed:
1) A eukaryotic cell transcribes the DNA (from genes) into RNA (pre-mRNA).
2) The same cell processes the pre-mRNA strands by removing introns, and adding a poly-A tail and 5′ methyl-guanine cap (this is known as post-transcriptional modification).
3) This mixture of mature mRNA strands is extracted from the cell. The poly-A tail of the post-transcriptional mRNA can be taken advantage of with oligo(dT) beads in an affinity chromatography assay.
4) A poly-T oligonucleotide primer is hybridized onto the poly-A tail of the mature mRNA template, or random hexamer primers can be added which contain every possible 6 base single strand of DNA and can therefore hybridize anywhere on the RNA (Reverse transcriptase requires this double-stranded segment as a primer to start its operation.)
5) Reverse transcriptase is added, along with deoxynucleotide triphosphates (A, T, G, C). This synthesizes one complementary strand of DNA hybridized to the original mRNA strand.
6) To synthesize an additional DNA strand, traditionally one would digest the RNA of the hybrid strand, using an enzyme like RNase H, or through alkali digestion method.
7) After digestion of the RNA, a single stranded DNA (ssDNA) is left and because single stranded nucleic acids are hydrophobic, it tends to loop around itself. It is likely that the ssDNA forms a hairpin loop at the 3′ end.
8) From the hairpin loop, a DNA polymerase can then use it as a primer to transcribe a complementary sequence for the ss cDNA to provide a double stranded cDNA with identical sequence as the mRNA of interest. cDNA sequences may be incorporated into a wide variety of vectors. Such vectors can be constructed by recombinant DNA technology methods standard in the art. Vectors can be plasmid, viral, or others known in the art, used for replication and expression in non-human cells or organisms, such as bacteria, yeast, or plants. Any type of plasmid, cosmid, yeast artificial chromosome, or viral vector can be used to prepare the recombinant DNA construct that can be introduced directly into the cell.

The polynucleotides may be flanked by natural regulatory (expression control) sequences or may be associated with heterologous sequences, including promoters, internal ribosome entry sites (IRES) and other ribosome binding site sequences, enhancers, response elements, suppressors, signal sequences, polyadenylation sequences, introns, 5′- and 3′-non-coding regions, and the like. The nucleic acids may also be modified by many means known in the art. Non-limiting examples of such modifications include methylation, “caps”, substitution of one or more of the naturally occurring nucleotides with an analog, and internucleotide modifications, such as, for example, those with uncharged linkages (e.g., methyl phosphonates, phosphotriesters, phosphoroamidates, carbamates, etc.) and with charged linkages (e.g., phosphorothioates, phosphorodithioates, etc.). Polynucleotides may contain one or more additional covalently linked moieties, such as, for example, proteins (e.g., nucleases, toxins, antibodies, signal peptides, poly-L-lysine, etc.), intercalators (e.g., acridine, psoralen, etc.), chelators (e.g., metals, radioactive metals, iron, oxidative metals, etc.), and alkylators. The polynucleotides may be derivatized by formation of a methyl or ethyl phosphotriester or an alkyl phosphoramidate linkage. Furthermore, the polynucleotides herein may also be modified with a label capable of providing a detectable signal, either directly or indirectly. Exemplary labels include radioisotopes, fluorescent molecules, isotopes (e.g., radioactive isotopes), biotin, and the like.

A “recombinant nucleic acid molecule” is a nucleic acid that has been modified, i.e., a non-naturally occurring nucleic acid or a genetically engineered nucleic acid. Furthermore, the term “recombinant DNA” refers to a nucleic acid sequence which is not naturally occurring or has been made by the artificial combination of two otherwise separated segments of nucleic acid sequence, i.e., by ligating together pieces of DNA that are not normally contiguous. By “recombinantly produced” is meant artificial combination often accomplished by either chemical synthesis means, or by the artificial manipulation of isolated segments of nucleic acids, e.g., by genetic engineering techniques using restriction enzymes, ligases, and similar recombinant techniques as described by, for example, Sambrook et al., Molecular Cloning, second edition, Cold Spring Harbor Laboratory, Plainview, N.Y.; (1989), or Ausubel et al., Current Protocols in Molecular Biology, Current Protocols (1989), and DNA Cloning: A Practical Approach, Volumes I and II (ed. D. N. Glover) IREL Press, Oxford, (1985); each of which is incorporated herein by reference.

The term “RNA transcript” refers to the product resulting from RNA polymerase-catalyzed transcription of a DNA sequence. When the RNA transcript is a complementary copy of the DNA sequence, it is referred to as the primary transcript, or it may be an RNA sequence derived from post-transcriptional processing of the primary transcript and is referred to as the mature RNA. “Messenger RNA (mRNA)” refers to the RNA that is without introns and can be translated into polypeptides by the cell. “cRNA” refers to complementary RNA, transcribed from a recombinant cDNA template. “cDNA” refers to DNA that is complementary to and derived from an mRNA template. The cDNA can be single-stranded or converted to double-stranded form using, for example, the Klenow fragment of DNA polymerase I.

The term “fusion protein” refers to protein created through the joining of two or more genes that originally encoded separate proteins. Translation of this fusion gene results in a single or multiple polypeptides with functional properties derived from each of the original proteins. Recombinant fusion proteins are created artificially by recombinant DNA technology for use in research, bioengineering, or as therapeutics.

The term “cell” refers to a basic structural, functional, and biological unit of all known living organisms. A cell is the smallest unit of life that can replicate independently, and cells are called the “building blocks of life”. Cells consist of cytoplasm enclosed within a membrane, which contains many biomolecules such as proteins and nucleic acids. Organisms can be classified as unicellular (consisting of a single cell; including bacteria) or multicellular (including plants and animals). While the number of cells in plants and animals varies from species to species. Most plant and animal cells are visible only under a microscope, with dimension between 1 and 100 micrometers.

The term “cell line” refers a population of cells descended from a single cell and containing the same genetic makeup. Some cell lines are designated immortalized cell lines, which are a population of cells from a multicellular organism which would normally not proliferate indefinitely but, due to mutation, have evaded normal cellular senescence (the phenomenon by which normal ploid cells cease to divide) and instead can keep undergoing division.

A “vector” may be any of a number of nucleic acid molecules or viruses, or portions thereof, that are capable of mediating entry of, e.g., transferring, transporting, etc., a nucleic acid of interest between different genetic environments or into a cell. The nucleic acid of interest may be linked to, e.g., inserted into, the vector using, e.g., restriction and ligation. Vectors include, for example, DNA or RNA plasmids, cosmids, naturally occurring or modified viral genomes or portions thereof, nucleic acids that can be packaged into viral capsids, mini-chromosomes, artificial chromosomes, etc. Plasmid vectors typically include an origin of replication (e.g., for replication in prokaryotic cells). A plasmid may include part or all of a viral genome (e.g., a viral promoter, enhancer, processing or packaging signals, and/or sequences sufficient to give rise to a nucleic acid that can be integrated into the host cell genome and/or to give rise to infectious virus). Viruses or portions thereof that can be used to introduce nucleic acids into cells may be referred to as viral vectors. Viral vectors include, e.g., adenoviruses, adeno-associated viruses, retroviruses (e.g., lentiviruses), vaccinia virus and other poxviruses, herpesviruses (e.g., herpes simplex virus), and others. Viral vectors may or may not contain sufficient viral genetic information for production of infectious virus when introduced into host cells, i.e., viral vectors may be replication-competent or replication-defective. In some embodiments, e.g., where sufficient information for production of infectious virus is lacking, it may be supplied by a host cell or by another vector introduced into the cell, e.g., if production of virus is desired. In some embodiments such information is not supplied, e.g., if production of virus is not desired. A nucleic acid to be transferred may be incorporated into a naturally occurring or modified viral genome or a portion thereof or may be present within a viral capsid as a separate nucleic acid molecule. A vector may contain one or more nucleic acids encoding a marker suitable for identifying and/or selecting cells that have taken up the vector. Markers include, for example, various proteins that increase or decrease either resistance or sensitivity to antibiotics or other agents (e.g., a protein that confers resistance to an antibiotic such as puromycin, hygromycin or blasticidin), enzymes whose activities are detectable by assays known in the art (e.g., β-galactosidase or alkaline phosphatase), and proteins or RNAs that detectably affect the phenotype of cells that express them (e.g., fluorescent proteins). Vectors often include one or more appropriately positioned sites for restriction enzymes, which may be used to facilitate insertion into the vector of a nucleic acid, e.g., a nucleic acid to be expressed. An expression vector is a vector into which a desired nucleic acid has been inserted or may be inserted such that it is operably linked to regulatory elements (also termed “regulatory sequences”, “expression control elements”, or “expression control sequences”) and may be expressed as an RNA transcript (e.g., an mRNA that can be translated into protein). Expression vectors include regulatory sequence(s), e.g., expression control sequences, sufficient to direct transcription of an operably linked nucleic acid under at least some conditions; other elements required or helpful for expression may be supplied by, e.g., the host cell or by an in vitro expression system. Such regulatory sequences typically include a promoter and may include enhancer sequences or upstream activator sequences. In some embodiments a vector may include sequences that encode a 5′ untranslated region and/or a 3′ untranslated region, which may comprise a cleavage and/or polyadenylation signal. In general, regulatory elements may be contained in a vector prior to insertion of a nucleic acid whose expression is desired or may be contained in an inserted nucleic acid or may be inserted into a vector following insertion of a nucleic acid whose expression is desired. As used herein, a nucleic acid and regulatory element(s) are said to be “operably linked” when they are covalently linked so as to place the expression or transcription of the nucleic acid under the influence or control of the regulatory element(s). For example, a promoter region would be operably linked to a nucleic acid if the promoter region were capable of effecting transcription of that nucleic acid. One of ordinary skill in the art will be aware that the precise nature of the regulatory sequences useful for gene expression may vary between species or cell types, but may in general include, as appropriate, sequences involved with the initiation of transcription, RNA processing, or initiation of translation. The choice and design of an appropriate vector and regulatory element(s) is within the ability and discretion of one of ordinary skill in the art. For example, one of skill in the art will select an appropriate promoter (or other expression control sequences) for expression in a desired species (e.g., a mammalian species) or cell type. A vector may contain a promoter capable of directing expression in mammalian cells, such as a suitable viral promoter, e.g., from a cytomegalovirus (CMV), retrovirus, simian virus (e.g., SV40), papilloma virus, herpes virus or other virus that infects mammalian cells, or a mammalian promoter from, e.g., a gene such as EF1alpha, ubiquitin (e.g., ubiquitin B or C), globin, actin, phosphoglycerate kinase (PGK), etc., or a composite promoter such as a CAG promoter (combination of the CMV early enhancer element and chicken beta-actin promoter). In some embodiments a human promoter may be used. In some embodiments, a promoter that ordinarily directs transcription by a eukaryotic RNA polymerase II (a “pol II promoter”) or a functional variant thereof is used. In some embodiments, a promoter that ordinarily directs transcription by a eukaryotic RNA polymerase I promoter, e.g., a promoter for transcription of ribosomal RNA (other than 5S rRNA) or a functional variant thereof is used. In some embodiments, a promoter that ordinarily directs transcription by a eukaryotic RNA polymerase III (a “pol III promoter”), e.g., (a U6, H1, 7SK or tRNA promoter or a functional variant thereof) may be used. One of ordinary skill in the art will select an appropriate promoter for directing transcription of a sequence of interest. Examples of expression vectors that may be used in mammalian cells include, e.g., the pcDNA vector series, pSV2 vector series, pCMV vector series, pRSV vector series, pEF1 vector series, Gateway® vectors, etc. Examples of virus vectors that may be used in mammalian cells include, e.g., adenoviruses, adeno-associated viruses, poxviruses such as vaccinia viruses and attenuated poxviruses, retroviruses (e.g., lentiviruses), Semliki Forest virus, Sindbis virus, etc. In some embodiments, regulatable (e.g., inducible or repressible) expression control element(s), e.g., a regulatable promoter, is/are used so that expression can be regulated, e.g., turned on or increased or turned off or decreased. For example, the tetracycline-regulatable gene expression system (Gossen & Bujard, Proc. Natl. Acad. Sci. 89:5547-5551, 1992) or variants thereof (see, e.g., Allen, N, et al. (2000) Mouse Genetics and Transgenics: 259-263; Urlinger, S, et al. (2000). Proc. Natl. Acad. Sci. U.S.A. 97 (14): 7963-8; Zhou, X., et al. (2006). Gene Ther. 13 (19): 1382-1390 for examples) can be employed to provide inducible or repressible expression. Other inducible/repressible systems may be used in various embodiments. For example, expression control elements that can be regulated by small molecules such as artificial or naturally occurring hormone receptor ligands (e.g., steroid receptor ligands such as naturally occurring or synthetic estrogen receptor or glucocorticoid receptor ligands), tetracycline or analogs thereof, metal-regulated systems (e.g., metallothionein promoter) may be used in certain embodiments. In some embodiments, tissue-specific or cell type specific regulatory element(s) may be used, e.g., in order to direct expression in one or more selected tissues or cell types. In some embodiments a vector capable of being stably maintained and inherited as an episome in mammalian cells (e.g., an Epstein-Barr virus-based episomal vector) may be used. In some embodiments a vector may comprise a polynucleotide sequence that encodes a polypeptide, wherein the polynucleotide sequence is positioned in frame with a nucleic acid inserted into the vector so that an N- or C-terminal fusion is created. In some embodiments the polypeptide encoded by the polynucleotide sequence may be a targeting peptide. A targeting peptide may comprise a signal sequence (which directs secretion of a protein) or a sequence that directs the expressed protein to a specific organelle or location in the cell such as the nucleus or mitochondria. In some embodiments the polypeptide comprises a tag. A tag may be useful to facilitate detection and/or purification of a protein that contains it. Examples of tags include polyhistidine-tag (e.g., 6×-His tag), glutathione-S-transferase, maltose binding protein, NUS tag, SNUT tag, Strep tag, epitope tags such as V5, HA, Myc, or FLAG. In some embodiments a protease cleavage site is located in the region between the protein encoded by the inserted nucleic acid and the polypeptide, allowing the polypeptide to be removed by exposure to the protease.

A “protein,” “peptide,” or “polypeptide” comprises a polymer of amino acid residues linked together by peptide bonds. The term refers to proteins, polypeptides, and peptides of any size, structure, or function. Typically, a protein will be at least three amino acids long. A protein may refer to an individual protein or a collection of proteins. Inventive proteins preferably contain only natural amino acids, although non-natural amino acids (i.e., compounds that do not occur in nature but that can be incorporated into a polypeptide chain) and/or amino acid analogs as are known in the art may alternatively be employed. Also, one or more of the amino acids in a protein may be modified, for example, by the addition of a chemical entity such as a carbohydrate group, a hydroxyl group, a phosphate group, a farnesyl group, an isofarnesyl group, a fatty acid group, a linker for conjugation or functionalization, or other modification. A protein may also be a single molecule or may be a multi-molecular complex. A protein may be a fragment of a naturally occurring protein or peptide. A protein may be naturally occurring, recombinant, synthetic, or any combination of these.

The terms “agroinfiltration,” “agrobacterium-infiltration,” or “agrobacterium-mediated infiltration” refer to a method used in plant biology and plant biotechnology to induce transient expression of genes in a plant, or isolated leaves from a plant, or even in cultures of plant cells, in order to produce a desired protein. In the method a suspension of Agrobacterium tumefaciens is introduced into a plant leaf by direct injection or by vacuum infiltration, or brought into association with plant cells immobilized on a porous support (plant cell packs), whereafter the bacteria transfer the desired gene into the plant cells via transfer of T-DNA. The main benefit of agroinfiltration when compared to more traditional plant transformation is speed and convenience, although yields of the recombinant protein are generally also higher and more consistent. The first step is to introduce a gene of interest to a strain of Agrobacterium tumefaciens. Subsequently, the strain is grown in a liquid culture and the resulting bacteria are washed and suspended into a suitable buffer solution. For injection, this solution is then placed in a syringe (without a needle). The tip of the syringe is pressed against the underside of a leaf while simultaneously applying gentle counterpressure to the other side of the leaf. The Agrobacterium suspension is then injected into the airspaces inside the leaf through stomata, or sometimes through a tiny incision made to the underside of the leaf. Vacuum infiltration is another way to introduce Agrobacterium deep into plant tissue. In this procedure, leaf disks, leaves, or whole plants are submerged in a beaker containing the solution, and the beaker is placed in a vacuum chamber. The vacuum is then applied, forcing air out of the intercellular spaces within the leaves via the stomata. When the vacuum is released, the pressure difference forces the Agrobacterium suspension into the leaves through the stomata into the mesophyll tissue. This can result in nearly all of the cells in any given leaf being in contact with the bacteria. Once inside the leaf that Agrobacterium remains in the intercellular space and transfers the gene of interest as part of the Ti plasmid-derived T-DNA in high copy numbers into the plants cells. The gene is then transiently expressed through RNA synthesis from appropriate species can be processed using this method, but the most common ones are Nicotiana benthamiana and less often, Nicotiana tabacum. Transient expression in cultured plant cell packs is a more recent procedure. For this technique, suspension cultured cells of tobacco (e.g., NT1 or BY2 cell lines of Nicotiana tabacum) are immobilized by filtration onto a porous support to form a well-aerated cell pack, then incubated with recombinant Agrobacterium for a time to allow T-DNA transfer, before refiltration to remove excess bacteria and liquid. Incubation of the cell pack in a humid environment for time periods up to several days allows transient expression of protein. Secreted proteins can be washed out of the cell pack by application of buffer and further filtration.

The terms “assessing”, “determining”, “evaluating”, and “assaying” are used interchangeably herein to refer to any form of detection or measurement, and include determining whether a substance, signal, enzymatic activity, disease, condition, etc., is present or not. The result of an assessment may be expressed in qualitative and/or quantitative terms. Assessing may be relative or absolute. “Assessing the presence of” includes determining the amount of something that is present or determining whether it is present or absent.

A “biological process” may be any set of operations or molecular events, with a defined beginning and end, pertinent to the functioning of integrated living units, e.g., cells, tissues, organs, and organisms. Typically it is a series of events accomplished by one or more ordered assemblies of molecular functions. A “biological pathway” may be any series of actions and/or interactions by and among molecules in a cell that leads to a certain product or a change in a cell. Typically a biological process encompasses or is carried out via one or more biological pathways. Biological pathways include, for example, pathways pertaining to metabolism, genetic information processing (e.g., transcription, translation, RNA transport, RNA degradation; protein folding, sorting, degradation, post-translational modification; DNA replication and repair), environmental information processing (e.g., membrane transport, signal transduction), and cellular processes (e.g., cell cycle, endocytosis, vesicle trafficking), etc. It will be appreciated that the various afore-mentioned biological processes encompass multiple specific pathways. In some embodiments a biological pathway or process is conserved in that the pathway or process is recognizably present in both yeast and mammalian cells.

### BRIEF DESCRIPTION OF THE DRAWINGS

The accompanying drawings, which constitute a part of this specification, illustrate several exemplary embodiments of the invention and together with the description, serve to explain certain principles of the invention. The embodiments disclosed in the drawings are exemplary and do not limit the scope of this disclosure.

FIG. 1 shows the chemical structures of twenty known kavalactones.

FIG. 2 shows the chemical structures of three known flavokavains.

FIG. 3 shows the biosynthetic pathway of kavalactones and flavokavains.

FIG. 4 shows the in vitro enzymatic production of bisnoryangonin (kavalactone intermediate with 6-styryl-4-hydroxy-2-pyrone backbone) and naringenin chalcone (flavokavain intermediate with chalcone backbone) using purified recombinant PmSPS1, PmSPS2, and PmCHS enzymes from p-coumaric acid. CTAL (p-coumaroyltriacetic acid lactone is a known in vitro derailment byproduct of chalcone synthase (CHS), which is not produced in vivo.

FIG. 5 shows the enzymatic production of compounds with a 6-styryl-4-hydroxy-2-pyrone backbone from carboxylic acid compounds with single bond or double bond at the 7,8-position: p-coumaric acid, phloretic acid, and hydrocinnamic acid.

FIG. 6 shows the in vitro enzymatic production starting from phloretic acid and hydrocinnamic acid of compounds with a 6-styryl-4-hydroxy-2-pyrone backbone and single bond at the 7,8-position.

FIG. 7 shows the positions of hydroxyl groups on the 6-styryl-4-hydroxy-2-pyrone backbone that can be methylated by PmOMT4 and PmOMT1.

FIG. 8 shows liquid chromatography-mass spectrometry (LC-MS) results (m/z at retention time) of final products of enzymatic processes using different substrates and a combination of PmCL1, PmSPS1, and two methyltransferases PmOMT4 and PmOMT1.

FIG. 9 shows the results of an in vitro enzyme assay demonstrating the activity of PmRDCT10 to reduce the C₅-C₆ double bond in kavalactones. While the combination of Pm4CL1, PmSPS1, and PmOMT4 is sufficient to produce the kavalactone desmethoxyyangonin from cinnamic acid, PmRDCT10 is required to produce kavain, which carries a single bond at the C₅-C₆ position. The identity of desmethoxyyangonin and kavain was confirmed with pure standards, including their retention times and utilizing tandem mass spectrometry (MS/MS) as shown in the bottom panel.

FIG. 10 shows the pathway to produce methylenedioxy bridge-containing kavalactones such as 5,6-dehydromethysticin starting from caffeic acid.

FIG. 11 shows the LC-MS traces of mass 273.075 m/z corresponding to [C₁₅H₁₂O₅+H]⁺ ion of 5,6-dehydromethysticin in agrobacterium-infiltrated N. benthamiana leaves. Each leaf was infiltrated with a mixture of agrobacterial strains carrying plasmids with the indicated enzymes.

FIG. 12 shows the production of bisnoryangonin in vivo in E. coli. The E. coli BW27784 strain carrying expression plasmids with the indicated enzymes was incubated for 24 hours in the presence of 1 mM p-coumaric acid.

FIG. 13 shows the production of bisnoryangonin and naringenin chalcone in vivo in the baker's yeast S. cerevisiae. The yeast strain BY4743 carrying expression plasmids with the indicated enzymes was incubated for 2 days in the presence of 2 mM p-coumaric acid.

FIG. 14 shows the production of the kavalactone yangonin in vivo in the plant Nicotiana benthamiana through agrobacterium-mediated infiltration. This assay utilized the native Nicotiana 4CL enzyme.

### DETAILED DESCRIPTION OF CERTAIN EMBODIMENTS

The present disclosure provides methods, compositions, proteins, nucleic acids, cells, vectors, compounds, reagents, and systems for the production of kavalactones, flavokavains, and intermediates thereto. Described herein are the biosynthetic pathways and enzymes useful for the conversion of cinnamic acid derivatives and phenylpropanoic acid derivatives to kavalactones and flavokavains in a series of in vivo and/or in vitro enzymatic reactions. The enzymatic synthesis of kavalactones utilizes 4-coumarate-CoA ligase Pm4CL1 (or an enzyme that is at least 80% identical to SEQ ID NO: 1), styrylpyrone synthase PmSPS1 (or an enzyme that is at least 80% identical to SEQ ID NO: 2) or styrylpyrone synthase PmSPS2 (or an enzyme that is at least 80% identical to SEQ ID NO: 3), methyltransferase PmOMT4 (or an enzyme that is at least 80% identical to SEQ ID NO: 5), and any number of methyltranferases (e.g., PmOMT1 (or an enzyme at least 80% identical to SEQ ID NO: 6)), cytochrome P450 enzymes (e.g., PmMDB1 (or an enzyme at least 80% identical to SEQ ID NO: 7)), and/or NADPH-dependent reductases (e.g., PmRDCT10 (or an enzyme at least 80% identical to SEQ ID NO: 8)). The enzymatic synthesis of flavokavains utilizes at least one 4-coumarate-CoA ligase Pm4CL1 (or an enzyme that is at least 80% identical to SEQ ID NO: 1), chalcone synthase PmCHS (or an enzyme that is at least 80% identical to SEQ ID NO: 4), methyltranferase PmOMT4 (or an enzyme that is at least 80% identical to SEQ ID NO: 5, and any number of methyltranferases (e.g., PmOMT1 (or an enzyme at least 80% identical to SEQ ID NO: 6)) and cytochrome P450 enzyme PmMDB1 (or an enzyme at least 80% identical to SEQ ID NO: 7). Any of the methods to produce compounds described herein can optionally utilize chemical means or a combination of reactions utilizing enzymes described herein and chemical means.

Any of the methods described herein may include culturing cells or cultivating plants expressing enzymes described herein and isolating one or more compounds described herein from such cells or plants. Methods described herein can include harvesting tissue (e.g., leaves, roots) of a plant expressing enzymes described herein and processing the harvested tissue to isolate one or more compounds described herein therefrom. Compounds may be isolated using solvent extraction, chromatography, and/or other separation methods known in the art.

Any of the enzymatic individual steps may be combined, omitted, or done through other means and still be within the scope of the invention.

Enzymes and cDNA

Sequence identity is the amount of characters which match exactly between two different sequences. Hereby, gaps are not counted and the measurement is relational to the shorter of the two sequences. This has the effect that sequence identity is not transitive, i.e. if sequence A=B and B=C then A is not necessarily equal C (in terms of the identity distance measure): A: AAGGCTT; B: AAGGC; C: AAGGCAT. Here identity(A,B)=100% (5 identical nucleotides/min(length(A),length(B))). Identity(B,C)=100%, but identity(A,C)=85% ((6 identical nucleotides/7)). So 100% identity does not mean two sequences are the same. Sequence identity can be applied to polypeptides and polynucleotide. For example, the phrase an enzyme “that is at least Y % identical to SEQ ID NO: X”, can be understood to apply the description above for comparing amino acid sequences to determine that an enzyme is at least 80% identical to a enzyme with a SEQ ID NO described herein.

In certain embodiments, the enzyme (polypeptide) or DNA (polynucleotide) is a variant of a natural or artificial enzyme or DNA. A “variant” of a particular polypeptide or polynucleotide has one or more additions, substitutions, and/or deletions with respect to the polypeptide or polynucleotide, which may be referred to as the “original polypeptide” or “original polynucleotide”, respectively. An addition may be an insertion or may be at either terminus. A variant may be shorter or longer than the original polypeptide or polynucleotide. The term “variant” encompasses “fragments”. A “fragment” is a continuous portion of a polypeptide or polynucleotide that is shorter than the original polypeptide or polynucleotide. In some embodiments a variant comprises or consists of a fragment. In some embodiments a fragment or variant is at least 20%, 30%, 40%, 50%, 60%, 70%, 80%, 90%, 95%, 96%, 97%, 98%, 99%, or more as long as the original polypeptide or polynucleotide. A fragment may be an N-terminal, C-terminal, or internal fragment. In some embodiments a variant polypeptide comprises or consists of at least one domain of an original polypeptide. In some embodiments a variant polypeptide or polynucleotide comprises or consists of a polypeptide or polynucleotide that is at least 70%, 80%, 90%, 95%, 96%, 97%, 98%, 99%, or more identical in sequence to the original polypeptide or polynucleotide over at least 50%, 60%, 70%, 80%, 90%, 95%, 96%, 97%, 98%, 99%, or 100% of the original polypeptide or polynucleotide. In some embodiments the sequence of a variant polypeptide comprises or consists of a sequence that has N amino acid differences with respect to an original sequence, wherein N is any integer up to 1%, 2%, 5%, or 10% of the number of amino acids in the original polypeptide, where an “amino acid difference” refers to a substitution, insertion, or deletion of an amino acid. In some embodiments a substitution is a conservative substitution. Conservative substitutions may be made, e.g., on the basis of similarity in side chain size, polarity, charge, solubility, hydrophobicity, hydrophilicity and/or the amphipathic nature of the residues involved. In some embodiments, conservative substitutions may be made according to Table A, wherein amino acids in the same block in the second column and in the same line in the third column may be substituted for one another other in a conservative substitution. Certain conservative substitutions are substituting an amino acid in one row of the third column corresponding to a block in the second column with an amino acid from another row of the third column within the same block in the second column.

TABLE A

| | | |
|---|---|---|
| Aliphatic | Non-polar | G A P |
| | | I L V |
| | Polar - uncharged | C S T M |
| | | N Q |
| | Polar - charged | D E |
| | | K R |
| Aromatic | | H F W Y |

In some embodiments, proline (P), cysteine (C), or both are each considered to be in an individual group. Within a particular group, certain substitutions may be of particular interest in certain embodiments, e.g., replacements of leucine by isoleucine (or vice versa), serine by threonine (or vice versa), or alanine by glycine (or vice versa).

In some embodiments a variant is a biologically active variant, i.e., the variant at least in part retains at least one activity of the original polypeptide or polynucleotide. In some embodiments a variant at least in part retains more than one or substantially all known biologically significant activities of the original polypeptide or polynucleotide. An activity may be, e.g., a catalytic activity, binding activity, ability to perform or participate in a biological structure or process, etc. In some embodiments an activity of a variant may be at least 10%, 20%, 30%, 40%, 50%, 60%, 70%, 80%, 90%, 95%, or more, of the activity of the original polypeptide or polynucleotide, up to approximately 100%, approximately 125%, or approximately 150% of the activity of the original polypeptide or polynucleotide, in various embodiments. In some embodiments a variant, e.g., a biologically active variant, comprises or consists of a polypeptide at least 95%, 96%, 97%, 98%, 99%, 99.5% or 100% identical to an original polypeptide over at least 70%, 75%, 80%, 85%, 90%, 95%, 96%, 97%, 98%, or 99% or 100% of the original polypeptide. In some embodiments an alteration, e.g., a substitution or deletion, e.g., in a functional variant, does not alter or delete an amino acid or nucleotide that is known or predicted to be important for an activity, e.g., a known or predicted catalytic residue or residue involved in binding a substrate or cofactor. Variants may be tested in one or more suitable assays to assess activity.

Each amino acid in the enzyme sequence can be encoded by multiple DNA codons. Therefore, there are many possible cDNA sequences that will translated to the same amino acid sequences described herein and produce the same enzyme. Examples of cDNA sequences that encode the enzymes described herein are shown in Example 6.

In certain embodiments, an enzyme of the present disclosure is at least 80%, 85%, 90%, 95%, or 100% identical to an enzyme with a SEQ ID NO described herein. In certain embodiments, an enzyme of the present disclosure is greater than 95%, 96%, 97%, 98%, or 99% identical to an enzyme with a SEQ ID NO described herein.

The enzymes described herein (e.g., 4-coumarate-CoA ligase Pm4CL1 or an enzyme that is at least 80% identical to SEQ ID NO: 1, styrylpyrone synthase PmSPS1 or an enzyme that is at least 80% identical to SEQ ID NO: 2, styrylpyrone synthase PmSPS2 or an enzyme that is at least 80% identical to SEQ ID NO: 3, chalcone synthase PmCHS or an enzyme that is at least 80% identical to SEQ ID NO: 4, methyltransferase PmOMT4 or an enzyme that is at least 80% identical to SEQ ID NO: 5, methyltransferase PmOMT1 or an enzyme that is at least 80% identical to SEQ ID NO: 6, cytochrome P450 enzyme PmMDB1 or an enzyme that is at least 80% identical to SEQ ID NO: 7, and NADPH-dependent reductase PmRDCT10 or an enzyme that is at least 80% identical to SEQ ID NO: 8) are isolated or derived from wildtype, mutant or recombinant Piper methysticum. In certain embodiments, the enzymes are produced using recombinant technology. In certain embodiments, an acid-thiol ligase (EC 6.2.1.A) is used to form carbon-sulfur bonds, wherein A is an integer between 1 and 100, inclusive. The acid-thiol ligase is isolated or derived from wildtype, mutant, or recombinant Piper methysticum. The acid-thiol ligase can be used as an unpurified enzyme, partially purified enzyme, or purified enzyme. In certain embodiments, the amino acid sequence of the enzyme is at least 80% identical to SEQ ID NO: 1, designated 4-coumarate-CoA ligase PmCL1, wherein SEQ ID NO: 1 is MKMVVDTIATDRCVYRSKLPDIEIKNDMSLHNYCFQNIGAYRDNPCLINGSTGEVYTYG EVETTARRVAAGLHRMGVQQREVIMILLPNSPEFVFAFLGASFRGAMSTTANPFYTPQEI AKQVKASGAKLIVTMSAYVDKVRDLAEERGVKVVCVDAPPPGCSHFSELSGADESELP EVDIDPDDVVALPYSSGTTGLPKGVMLTHRSQVTSVAQQVDGENPNLYFRPDDVLLCV LPLFHIYSLNSVLFCGLRVGAAILIMQKFEITALMELVQKYKVTIAPIVPPIVLAIAKSPLV DKYDLSSIRTVMSGAAPMGKELEDAVRAKLPNAKLGQGYGMTEAGPVLSMCLAFAKE PFEIKSGSCGTVVRNAQLKIVDPETGAYLPRNQPGEICIRGSQIMKGYLNDAAATQRTID KEGWLHTGDIGYVDDDEELFIVDRLKEIIKYKGFQVAPAELEAILITHPNIADAAVVPMK DEAAGEVPVAFVVTSNGSVISEDEIKQFISKQVVFYKRINRVFFVDSIPKAPSGKILRKDL RGRLAAGIPK. In certain embodiments, the acid-thiol ligase is 4-coumarate-CoA ligase (EC 6.2.1.12).

In certain embodiments, a transferase or synthase (EC 2.3.1.B) is used to form styrylpyrones from coenzyme A esters of cinnamic acids, wherein B is an integer between 1 and 300, inclusive. In certain embodiments, a transferase or synthase (EC 2.3.1.B) is used to form styrylpyrones from coenzyme A esters of cinnamic acids, wherein B is an integer between 1 and 300, inclusive. The transferase or synthase is isolated or derived from wildtype, mutant, or recombinant Piper methysticum. The transferase or synthase can be used as an unpurified enzyme, partially purified enzyme, or purified enzyme. In certain embodiments, the synthase belongs to an enzyme family of type III polyketide synthases. In certain embodiments, the amino acid sequence of the enzyme is at least 80% identical to SEQ ID NO: 2, designated styrylpyrone synthase PmSPS1, wherein SEQ ID NO: 2 is MSKTVEDRAAQRAKGPATVLAIGTATPANV VYQTDYPDYYFRVTKSEHMTKLKNKFQRMCDRSTIKKRYMVLTEELLEKNLSLCTYME PSLDARQDILVPEVPKLGKEAADEAIAEWGRPKSEITHLIFCTTCGVDMPGADYQLTKLL GLRSSVRRTMLYQQGCFGGGTVLRLAKDLAENNAGARVLVVCSEITTAVNFRGPSDTH LDLLVGLALFGDGAAAVIVGADPDPTLERPLFQIVSGAQTILPDSEGAINGHLREVGLTIR LLKDVPGLVSMNIEKCLMEAFAPMGIHDWNSIFWIAHPGGPTILDQVEAKLGLKEEKLK STRAVLREYGNMSSACVLFILDEVRKRSMEEGKTTTGEGFDWGVLFGFGPGFTVETVVL HSMPIPKADEGR. In certain embodiments, the amino acid sequence of the enzyme is at least 80% identical to SEQ ID NO: 3, designated styrylpyrone synthase PmSPS2, wherein SEQ ID NO: 3 is MSKMVEEHWAAQRARGPATVLAIGTANPPNVLYQADYPDFYFRVTKSEHMT QLKEKFKRICDKSAIRKRHLHLTEELLEKNPNICAHMAPSLDARQDIAVVEVPKLAKEA ATKAIKEWGRPKSDITHLIFCTTCGVDMPGADYQLTTLLGLRPTVRRTMLYQQGCFAGG TVLRHAKDFAENNRGARVLAVCSEFTVMNFSGPSEAHLDSMVGMALFGDGASAVIVG ADPDFAIERPLFQLVSTTQTIVPDSDGAIKCHLKEVGLTLHLVKNVPDLISNNMDKILEEA FAPLGIRDWNSIFWTAHPGGAAILDQLEAKLGLNKEKLKTTRTVLREYGNMSSACVCFV LDEMRRSSLEEGKTTSGEGLEWGILLGFGPGLTVETVVLRSVPISTAN. In certain embodiments, the amino acid sequence of the enzyme is at least 80% identical to SEQ ID NO: 4, designated chalcone synthase PmCHS, wherein SEQ ID NO: 4 is MSKTVEEIWAAQRARGPA TVLAIGTAAPANVVYQADYPDYYFRITKSEHMTELKEKFRRMCDKSMITKRHMHLSEE LLKNNPDICAYMAPSLDARQDMVVVEVPKLGKEAAAKAIKEWGRPKSAITHLIFCTTSG VDMPGADFQLTKLLGLCPSVRRTMLYQQGCFAGGTVLRLAKDLAENNAGARVLVVCS EITAVTFRGPSETHLDSMVGQALFGDGASAIIVGADPDPVIERPLFQIVSAAQTILPDSDG AIDGHLREVGLTFHLLKDVPGLISKNIEKSLKEAFAPLGIDDWNSIFWIVHPGGPAILDQV EAKLRLKVEKLKTTRTVLSEYGNMSSACVLFILDEMRRNSMEEGKATTGEGLHWGVLF GFGPGLTVETVVLHSLPIAEAN. In certain embodiments, the synthase is chalcone synthase (EC 2.3.1.74).

The amino acid sequences of the polyketide synthases described herein contain conserved catalytic triads. In certain embodiments, the conserved catalytic triad of PmSPS1 is Cys164, His304, and Asn337. In certain embodiments, the conserved catalytic triad of PmSPS2 is Cys164, His303, and Asn336. In certain embodiments, the conserved catalytic triad of PmCHS is Cys164, His303, and Asn336.

In certain embodiments, an O-methyltransferase (EC 2.1.1.C) is used to methylate hydroxyl groups substituting styrylpyrones, wherein C is an integer between 1 and 200, inclusive. In certain embodiments, an O-methyltransferase (EC 2.1.1.C) is used to methylate hydroxyl groups substituting chalcones, wherein C is an integer between 1 and 200, inclusive. The O-methyltransferase is isolated or derived from wildtype, mutant, or recombinant Piper methysticum. The O-methyltransferase can be used as an unpurified enzyme, partially purified enzyme, or purified enzyme. In certain embodiments, the amino acid sequence of the enzyme is at least 80% identical to SEQ ID NO: 5, designated O-methyltransferase PmOMT4, wherein SEQ ID NO: 5 is MEQAVFKDQSPSRDDIDEELFQSALYLSTAVVTVPAAIMAANDLDVLQ IIAKAGPGAHLSPTEIVSHLPTRNPNAAAALHRILRVLASHSILECSSRCEGEAKYGLRPV CKFFLNDKDGVSLNAMPSFVQSRVFIDSWQYMKDAVLEGVVPFEKAYGMPFYQFQAV NTKFKETFAKAMAAHSTLVVKKMLDTYNGFEGLTELMDVAGGTGSTLNLIVSKYPQIK GTNFDLKHVIEAAPNYPGVKHLSGDMFDSIPSAKNIIMKWILHNWSDEHCVKLLKNCYT SLPEFGKLIVVDSIVGEDVDAGLTTTNVFGCDFTMLTFFPNAKERTREEFQDLAKASGFS TFKPICCAYGVWVMEFHK. In certain embodiments, the amino acid sequence of the enzyme is at least 80% identical to SEQ ID NO: 6, designated O-methyltransferase PmOMT1, wherein SEQ ID NO: 6 is MNDQELHGYSQNAQPQLWNLLLSFINSMSLKCAVELGIPDIIHSHAQ TPINITDLAASIPIPPNKTSQFRRLMRLLVHSNVFSVHKREDGDEGFLLTPMSRILVTSND NNGGNLSPFVSMMVDPSLVSPWHFLGQWLKGNDTQGTPFRMCHGEEMWDWANKYP DFNKKFNMAMVCDSQYLMKIIVKKCATAFEGKRSLIDVGGGTGGAARSIAEAFPDIQEV SVLDLPHVVAGLPNDSRVKFVGGDMFHTIPPADVVLLKAIFHGWNDEECIKILKNCKKA IPSKEEGGKVMILDMVVNSAPGDHMITEDQYFMDLMMITYARGLERDENEWKKLFKD AGFTSYKITHGLGTSSLIELYP. In certain embodiments, the synthase is chalcone synthase (EC 2.3.1.74).

In certain embodiments, a methylenedioxy bridge-forming enzyme is used to form a methylenedioxy moiety from a hydroxyl group and a methoxy group each separately bonded to adjacent carbons of an aromatic ring belonging to a styrylpyrone or chalcone compound. The methylenedioxy bridge-forming enzyme is isolated or derived from wildtype, mutant, or recombinant Piper methysticum. The methylenedioxy bridge-forming enzyme can be used as an unpurified enzyme, partially purified enzyme, or purified enzyme. In certain embodiments, methylenedioxy bridge-forming enzyme belongs to the P450 enzyme family. In certain embodiments, methylenedioxy bridge-forming enzyme belongs to the CYP719 enzyme family. In certain embodiments, the amino acid sequence of the enzyme is at least 80% identical to SEQ ID NO: 7, designated methylenedioxy bridge-forming enzyme PmMDB1, wherein SEQ ID NO: 7 is MEQAQWVDPTLLPAFVGIIFFFLGMFFGRSSLGAGKGAAPRSTSSTEWPDGPPKLPII GNLHQLNKGGELVHHNLAKLAQSYDRAMTIWVGSWGPMIVVSDADLAWEVLVTKSP DFAGRVLSKLSHLFNANYNTVVAYDAGPQWQSLRRGLQHGPLGPAHVSAQARFHEED MKLLVSDMMRAAQKGGSNGVVEPLAYVRRATIRFLSRLCFGEAFNDEAFVEGMDEAV EETIGATGHARILDAFYFTRHLPIIRRSFIDTVNAKKKIESLVRPLLSRPAPPGSYLHFLLST DAPENMIIFRIFEVYLLGVDSTASTTTWALAFLVSNQQAQEKLHNELAQYCASQNNQIIK ADDVGKLSYLLGVVKETMRMKPIAPLAVPHKTLKETMLDGKRVAAGTTVVVNLYAVH YNPKLWPEPEQFRPERFVVGASGGNGGGSSEYMLQSYLPFGGGMRSCAGMEVGKLQV AMVVANLVMAFKWLPEEEGKMPDLAEDMTFVLMMKKPLAAKIVPRA.

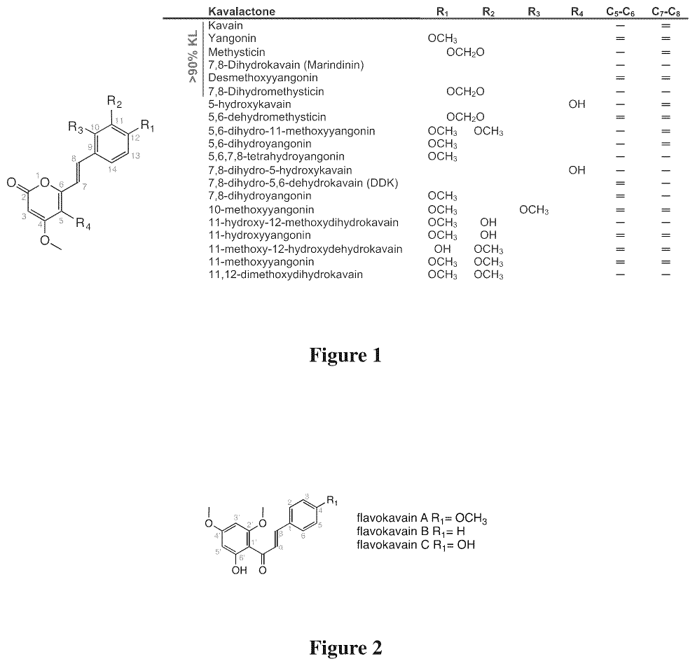

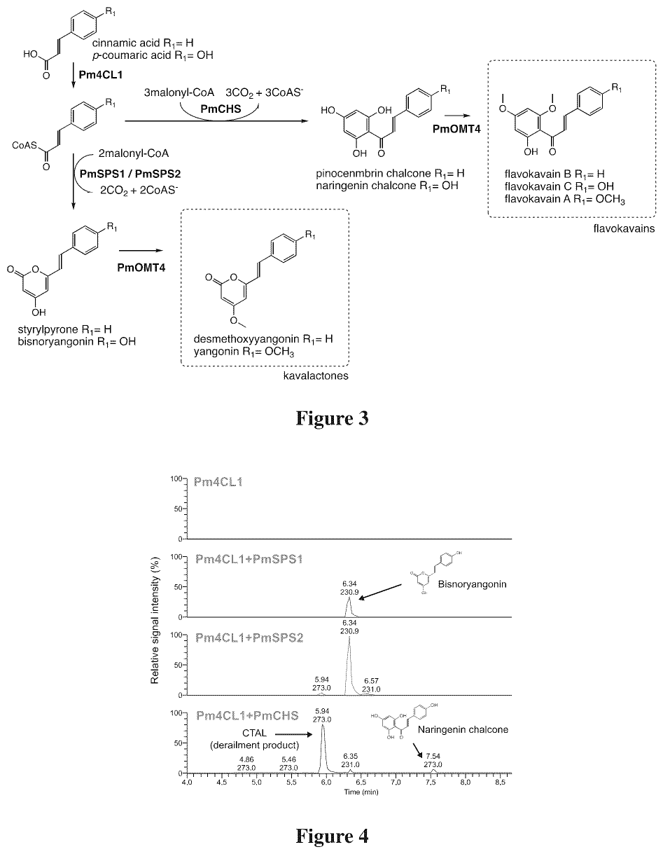

In certain embodiments, a dehydrogenase or reductase (EC 1.1.1.D) is used to reduce the C₅-C₆ double bond of kavalactones into a single bond (FIG. 1), wherein D is an integer between 1 and 450, inclusive. The dehydrogenase or reductase is isolated or derived from wildtype, mutant, or recombinant Piper methysticum. The dehydrogenase or reductase can be used as an unpurified enzyme, partially purified enzyme, or purified enzyme. In certain embodiments, the reductase is a NADPH-dependent reductase. In certain embodiments, the amino acid sequence of the enzyme is at least 80% identical to SEQ ID NO: 8, designated NADPH-dependent reductase PmRDCT10, wherein SEQ ID NO: 8 is METERKSRICVTGAGG FVASWVVKLFLSKGYLVHGTVRDLGEEKTAHLRKLEGAYHNLQLFKADLLDYESLLGA ITGCDGVLHVATPVPSSKTAYSGTELVKTAVNGTLNVLRACTEAKVKKVIYVSSTAAVL VNPNLPKDKIPDEDCWTDEEYCRTTPFFLNWYCIAKTAAEKNALEYGDKEGINVISICPS YIFGPMLQPTINSSNLELLRLMKGDDESIENKFLLMVDVRDVAEAILLLYEKQETSGRYIS SPHGMRQSNLVEKLESLQPGYNYHKNFVDIKPSWTMISSEKLKKLGWKPRPLEDTISET VLCFEEHGLLENE.

Protein sequencing encompasses the process of determining the amino acid sequence of all or part of a protein or peptide. The two major methods of protein sequencing are Edman degradation using a protein sequenator and mass spectrometry. Typically, only part of the protein's sequence needs to be determined experimentally in order to identify the protein with reference to databases of protein sequences deduced from the DNA sequences of their genes.

Prior to attempting to find the ordered sequence of a protein, it is often desirable to know the unordered amino acid composition of a protein as this knowledge can be used to facilitate the discovery of errors in the sequencing process or to distinguish between ambiguous results. Knowledge of the frequency of certain amino acids may also be used to choose which protease to use for digestion of the protein. The misincorporation of low levels of non-standard amino acids (e.g. norleucine) into proteins may also be determined. A generalized method often referred to as amino acid analysis for determining amino acid frequency is as follows: 1) hydrolyze a known quantity of protein into its constituent amino acids; and 2) separate and quantify the amino acids.

Hydrolysis is done by heating a sample of the protein in 6 M hydrochloric acid to approximately 100 to 110° C. for 24 hours or longer. Proteins with many bulky hydrophobic groups may require longer heating periods. However, these conditions are so vigorous that some amino acids (e.g., serine, threonine, tyrosine, tryptophan, glutamine, and cysteine) are degraded. To circumvent this problem the following strategy is employed: 1) heat separate samples for different times; 2) analyze the composition of each resulting solution; and 3) extrapolating back to zero hydrolysis time. A variety of reagents are known to prevent or reduce degradation, such as thiol reagents or phenol to protect tryptophan and tyrosine from attack by chlorine, and pre-oxidizing cysteine. In addition, measuring the quantity of ammonia evolved allows for the determination of the extent of amide hydrolysis.

After the hydrolysis step, the amino acids can be separated by ion-exchange chromatography and then derivatized to facilitate their detection. More commonly, the amino acids are derivatized then resolved by reversed phase HPLC.

The first major method for protein sequencing is the Edman degradation, which consists of the following steps: 1) break any disulfide bridges in the protein with a reducing agent like 2-mercaptoethanol. A protecting group such as iodoacetic acid may be necessary to prevent the bonds from re-forming; 2) separate and purify the individual chains of the protein complex, if there are more than one; 3) determine the amino acid composition of each chain; 4) determine the terminal amino acids of each chain; 5) break each chain into fragments under 50 amino acids long; 6) separate and purify the fragments; 7) determine the sequence of each fragment; 8) repeat with a different pattern of cleavage; and 9) construct the sequence of the overall protein.

Peptides longer than about 50-70 amino acids long cannot be sequenced reliably by the Edman degradation. Therefore, long protein chains need to be broken up into small fragments that can then be sequenced individually. Digestion is done either by endopeptidases such as trypsin or pepsin or by chemical reagents such as cyanogen bromide. Different enzymes give different cleavage patterns, and the overlap between fragments can be used to construct an overall sequence.

The seventh step of the Edman degradation begins with adsorbing the peptide to be sequenced onto a solid surface. One common substrate is glass fiber coated with polybrene, a cationic polymer. The Edman reagent, phenylisothiocyanate (PITC), is added to the adsorbed peptide, together with a mildly basic buffer solution of trimethylamine. This reacts with the amine group of the N-terminal amino acid. The terminal amino acid can then be selectively detached by the addition of anhydrous acid. The derivative then isomerizes to give a substituted phenylthiohydantoin, which can be washed off and identified by chromatography, and the cycle can be repeated. The efficiency of each step is about 98%, which allows about 50 amino acids to be reliably determined.

Automated Edman sequencers called protein sequenators are now in widespread use, and are able to sequence peptides up to approximately 50 amino acids long. A sample of the protein or peptide is immobilized in the reaction vessel of the protein sequenator and the Edman degradation is performed. Each cycle releases and derivatizes one amino acid from the protein or peptide's N-terminus and the released amino acid derivative is then identified by HPLC. The sequencing process is done repetitively for the whole polypeptide until the entire measurable sequence is established or for a pre-determined number of cycles.

The second major method for protein sequencing is mass spectrometry, which consists of the following steps:
1) The protein is isolated, typically by SDS-PAGE or chromatography.
2) The isolated protein may be chemically modified to stabilize cysteine residues (e.g., S-amidomethylation or S-carboxymethylation).
3) The protein is digested with a specific protease to generate peptides. Trypsin, which cleaves selectively on the C-terminal side of lysine or arginine residues, is the most commonly used protease. Its advantages include i) the frequency of Lys and Arg residues in proteins, ii) the high specificity of the enzyme, iii) the stability of the enzyme and iv) the suitability of tryptic peptides for mass spectrometry.
4) The peptides may be desalted to remove ionizable contaminants and subjected to MALDI-TOF mass spectrometry. Direct measurement of the masses of the peptides may provide sufficient information to identify the protein, but further fragmentation of the peptides inside the mass spectrometer is often used to gain information about the peptides' sequences. Alternatively, peptides may be desalted and separated by reversed phase HPLC and introduced into a mass spectrometer via an ESI source. LC-ESI-MS may provide more information than MALDI-MS for protein identification but typically requires more instrument time.
5) Depending on the type of mass spectrometer, fragmentation of peptide ions may occur via a variety of mechanisms such as collision-induced dissociation (CID) or post-source decay (PSD). In each case, the pattern of fragment ions of a peptide provides information about its sequence.
6) Information including the measured mass of the putative peptide ions and those of their fragment ions is then matched against calculated mass values from the conceptual (in silico) proteolysis and fragmentation of databases of protein sequences. A successful match will be found if its score exceeds a threshold based on the analysis parameters. Even if the actual protein is not represented in the database, error-tolerant matching allows for the putative identification of a protein based on similarity to homologous proteins. A variety of software packages are available to perform this analysis.
7) Software packages usually generate a report showing the identity (accession code) of each identified protein, its matching score, and provide a measure of the relative strength of the matching where multiple proteins are identified.
8) A diagram of the matched peptides on the sequence of the identified protein is often used to show the sequence coverage (% of the protein detected as peptides). When the protein is thought to be significantly smaller than the matched protein, the diagram may suggest whether the protein is an N- or C-terminal fragment of the matched protein.

### Production of CoA Esters of Formula (II)

In one aspect, the present disclosure provides methods for producing a compound of Formula (II) from a compound of Formula (I), or a salt thereof, and coenzyme A (CoA) using an enzyme that is at least 80% identical to Pm4CL1 (SEQ ID NO: 1), wherein:

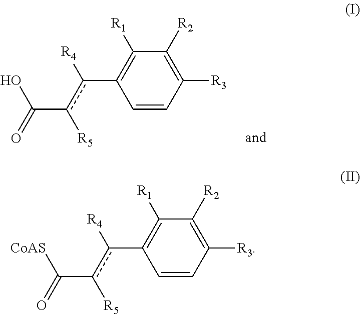

In certain embodiments, the condensation reaction of a compound of Formula (I) with coenzyme A to produce a compound of Formula (II) utilizes adenosine triphosphate. In certain embodiments, the condensation reaction of a compound of Formula (I) with coenzyme A to produce a compound of Formula (II) is performed in vitro. In certain embodiments, the in vitro condensation reaction is performed with an unpurified enzyme that is at least 80% identical to Pm4CL1 (SEQ ID NO: 1). In certain embodiments, the in vitro condensation reaction is performed with a partially purified enzyme that is at least 80% identical to Pm4CL1 (SEQ ID NO: 1). In certain embodiments, the in vitro condensation reaction is performed with an purified enzyme that is at least 80% identical to Pm4CL1 (SEQ ID NO: 1). In certain embodiments, the condensation reaction of a compound of Formula (I) with coenzyme A to produce a compound of Formula (II) is performed in vivo.

In certain embodiments, [symbol] is a single bond. In certain embodiments, [symbol] is a double bond.

In certain embodiments, each of R₁, R₂, and R₃ independently is hydrogen, optionally substituted, cyclic or acyclic aliphatic, or —ORₓ, wherein Rₓ is hydrogen or optionally substituted, cyclic or acyclic aliphatic. In certain embodiments, R₁ is hydrogen. In certain embodiments, R₂ is hydrogen. In certain embodiments, R₃ is hydrogen. In certain embodiments, R₁ is —OH. In certain embodiments, R₂ is —OH. In certain embodiments, R₃ is —OH. In certain embodiments, R₁ is —OCH₃. In certain embodiments, R₂ is —OCH₃. In certain embodiments, R₃ is —OCH₃. In certain embodiments, R₁, R₂, and R₃ are hydrogen. In certain embodiments, R₁, R₂, and R₃ are —OH. In certain embodiments, R₁ and R₃ are —OH. In certain embodiments, R₂ and R₃ are —OH. In certain embodiments, R₂ is —OCH₃.

In certain embodiments, each of R₄ and R₅ independently is hydrogen or optionally substituted, cyclic or acyclic aliphatic. In certain embodiments, both R₄ and R₅ are hydrogen.

The methods to produce a compound of Formula (II) include condensing coenzyme A (CoA) with a compound of Formula (I) selected from the group consisting of:

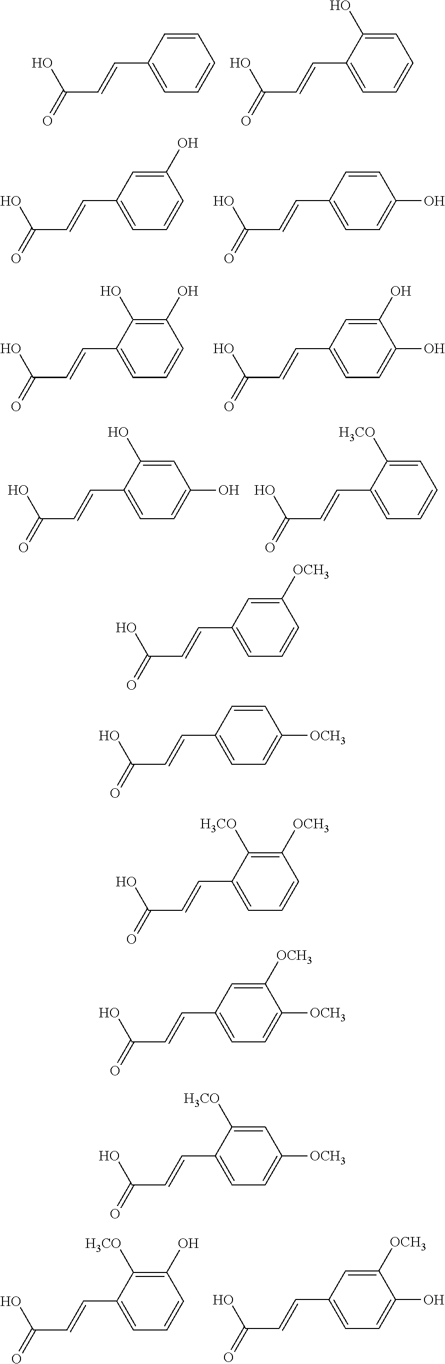

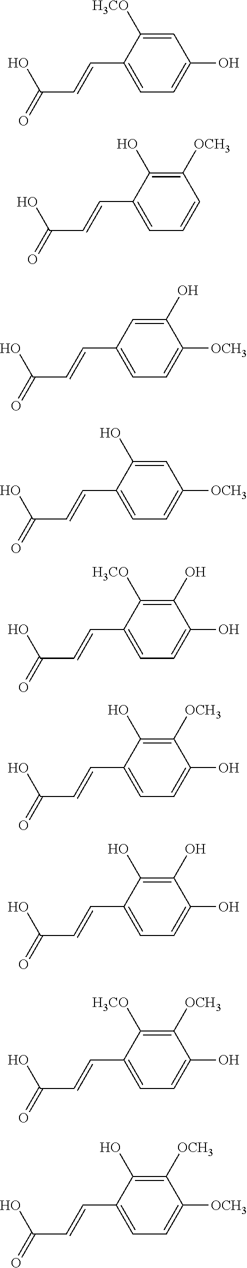

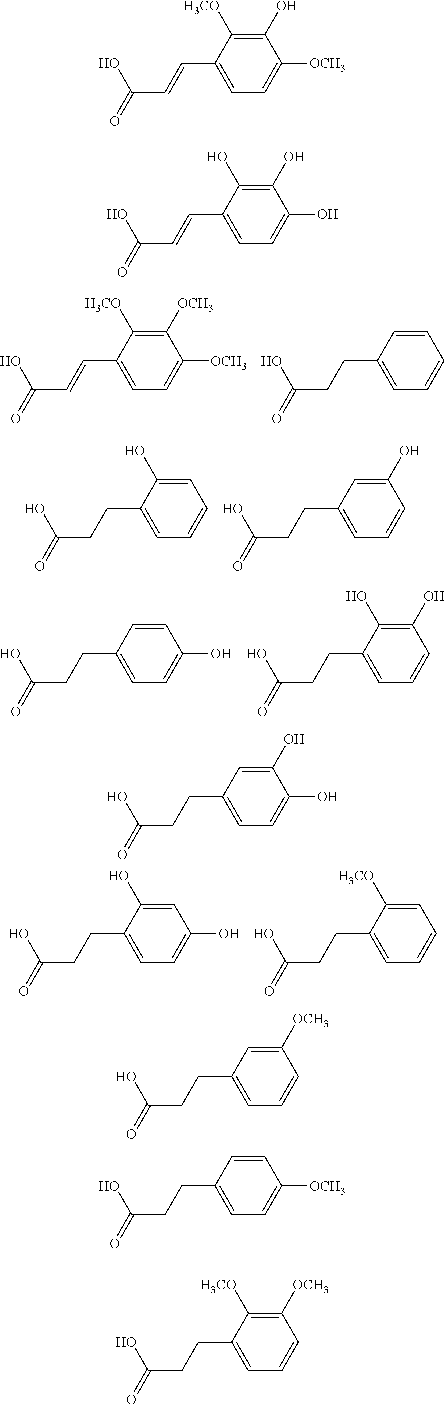

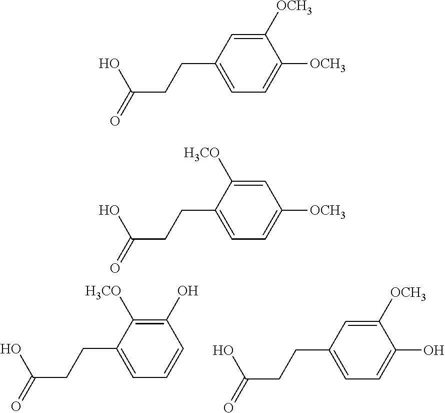

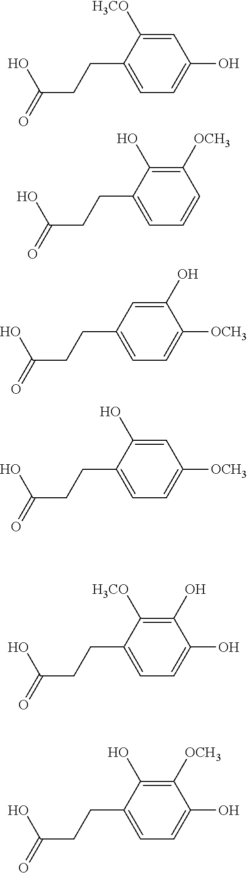 [SMILES: COc1cc(O)ccc1CCC(=O)O ; COc1cccc(CCC(=O)O)c1O ; COc1ccc(CCC(=O)O)cc1O ; COc1ccc(CCC(=O)O)c(O)c1 ; COc1c(CCC(=O)O)ccc(O)c1O ; COc1c(O)ccc(CCC(=O)O)c1O]

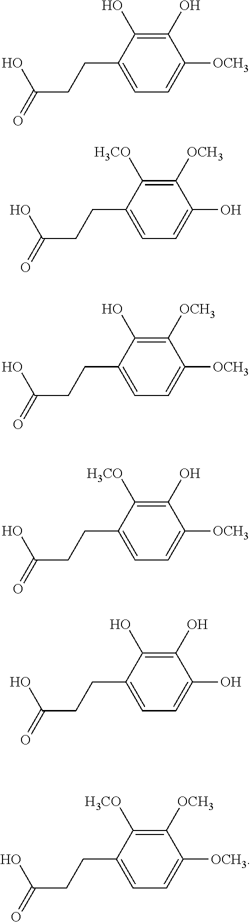 [SMILES: COc1ccc(CCC(=O)O)c(O)c1O ; COc1c(O)ccc(CCC(=O)O)c1OC ; COc1ccc(CCC(=O)O)c(O)c1OC ; COc1ccc(CCC(=O)O)c(OC)c1O ; O=C(O)CCc1ccc(O)c(O)c1O ; COc1ccc(CCC(=O)O)c(OC)c1OC]

The methods to produce a compound of Formula (II) include culturing cells engineered to express an enzyme that is at least 80% identical to Pm4CL1 (SEQ ID NO: 1). In certain embodiments, the enzyme is at least 80%, 85%, 90%, 95%, or 100% identical to Pm4CL1 (SEQ ID NO: 1). In certain embodiments, the enzyme is purified before being used in a reaction with a compound of Formula (I). In certain embodiments, the enzyme is partially purified before being used in a reaction with a compound of Formula (I).

In certain embodiments, the enzyme that is at least 80% identical to Pm4CL1 (SEQ ID NO: 1) is a component of a fusion protein. A fusion protein may be created by joining two or more gene or gene segments that code for separate proteins. Translation of this fusion gene results in a single or multiple polypeptides with functional properties derived from each of the original proteins. A polyfunctional protein is a single protein that has at least two different activities, wherein that functionality is a native biological function or the result of an engineered enzyme fusion. Thus, a fusion protein may include multiple activities such as those described herein for the kavalactone or flavokavain pathway enzymes described herein (i.e., 4-coumarate-CoA ligase Pm4CL1 (at least 80% identical to SEQ ID NO: 1), styrylpyrone synthase PmSPS1 (at least 80% identical to SEQ ID NO: 2), PmSPS2 (at least 80% identical to SEQ ID NO: 3), PmCHS (at least 80% identical to SEQ ID NO: 4), methyltransferase PmOMT4 (at least 80% identical to SEQ ID NO: 5), methyltransferase PmOMT1 (at least 80% identical to SEQ ID NO: 6), cytochrome P450 enzyme PmMDB1 (at least 80% identical to SEQ ID NO: 7), and NADPH-dependent reductase PmRDCT10 (at least 80% identical to SEQ ID NO: 8)).

The enzyme that is at least 80% identical to Pm4CL1 (SEQ ID NO: 1) is heterologous to the host cell. In certain embodiments, the enzyme that is at least 80% identical to Pm4CL1 (SEQ ID NO: 1) is recombinantly produced. In certain embodiments, the enzyme that is at least 80% identical to Pm4CL1 (SEQ ID NO: 1) is obtained from a wildtype organism. In certain embodiments, the enzyme that is at least 80% identical to Pm4CL1 (SEQ ID NO: 1) is obtained from a mutant organism. In certain embodiments, the enzyme that is at least 80% identical to Pm4CL1 (SEQ ID NO: 1) is obtained from a genetically-modified organism. In certain embodiments, the organism is a non-human organism. In certain embodiments, the non-human organism is selected from group consisting of bacteria, yeast, and plant. In certain embodiments, the organism is a plant. In certain embodiments, the plant is Piper methysticum.

A nucleic acid encoding the enzyme may be introduced into the cell via a vector (e.g., plasmids, viral vectors, cosmids, and artificial chromosomes). In certain embodiments, the nucleic acid encoding the enzyme is cDNA derived from the gene encoding the enzyme that is at least 80% identical to Pm4CL1 (SEQ ID NO: 1). In some embodiments, multiple cDNAs comprising sequences from different genes (e.g., 2, 3, 4, 5, or more genes) described herein (e.g., 4-coumarate-CoA ligase Pm4CL1 (at least 80% identity to SEQ ID NO: 1), styrylpyrone synthase PmSPS1 (at least 80% identical to SEQ ID NO: 2), PmSPS2 (at least 80% identical to SEQ ID NO: 3), PmCHS (at least 80% identical to SEQ ID NO: 4), methyltransferase PmOMT4 (at least 80% identical to SEQ ID NO: 5), methyltransferase PmOMT1 (at least 80% identical to SEQ ID NO: 6), cytochrome P450 enzyme PmMDB1 (at least 80% identical to SEQ ID NO: 7), and NADPH-dependent reductase PmRDCT10 (at least 80% identical to SEQ ID NO: 8)), are introduced into the same cell individually, or together, or as part of a single nucleic acid.

The host cells expressing the enzyme that is at least 80% identical to Pm4CL1 (SEQ ID NO: 1) may be prokaryotic cells, such as bacterial cells (e.g., Escherichia coli cells), or eukaryotic cells, such as yeast cells or plant cells. In certain embodiments, the host cell is capable of expressing two or more kavalactone or flavokavain pathway enzymes described herein. In certain embodiments, the host cell is a bacteria cell and is a wildtype, mutant, recombinant, or genetically engineered form of Escherichia coli. In certain embodiments, the host cell is a yeast cell and is a wildtype, mutant, recombinant, or genetically engineered form of Saccharomyces cerevisiae. In certain embodiments, the host cell is a plant cell and is a wildtype, mutant, recombinant, or genetically engineered form of Nicotiana benthamiana.

### Production of 6-styryl-4-hydroxyl-2-pyrone compounds of Formula (III)

Some aspects of the present disclosure provides methods for producing a compound of Formula (III) from a compound of Formula (II), or a salt thereof, and malonyl-CoA using an enzyme that is at least 80% identical to PmSPS1 (SEQ ID NO: 2). Some aspects of the present disclosure provides methods for producing a compound of Formula (III) from a compound of Formula (II), or a salt thereof, and malonyl-CoA using an enzyme that is at least 80% identical to PmSPS2 (SEQ ID NO: 3). The structure of a compound of Formula (II) and a structure of a compound of Formula (III) are as follows:

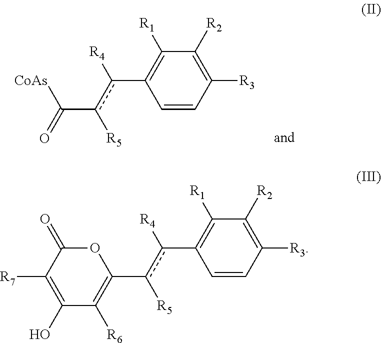

In certain embodiments, the reaction of a compound of Formula (II) with malonyl-CoA to produce a compound of Formula (III) utilizes two or more molar equivalents of malonyl-CoA relative to the compound of Formula (II). In certain embodiments, the reaction of a compound of Formula (II) with malonyl-CoA to produce a compound of Formula (III) is performed in vitro. In certain embodiments, the reaction of a compound of Formula (II) with malonyl-CoA to produce a compound of Formula (III) is performed in vivo.

In certain embodiments, [symbol] is a single bond. In certain embodiments, [symbol] is a double bond.

In certain embodiments, each of R₁, R₂, R₃, R₆, and R₇ independently is hydrogen, optionally substituted, cyclic or acyclic aliphatic, or ORₓ, wherein Rₓ is hydrogen or optionally substituted, cyclic or acyclic aliphatic. In certain embodiments, R₁ is hydrogen. In certain embodiments, R₂ is hydrogen. In certain embodiments, R₃ is hydrogen. In certain embodiments, R₁ is —OH. In certain embodiments, R₂ is —OH. In certain embodiments, R₃ is —OH. In certain embodiments, R₁ is —OCH₃. In certain embodiments, R₂ is —OCH₃. In certain embodiments, R₃ is —OCH₃. In certain embodiments, R₁, R₂, and R₃ are hydrogen. In certain embodiments, R₁, R₂, and R₃ are —OH. In certain embodiments, R₁ and R₃ are —OH. In certain embodiments, R₂ and R₃ are —OH. In certain embodiments, R₂ is —OCH₃. In certain embodiments, R₆ is hydrogen. In certain embodiments, R₆ is —OH. In certain embodiments, R₇ is hydrogen. In certain embodiments, R₆ is —OH and R₇ is hydrogen. In certain embodiments, both R₆ and R₇ are hydrogen.

In certain embodiments, each of R₄ and R₅ independently is hydrogen or optionally substituted, cyclic or acyclic aliphatic. In certain embodiments, both R₄ and R₅ are hydrogen.

The methods to produce a compound of Formula (III) include reacting malonyl-CoA with a compound of Formula (II) selected from the group consisting of:

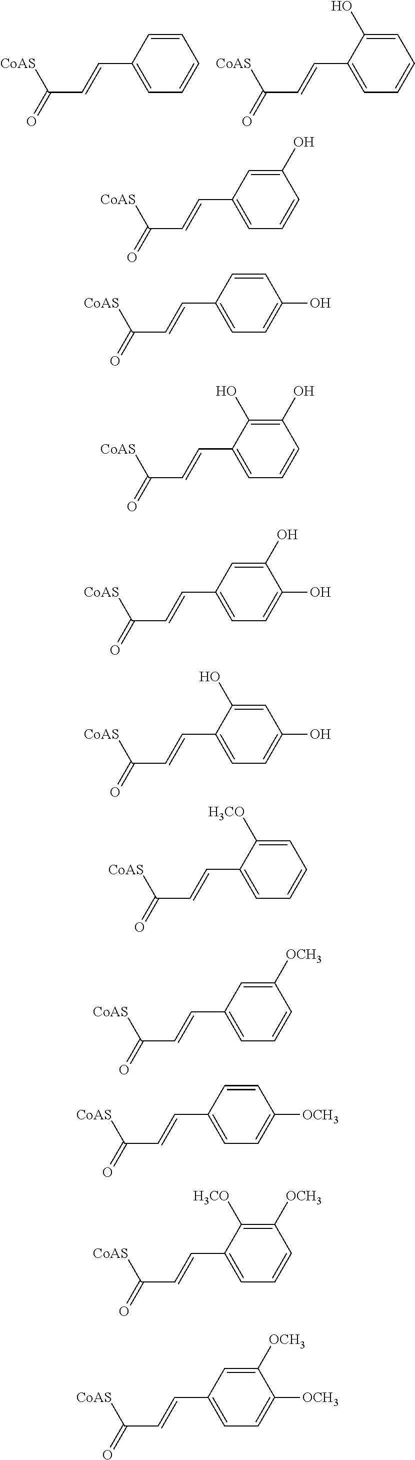

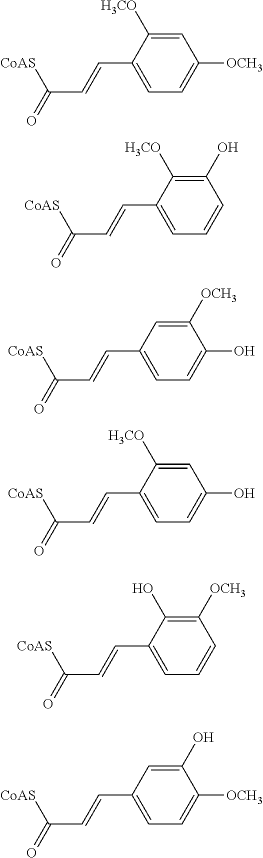

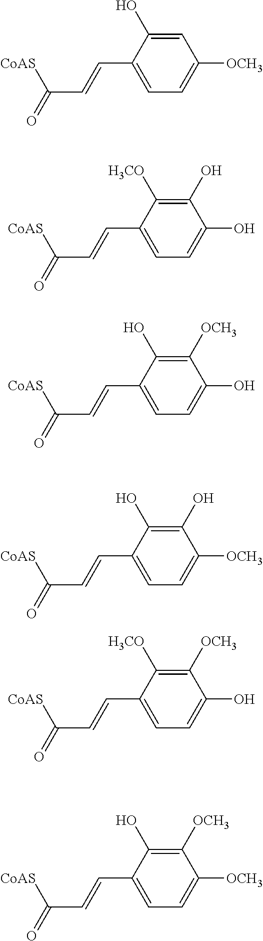

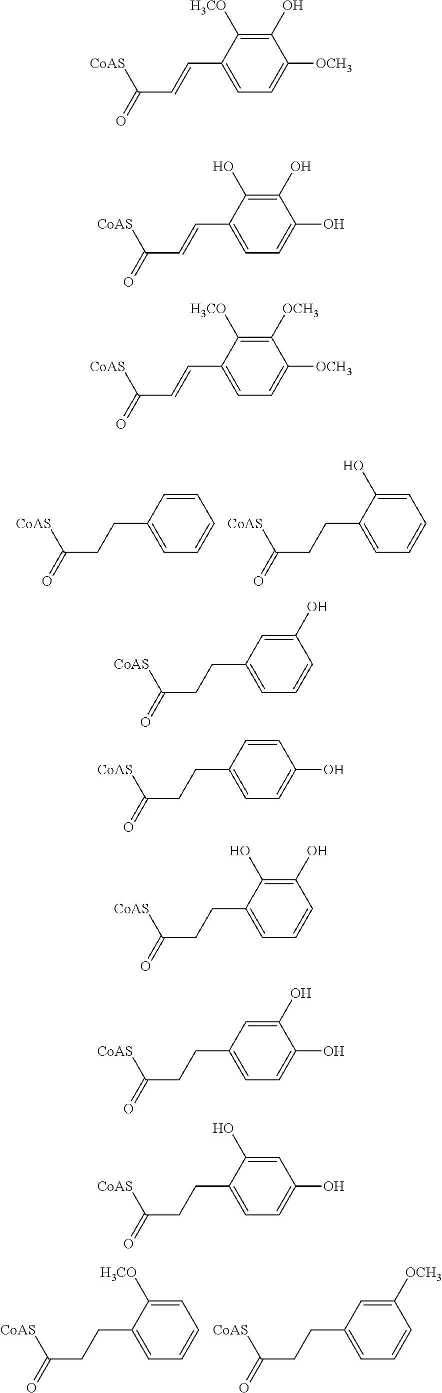

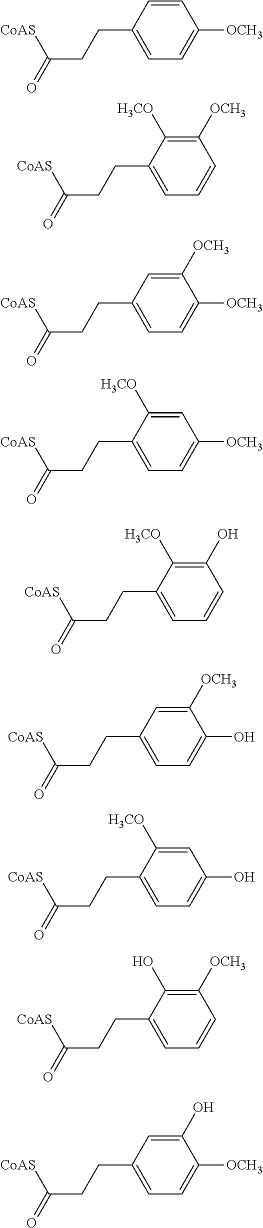

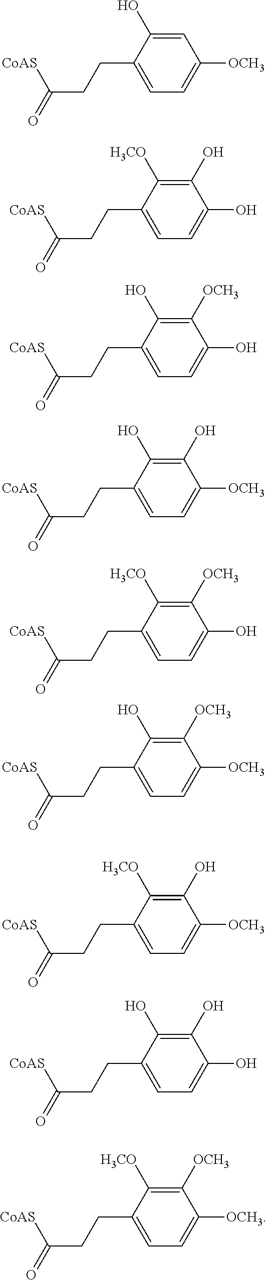

The methods to produce a compound of Formula (III) include culturing cells engineered to express an enzyme that is at least 80% identical to PmSPS1 (SEQ ID NO: 2). The methods to produce a compound of Formula (III) include culturing cells engineered to express an enzyme that is at least 80% identical to PmSPS2 (SEQ ID NO: 3). In certain embodiments, the enzyme is at least 80%, 85%, 90%, 95%, or 100% identical to PmSPS1 (SEQ ID NO: 2). In certain embodiments, the enzyme is at least 80%, 85%, 90%, 95%, or 100% identical to PmSPS2 (SEQ ID NO: 3). In certain embodiments, the enzyme is purified before reacting with a compound of Formula (II). In certain embodiments, the enzyme is partially purified before reacting with a compound of Formula (II).

In certain embodiments, the enzyme that is at least 80% identical to PmSPS1 (SEQ ID NO: 2) is a component in a fusion protein. In certain embodiments, the enzyme that is at least 80% identical to PmSPS2 (SEQ ID NO: 3) is a component in a fusion protein. A fusion protein may be created by joining two or more gene or gene segments that code for separate proteins. Translation of this fusion gene results in a single or multiple polypeptides with functional properties derived from each of the original proteins. A polyfunctional protein is a single protein that has at least two different activities, wherein that functionality is a native biological function or the result of an engineered enzyme fusion. Thus, a fusion protein may include multiple activities such as those described herein for the kavalactone or flavokavain pathway enzymes described herein (i.e., 4-coumarate-CoA ligase Pm4CL1 (at least 80% identical to SEQ ID NO: 1), styrylpyrone synthase PmSPS1 (at least 80% identical to SEQ ID NO: 2), PmSPS2 (at least 80% identical to SEQ ID NO: 3), PmCHS (at least 80% identical to SEQ ID NO: 4), methyltransferase PmOMT4 (at least 80% identical to SEQ ID NO: 5), methyltransferase PmOMT1 (at least 80% identical to SEQ ID NO: 6), cytochrome P450 enzyme PmMDB1 (at least 80% identical to SEQ ID NO: 7), and NADPH-dependent reductase PmRDCT10 (at least 80% identical to SEQ ID NO: 8)).

The enzyme that is at least 80% identical to PmSPS1 (SEQ ID NO: 2) is heterologous to the host cell. The enzyme that is at least 80% identical to PmSPS2 (SEQ ID NO: 3) is heterologous to the host cell. In certain embodiments, the enzyme that is at least 80% identical to PmSPS1 (SEQ ID NO: 2) is recombinantly produced. In certain embodiments, the enzyme that is at least 80% identical to PmSPS2 (SEQ ID NO: 3) is recombinantly produced. In certain embodiments, the enzyme that is at least 80% identical to PmSPS1 (SEQ ID NO: 2) is obtained from a wildtype organism. In certain embodiments, the enzyme that is at least 80% identical to PmSPS1 (SEQ ID NO: 2) is obtained from a mutant organism. In certain embodiments, the enzyme that is at least 80% identical to PmSPS1 (SEQ ID NO: 2) is obtained from a genetically-modified organism. In certain embodiments, the enzyme that is at least 80% identical to PmSPS2 (SEQ ID NO: 3) is obtained from a wildtype organism. In certain embodiments, the enzyme that is at least 80% identical to PmSPS2 (SEQ ID NO: 3) is obtained from a mutant organism. In certain embodiments, the enzyme that is at least 80% identical to PmSPS2 (SEQ ID NO: 3) is obtained from a genetically-modified organism. In certain embodiments, the organism is a non-human organism. In certain embodiments, the non-human organism is selected from group consisting of bacteria, yeast, and plant. In certain embodiments, the organism is a plant. In certain embodiments, the plant is Piper methysticum.

A nucleic acid encoding the enzyme may be introduced into the cell in a vector (e.g., plasmids, viral vectors, cosmids, and artificial chromosomes). In certain embodiments, the nucleic acid is cDNA derived from the amino acid sequence of the enzyme that is at least 80% identical to PmSPS1 (SEQ ID NO: 2). In certain embodiments, the nucleic acid is cDNA derived from the amino acid sequence of the enzyme that is at least 80% identical to PmSPS2 (SEQ ID NO: 3). In some embodiments multiple cDNAs comprising sequences complementary to different genes (e.g., 2, 3, 4, 5, or more genes) described herein (i.e., 4-coumarate-CoA ligase Pm4CL1 (at least 80% identical to SEQ ID NO: 1), styrylpyrone synthase PmSPS1 (at least 80% identical to SEQ ID NO: 2), PmSPS2 (at least 80% identical to SEQ ID NO: 3), PmCHS (at least 80% identical to SEQ ID NO: 4), methyltransferase PmOMT4 (at least 80% identical to SEQ ID NO: 5), methyltransferase PmOMT1 (at least 80% identical to SEQ ID NO: 6), cytochrome P450 enzyme PmMDB1 (at least 80% identical to SEQ ID NO: 7), and NADPH-dependent reductase PmRDCT10 (at least 80% identical to SEQ ID NO: 8)), are introduced into the same cell individually, or together, or as part of a single nucleic acid.

The host cells expressing the enzyme that is at least 80% identical to PmSPS1 (SEQ ID NO: 2) may be prokaryotic cells, such as bacterial cells (e.g., Escherichia coli cells), or eukaryotic cells, such as yeast cells or plant cells. The host cells expressing the enzyme that is at least 80% identical to PmSPS2 (SEQ ID NO: 3) may be prokaryotic cells, such as bacterial cells (e.g., Escherichia coli cells), or eukaryotic cells, such as yeast cells or plant cells. In certain embodiments, the host cell is capable of expressing two or more kavalactone or flavokavain pathway enzymes described herein. In certain embodiments, the host cell is a bacteria cell and is a wildtype, mutant, recombinant, or genetically engineered form of Escherichia coli. In certain embodiments, the host cell is a yeast cell and is a wildtype, mutant, recombinant, or genetically engineered form of Saccharomyces cerevisiae. In certain embodiments, the host cell is a plant cell and is a wildtype, mutant, recombinant, or genetically engineered form of Nicotiana benthamiana.

In certain embodiments, the method for producing a compound of Formula (III) utilizes a compound of Formula (I), or a salt thereof, as the starting material and comprises the steps: condensing a compound of Formula (I), or a salt thereof, with coenzyme A (CoA) using a recombinant enzyme that is at least 80% identical to Pm4CL1 (SEQ ID NO: 1) to produce a compound of Formula (II); and reacting a compound of Formula (II), or a salt thereof, with malonyl-CoA using an enzyme that is at least 80% identical to PmSPS1 (SEQ ID NO: 2) to produce a compound of Formula (III).

In certain embodiments, the method for producing a compound of Formula (III) utilizes a compound of Formula (I), or a salt thereof, as the starting material and comprises the steps: condensing a compound of Formula (I), or a salt thereof, with coenzyme A (CoA) using a recombinant enzyme that is at least 80% identical to Pm4CL1 (SEQ ID NO: 1) to produce a compound of Formula (II); and reacting a compound of Formula (II), or a salt thereof, with malonyl-CoA using an enzyme that is at least 80% identical to PmSPS2 (SEQ ID NO: 3) to produce a compound of Formula (III).

### Production of Methylated 6-styryl-4-hydroxyl-2-pyrone Compounds of Formula (IV)

Some aspects of the present disclosure provides methods for producing a compound of Formula (IV) from a compound of Formula (III), or a salt thereof, and S-adenosylmethionine using an enzyme that is at least 80% identical to PmOMT4 (SEQ ID NO: 5). Some aspects of the present disclosure provides methods for producing a compound of Formula (IV) from a compound of Formula (III), or a salt thereof, and S-adenosylmethionine using an enzyme that is at least 80% identical to PmOMT1 (SEQ ID NO: 6). The structure of a compound of Formula (III) and a structure of a compound of Formula (IV) are as follows:

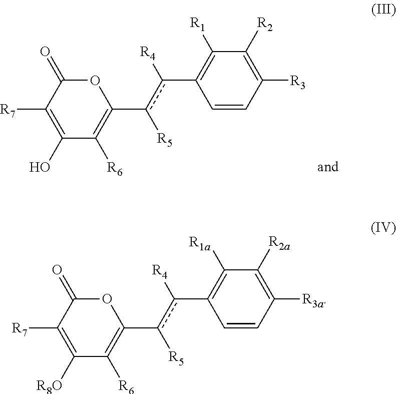

In certain embodiments, the reaction of a compound of Formula (III) with S-adenosylmethionine to produce a compound of Formula (IV) is performed in vitro. In certain, embodiments, the reaction of a compound of Formula (III) with S-adenosylmethionine to produce a compound of Formula (IV) is performed in vivo.

In certain embodiments, [symbol] is a single bond. In certain embodiments, [symbol] is a double bond.

In certain embodiments, each of R₁, R₂, R₃, R₁ₐ, R₂ₐ, R₃ₐ, R₆, and R₇ independently is hydrogen, optionally substituted, cyclic or acyclic aliphatic, or —ORₓ, wherein Rₓ is hydrogen or optionally substituted, cyclic or acyclic aliphatic. In certain embodiments, R₁ is hydrogen. In certain embodiments, R₂ is hydrogen. In certain embodiments, R₃ is hydrogen. In certain embodiments, R₁ is —OH. In certain embodiments, R₂ is OH. In certain embodiments, R₃ is —OH. In certain embodiments, R₁ is —OCH₃. In certain embodiments, R₂ is —OCH₃. In certain embodiments, R₃ is —OCH₃. In certain embodiments, R₁, R₂, and R₃ are hydrogen. In certain embodiments, R₁, R₂, and R₃ are —OH. In certain embodiments, R₁ and R₃ are —OH. In certain embodiments, R₂ and R₃ are —OH. In certain embodiments, R₂ is —OCH₃. In certain embodiments, R₆ is hydrogen. In certain embodiments, R₆ is —OH. In certain embodiments, R₇ is hydrogen. In certain embodiments, R₆ is —OH and R₇ is hydrogen. In certain embodiments, both R₆ and R₇ are hydrogen. In certain embodiments, wherein R₈ is —CH₃. In certain embodiments, R₁ₐ is hydrogen. In certain embodiments, R₂ₐ is hydrogen. In certain embodiments, R₃ₐ is hydrogen. In certain embodiments, R₁ₐ is —OH. In certain embodiments, R₂ₐ is OH. In certain embodiments, R₃ₐ is —OH. In certain embodiments, R₁ₐ is —OCH₃. In certain embodiments, R₂ₐ is —OCH₃. In certain embodiments, R₃ₐ is —OCH₃. In certain embodiments, R₁ₐ, R₂ₐ, and R₃ₐ are hydrogen. In certain embodiments, R₁ₐ, R₂ₐ, and R₃ₐ are —OCH₃. In certain embodiments, R₁ₐ and R₃ₐ are —OCH₃. In certain embodiments, R₂ₐ and R₃ₐ are —OCH₃. In certain embodiments, R₂ₐ and R₃ₐ are —OCH₃ and R₈ is —CH₃. In certain embodiments, R₂ₐ and R₃ₐ are —OH and R₈ is —H.

In certain embodiments, each of R₄ and R₅ independently is hydrogen or optionally substituted, cyclic or acyclic aliphatic. In certain embodiments, R₄ and R₅ are hydrogen.

A compound of Formula (III) can provide different compounds of Formula (IV) depending on the choice to utilize only an enzyme that is at least 80% identical to PmOMT4 (SEQ ID NO: 5), or only an enzyme that is at least 80% identical to PmOMT1 (SEQ ID NO: 6), or both enzymes (FIG. 7). In certain embodiments, R₈ is —CH₃ when a compound of Formula (III) is reacted with an enzyme that is at least 80% identical to PmOMT4 (SEQ ID NO: 5). In certain embodiments, R₈ is hydrogen when a compound of Formula (III) is reacted with an enzyme that is at least 80% identical to PmOMT1 (SEQ ID NO: 6). In certain embodiments, R₈ is —CH₃ when a compound of Formula (III) is reacted with both an enzyme that is at least 80% identical to PmOMT4 (SEQ ID NO: 5) and an enzyme that is at least 80% identical to PmOMT1 (SEQ ID NO: 6). In certain embodiments, if R₁ is —OH, then R₁ₐ is —OH when a compound of Formula (III) is reacted with an enzyme that is at least 80% identical to PmOMT4 (SEQ ID NO: 5). In certain embodiments, if R₁ is —OH, then R₁ₐ is —OCH₃ when a compound of Formula (III) is reacted with an enzyme that is at least 80% identical to PmOMT1 (SEQ ID NO: 6). In certain embodiments, if R₁ is —OH, then R₁ₐ is —OCH₃ when a compound of Formula (III) is reacted with both an enzyme that is at least 80% identical to PmOMT4 (SEQ ID NO: 5) and an enzyme that is at least 80% identical to PmOMT1 (SEQ ID NO: 6). In certain embodiments, if R₂ is —OH, then R₂ₐ is —OCH₃ when a compound of Formula (III) is reacted with an enzyme that is at least 80% identical to PmOMT4 (SEQ ID NO: 5). In certain embodiments, if R₂ is —OH, then R₂ₐ is —OH when a compound of Formula (III) is reacted with an enzyme that is at least 80% identical to PmOMT1 (SEQ ID NO: 6). In certain embodiments, if R₂ is —OH, then R₂ₐ is —OCH₃ when a compound of Formula (III) is reacted with both an enzyme that is at least 80% identical to PmOMT4 (SEQ ID NO: 5) and an enzyme that is at least 80% identical to PmOMT1 (SEQ ID NO: 6). In certain embodiments, if R₃ is —OH, then R₃ₐ is —OCH₃ when a compound of Formula (III) is reacted with an enzyme that is at least 80% identical to PmOMT4 (SEQ ID NO: 5). In certain embodiments, if R₃ is —OH, then R₃ₐ is —OH when a compound of Formula (III) is reacted with an enzyme that is at least 80% identical to PmOMT1 (SEQ ID NO: 6). In certain embodiments, if R₃ is —OH then R₃ₐ is —OCH₃ when a compound of Formula (III) is reacted with both an enzyme that is at least 80% identical to PmOMT4 (SEQ ID NO: 5) and an enzyme that is at least 80% identical to PmOMT1 (SEQ ID NO: 6).

The methods to produce a compound of Formula (IV) include reacting malonyl-CoA with a compound of Formula (III) selected from the group consisting of:

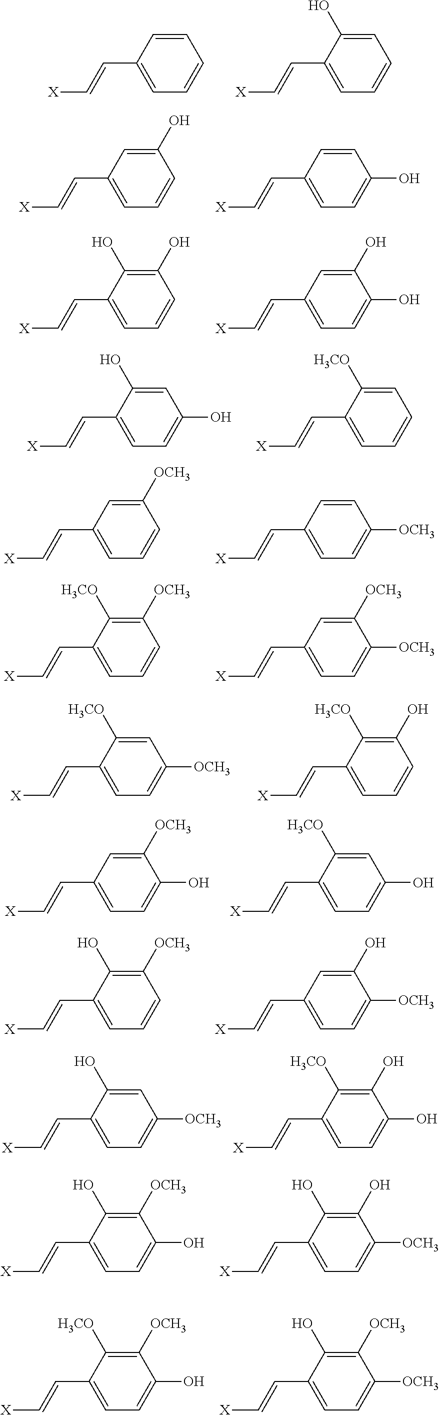

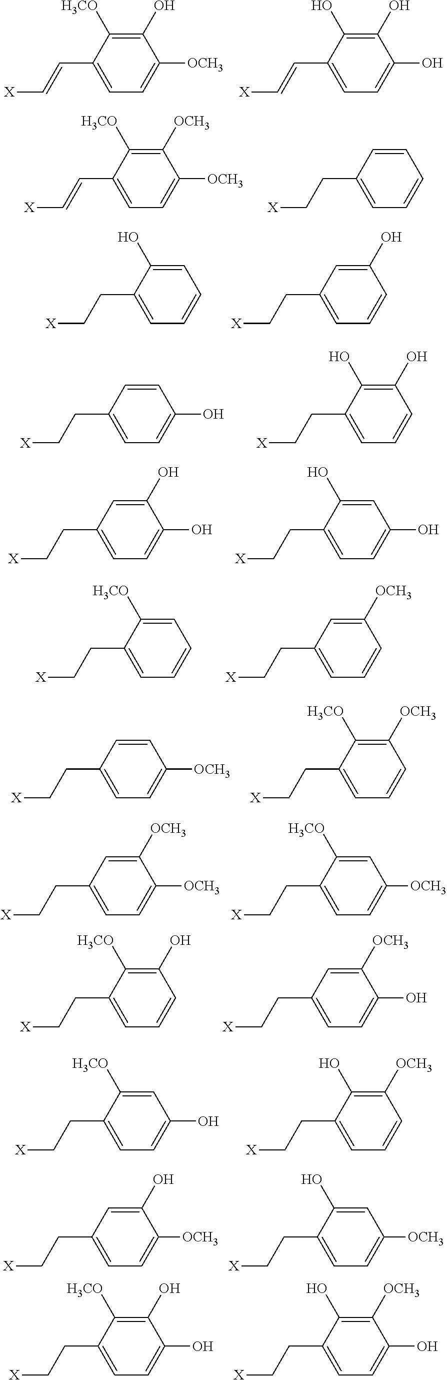

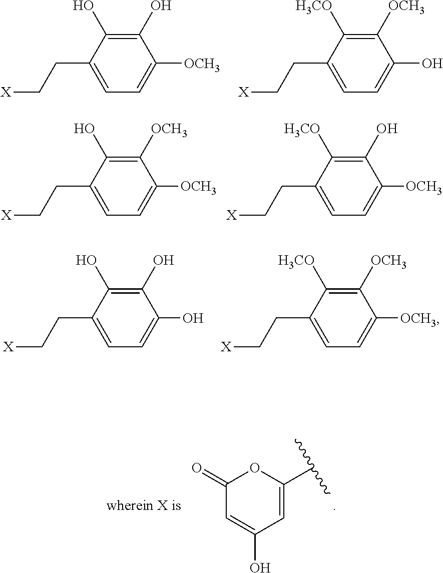

The methods to produce a compound of Formula (IV) include culturing cells engineered to express an enzyme that is at least 80% identical to PmOMT4 (SEQ ID NO: 5). The methods to produce a compound of Formula (IV) include culturing cells engineered to express an enzyme that is at least 80% identical to PmOMT1 (SEQ ID NO: 6). In certain embodiments, the enzyme is at least 80%, 70%, 75%, 80%, 85%, 90%, 95%, or 100% identical to PmOMT4 (SEQ ID NO: 5). In certain embodiments, the enzyme is at least 80%, 85%, 90%, 95%, or 100% identical to PmOMT1 (SEQ ID NO: 6). In certain embodiments, the enzyme is purified before reacting with a compound of Formula (III). In certain embodiments, the enzyme is partially purified before reacting with a compound of Formula (III).

In certain embodiments, the enzyme that is at least 80% identical to PmOMT4 (SEQ ID NO: 5) is a component in a fusion protein. In certain embodiments, the enzyme that is at least 80% identical to PmOMT1 (SEQ ID NO: 6) is a component in a fusion protein. A fusion protein may be created by joining two or more gene or gene segments that code for separate proteins. Translation of this fusion gene results in a single or multiple polypeptides with functional properties derived from each of the original proteins. A polyfunctional protein is a single protein that has at least two different activities, wherein that functionality is a native biological function or the result of an engineered enzyme fusion. Thus, a fusion protein may include multiple activities such as those described herein for the kavalactone or flavokavain pathway enzymes described herein (i.e., 4-coumarate-CoA ligase Pm4CL1 (at least 80% identical to SEQ ID NO: 1), styrylpyrone synthase PmSPS1 (at least 80% identical to SEQ ID NO: 2), PmSPS2 (at least 80% identical to SEQ ID NO: 3), PmCHS (at least 80% identical to SEQ ID NO: 4), methyltransferase PmOMT4 (at least 80% identical to SEQ ID NO: 5), methyltransferase PmOMT1 (at least 80% identical to SEQ ID NO: 6), cytochrome P450 enzyme PmMDB1 (at least 80% identical to SEQ ID NO: 7), and NADPH-dependent reductase PmRDCT10 (at least 80% identical to SEQ ID NO: 8)).

The enzyme that is at least 80% identical to PmOMT4 (SEQ ID NO: 5) is heterologous to the host cell. The enzyme that is at least 80% identical to PmOMT1 (SEQ ID NO: 6) is heterologous to the host cell. In certain embodiments, the enzyme that is at least 80% identical to PmOMT4 (SEQ ID NO: 5) is recombinantly produced. In certain embodiments, the enzyme that is at least 80% identical to PmOMT1 (SEQ ID NO: 6) is recombinantly produced. In certain embodiments, the enzyme that is at least 80% identical to PmOMT4 (SEQ ID NO: 5) is obtained from a wildtype organism. In certain embodiments, the enzyme that is at least 80% identical to PmOMT4 (SEQ ID NO: 5) is obtained from a mutant organism. In certain embodiments, the enzyme that is at least 80% identical to PmOMT4 (SEQ ID NO: 5) is obtained from a genetically-modified organism. In certain embodiments, the enzyme that is at least 80% identical to PmOMT1 (SEQ ID NO: 6) is obtained from a wildtype organism. In certain embodiments, the enzyme that is at least 80% identical to PmOMT1 (SEQ ID NO: 6) is obtained from a mutant organism. In certain embodiments, the enzyme that is at least 80% identical to PmOMT1 (SEQ ID NO: 6) is obtained from a genetically-modified organism. In certain embodiments, the organism is a non-human organism. In certain embodiments, the non-human organism is selected from group consisting of bacteria, yeast, and plant. In certain embodiments, the organism is a plant. In certain embodiments, the plant is Piper methysticum.

A nucleic acid encoding the enzyme may be introduced into the cell in a vector (e.g., plasmids, viral vectors, cosmids, and artificial chromosomes). In certain embodiments, the nucleic acid is cDNA derived from the amino acid sequence of the enzyme that is at least 80% identical to PmOMT4 (SEQ ID NO: 5). In certain embodiments, the nucleic acid is cDNA derived from the amino acid sequence of the enzyme that is at least 80% identical to PmOMT1 (SEQ ID NO: 6). In some embodiments multiple cDNAs comprising sequences complementary to different genes (e.g., 2, 3, 4, 5, or more genes) described herein (i.e., 4-coumarate-CoA ligase Pm4CL1 (at least 80% identical to SEQ ID NO: 1), styrylpyrone synthase PmSPS1 (at least 80% identical to SEQ ID NO: 2), PmSPS2 (at least 80% identical to SEQ ID NO: 3), PmCHS (at least 80% identical to SEQ ID NO: 4), methyltransferase PmOMT4 (at least 80% identical to SEQ ID NO: 5), methyltransferase PmOMT1 (at least 80% identical to SEQ ID NO: 6), cytochrome P450 enzyme PmMDB1 (at least 80% identical to SEQ ID NO: 7), and NADPH-dependent reductase PmRDCT10 (at least 80% identical to SEQ ID NO: 8)), are introduced into the same cell individually, or together, or as part of a single nucleic acid.

The host cells expressing the enzyme that is at least 80% identical to PmOMT4 (SEQ ID NO: 5) may be prokaryotic cells, such as bacterial cells (e.g., Escherichia coli cells), or eukaryotic cells, such as yeast cells or plant cells. The host cells expressing the enzyme that is at least 80% identical to PmOMT1 (SEQ ID NO: 6) may be prokaryotic cells, such as bacterial cells (e.g., Escherichia coli cells), or eukaryotic cells, such as yeast cells or plant cells. In certain embodiments, the host cell is capable of expressing two or more kavalactone or flavokavain pathway enzymes described herein. In certain embodiments, the host cell is a bacteria cell and is a wildtype, mutant, recombinant, or genetically engineered form of Escherichia coli. In certain embodiments, the host cell is a yeast cell and is a wildtype, mutant, recombinant, or genetically engineered form of Saccharomyces cerevisiae. In certain embodiments, the host cell is a plant cell and is a wildtype, mutant, recombinant, or genetically engineered form of Nicotiana benthamiana.

In certain embodiments, the method for producing a compound of Formula (IV) utilizes a compound of Formula (I), or a salt thereof, as the starting material and comprises the steps: condensing a compound of Formula (I), or a salt thereof, with coenzyme A (CoA) using an enzyme that is at least 80% identical to Pm4CL1 (SEQ ID NO: 1) to produce a compound of Formula (II); reacting a compound of Formula (II), or a salt thereof, with malonyl-CoA using an enzyme that is at least 80% identical to PmSPS1 (SEQ ID NO: 2) to produce a compound of Formula (III); and alkylating a compound of Formula (III), or a salt thereof, with S-adenosylmethionine using an enzyme that at least 80% identical to PmOMT4 (SEQ ID NO: 5) to produce a compound of Formula (IV).

In certain embodiments, the method for producing a compound of Formula (IV) utilizes a compound of Formula (I), or a salt thereof, as the starting material and comprises the steps: condensing a compound of Formula (I), or a salt thereof, with coenzyme A (CoA) using an enzyme that is at least 80% identical to Pm4CL1 (SEQ ID NO: 1) to produce a compound of Formula (II); reacting a compound of Formula (II), or a salt thereof, with malonyl-CoA using an enzyme that is at least 80% identical to PmSPS1 (SEQ ID NO: 2) to produce a compound of Formula (III); and alkylating a compound of Formula (III), or a salt thereof, with S-adenosylmethionine using an enzyme that at least 80% identical to PmOMT1 (SEQ ID NO: 6) to produce a compound of Formula (IV).

In certain embodiments, the method for producing a compound of Formula (IV) utilizes a compound of Formula (I), or a salt thereof, as the starting material and comprises the steps: condensing a compound of Formula (I), or a salt thereof, with coenzyme A (CoA) using an enzyme that is at least 80% identical to Pm4CL1 (SEQ ID NO: 1) to produce a compound of Formula (II); reacting a compound of Formula (II), or a salt thereof, with malonyl-CoA using an enzyme that is at least 80% identical to PmSPS2 (SEQ ID NO: 3) to produce a compound of Formula (III); and alkylating a compound of Formula (III), or a salt thereof, with S-adenosylmethionine using an enzyme that at least 80% identical to PmOMT4 (SEQ ID NO: 5) to produce a compound of Formula (IV).

In certain embodiments, the method for producing a compound of Formula (IV) utilizes a compound of Formula (I), or a salt thereof, as the starting material and comprises the steps: condensing a compound of Formula (I), or a salt thereof, with coenzyme A (CoA) using an enzyme that is at least 80% identical to Pm4CL1 (SEQ ID NO: 1) to produce a compound of Formula (II); reacting a compound of Formula (II), or a salt thereof, with malonyl-CoA using an enzyme that is at least 80% identical to PmSPS2 (SEQ ID NO: 3) to produce a compound of Formula (III); and alkylating a compound of Formula (III), or a salt thereof, with S-adenosylmethionine using an enzyme that at least 80% identical to PmOMT1 (SEQ ID NO: 6) to produce a compound of Formula (IV).

### Production of 6-styryl-4-hydroxyl-5,6-dihydro-2-pyrone Compounds of Formula (V)

Some aspects of the present disclosure provides methods for producing a compound of Formula (V) from a compound of Formula (IV), or a salt thereof, and a reducing agent (i.e., NADPH or NADH) using an enzyme that is at least 80% identical to PmRDCT10 (SEQ ID NO: 8), wherein:

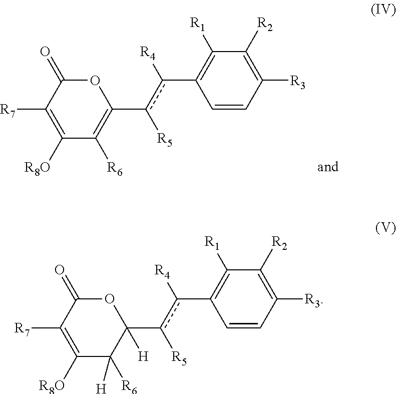

In certain, embodiments, the reduction reaction of a compound of Formula (IV) with NADPH to produce a compound of Formula (V) occurs in vitro. In certain embodiments, the reduction reaction of a compound of Formula (IV) with NADPH to produce a compound of Formula (V) occurs in vivo. In certain, embodiments, the reduction reaction of a compound of Formula (IV) with NADH to produce a compound of Formula (V) occurs in vitro. In certain embodiments, the reduction reaction of a compound of Formula (IV) with NADH to produce a compound of Formula (V) occurs in vivo.

In certain embodiments, [symbol] is a single bond. In certain embodiments, [symbol] is a double bond.

In certain embodiments, each of R₁, R₂, R₃, R₆, and R₇ independently is hydrogen, optionally substituted, cyclic or acyclic aliphatic, or ORₓ, wherein Rₓ is hydrogen or optionally substituted, cyclic or acyclic aliphatic. In certain embodiments, R₁ is hydrogen. In certain embodiments, R₂ is hydrogen. In certain embodiments, R₃ is hydrogen. In certain embodiments, R₁ is —OH. In certain embodiments, R₂ is OH. In certain embodiments, R₃ is —OH. In certain embodiments, R₁ is —OCH₃. In certain embodiments, R₂ is —OCH₃. In certain embodiments, R₃ is —OCH₃. In certain embodiments, R₁, R₂, and R₃ are hydrogen. In certain embodiments, R₁, R₂, and R₃ are —OH. In certain embodiments, R₁ and R₃ are —OH. In certain embodiments, R₂ and R₃ are —OH. In certain embodiments, R₂ is —OCH₃. In certain embodiments, R₆ is hydrogen. In certain embodiments, R₆ is —OH. In certain embodiments, R₇ is hydrogen. In certain embodiments, R₆ is —OH and R₇ is hydrogen. In certain embodiments, both R₆ and R₇ are hydrogen. In certain embodiments, R₈ is hydrogen. In certain embodiments, wherein R₈ is —CH₃. R₁, R₂, and R₃ are —OCH₃. In certain embodiments, R₁ and R₃ are —OCH₃. In certain embodiments, R₂ and R₃ are —OCH₃. In certain embodiments, R₂ and R₃ are —OCH₃ and R₈ is —CH₃. In certain embodiments, R₂ and R₃ are —OH and R₈ is —H.

In certain embodiments, each of R₄ and R₅ independently is hydrogen or optionally substituted, cyclic or acyclic aliphatic. In certain embodiments, R₄ and R₅ are hydrogen.

The methods to produce a compound of Formula (V) include reacting NADPH or NADH with a compound of Formula (IV) selected from the group consisting of:

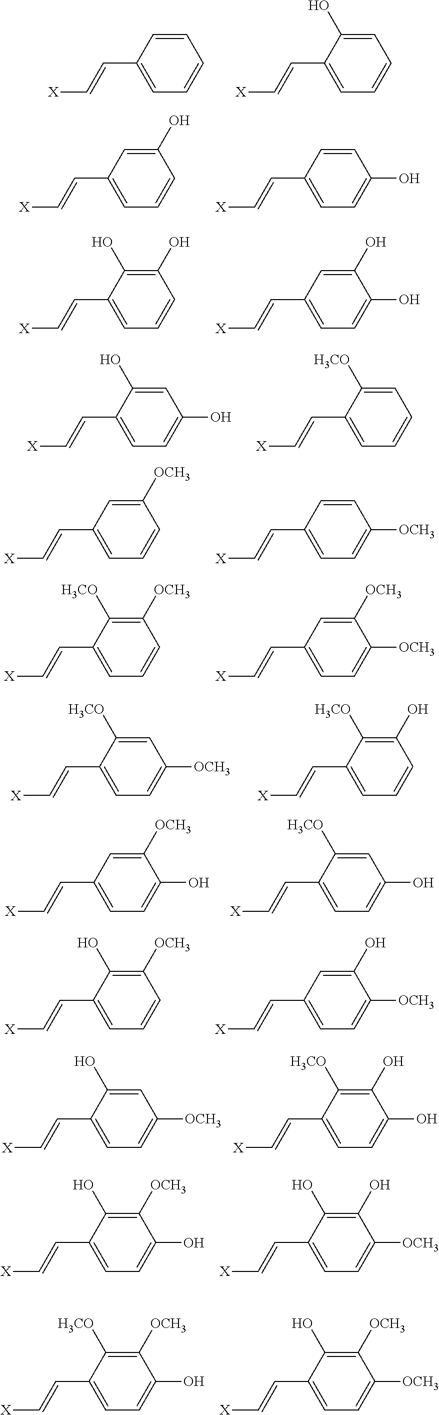

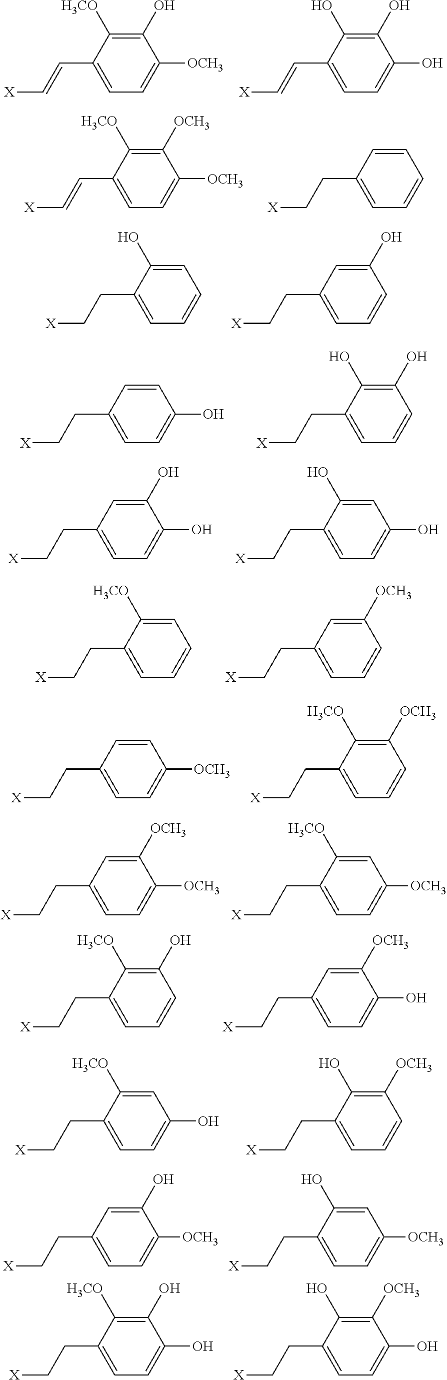

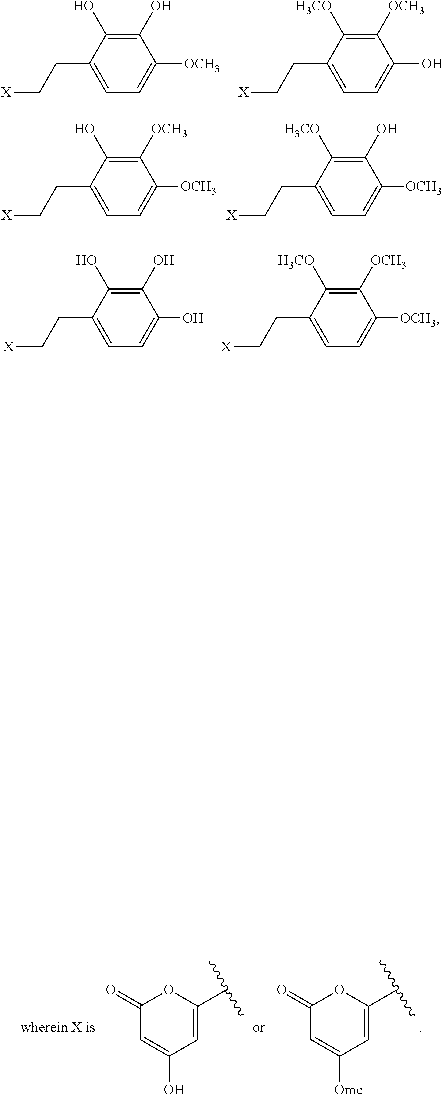

The methods to produce a compound of Formula (V) include culturing cells engineered to express an enzyme that is at least 80% identical to PmRDCT10 (SEQ ID NO: 8). In certain embodiments, the enzyme is at least 80%, 85%, 90%, 95%, or 100% identical to PmRDCT10 (SEQ ID NO: 8). In certain embodiments, the enzyme is purified before reacting with a compound of Formula (IV). In certain embodiments, the enzyme is partially purified before reacting with a compound of Formula (IV).

In certain embodiments, the enzyme that is at least 80% identical to PmRDCT10 (SEQ ID NO: 8) is a component in a fusion protein. A fusion protein may be created by joining two or more gene or gene segments that code for separate proteins. Translation of this fusion gene results in a single or multiple polypeptides with functional properties derived from each of the original proteins. A polyfunctional protein is a single protein that has at least two different activities, wherein that functionality is a native biological function or the result of an engineered enzyme fusion. Thus, a fusion protein may include multiple activities such as those described herein for the kavalactone or flavokavain pathway enzymes described herein (i.e., 4-coumarate-CoA ligase Pm4CL1 (at least 80% identical to SEQ ID NO: 1), styrylpyrone synthase PmSPS1 (at least 80% identical to SEQ ID NO: 2), PmSPS2 (at least 80% identical to SEQ ID NO: 3), PmCHS (at least 80% identical to SEQ ID NO: 4), methyltransferase PmOMT4 (at least 80% identical to SEQ ID NO: 5), methyltransferase PmOMT1 (at least 80% identical to SEQ ID NO: 6), cytochrome P450 enzyme PmMDB1 (at least 80% identical to SEQ ID NO: 7), and NADPH-dependent reductase PmRDCT10 (at least 80% identical to SEQ ID NO: 8)).

The enzyme that is at least 80% identical to PmRDCT10 (SEQ ID NO: 8) is heterologous to the host cell. In certain embodiments, the enzyme that is at least 80% identical to PmRDCT10 (SEQ ID NO: 8) is recombinantly produced. In certain embodiments, the enzyme that is at least 80% identical to PmRDCT10 (SEQ ID NO: 8) is obtained from a wildtype organism. In certain embodiments, the enzyme that is at least 80% identical to PmRDCT10 (SEQ ID NO: 8) is obtained from a mutant organism. In certain embodiments, the enzyme that is at least 80% identical to PmRDCT10 (SEQ ID NO: 8) is obtained from a genetically-modified organism. In certain embodiments, the organism is a non-human organism. In certain embodiments, the non-human organism is selected from group consisting of bacteria, yeast, and plant. In certain embodiments, the organism is a plant. In certain embodiments, the plant is Piper methysticum.

A nucleic acid encoding the enzyme may be introduced into the cell in a vector (e.g., plasmids, viral vectors, cosmids, and artificial chromosomes). In certain embodiments, the nucleic acid is cDNA derived from the amino acid sequence of the enzyme that is at least 80% identical to PmRDCT10 (SEQ ID NO: 8). In some embodiments multiple cDNAs comprising sequences complementary to different genes (e.g., 2, 3, 4, 5, or more genes) described herein (i.e., 4-coumarate-CoA ligase Pm4CL1 (at least 80% identical to SEQ ID NO: 1), styrylpyrone synthase PmSPS1 (at least 80% identical to SEQ ID NO: 2), PmSPS2 (at least 80% identical to SEQ ID NO: 3), PmCHS (at least 80% identical to SEQ ID NO: 4), methyltransferase PmOMT4 (at least 80% identical to SEQ ID NO: 5), methyltransferase PmOMT1 (at least 80% identical to SEQ ID NO: 6), cytochrome P450 enzyme PmMDB1 (at least 80% identical to SEQ ID NO: 7), and NADPH-dependent reductase PmRDCT10 (at least 80% identical to SEQ ID NO: 8)), are introduced into the same cell individually, or together, or as part of a single nucleic acid.

The host cells expressing the enzyme that is at least 80% identical to PmRDCT10 (SEQ ID NO: 8) may be prokaryotic cells, such as bacterial cells (e.g., Escherichia coli cells), or eukaryotic cells, such as yeast cells or plant cells. In certain embodiments, the host cell is capable of expressing two or more kavalactone or flavokavain pathway enzymes described herein. In certain embodiments, the host cell is a bacteria cell and is a wildtype, mutant, recombinant, or genetically engineered form of Escherichia coli. In certain embodiments, the host cell is a yeast cell and is a wildtype, mutant, recombinant, or genetically engineered form of Saccharomyces cerevisiae. In certain embodiments, the host cell is a plant cell and is a wildtype, mutant, recombinant, or genetically engineered form of Nicotiana benthamiana.

### Production of 6-styryl-4-hydroxyl-2-pyrone Compounds with a Methylenedioxy Bridge of Formula (VI)

Some aspects of the present disclosure provides methods for producing a compound of Formula (VI) from a compound of Formula (IV), or a salt thereof, and a reducing agent (i.e., NADPH or NADH) using an enzyme that is at least 80% identical to PmMDB1 (SEQ ID NO: 7), wherein:

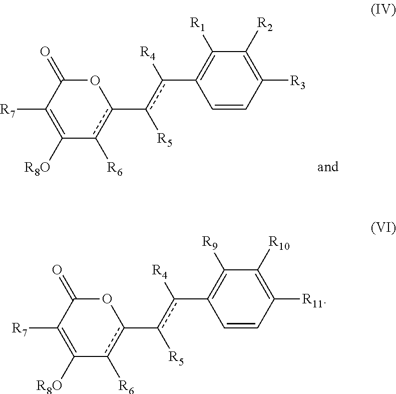

In certain, embodiments, the reduction reaction of a compound of Formula (IV) with NADPH to produce a compound of Formula (VI) occurs in vitro. In certain embodiments, the reduction reaction of a compound of Formula (IV) with NADPH to produce a compound of Formula (VI) occurs in vivo. In certain, embodiments, the reduction reaction of a compound of Formula (IV) with NADH to produce a compound of Formula (VI) occurs in vitro. In certain embodiments, the reduction reaction of a compound of Formula (IV) with NADH to produce a compound of Formula (VI) occurs in vivo.

In certain embodiments, [symbol] is a single bond. In certain embodiments, [symbol] is a double bond.

In certain embodiments, each of R₁, R₂, R₃, R₆, R₇, R₉, R₁₀, and R₁₁ independently is hydrogen, optionally substituted, cyclic or acyclic aliphatic, or ORₓ, or R₉ and R₁₀ are combined to form a ring, or R₁₀ and R₁₁ are combined to form a ring, wherein Rₓ is hydrogen or optionally substituted, cyclic or acyclic aliphatic. In certain embodiments, R₁ is hydrogen. In certain embodiments, R₂ is hydrogen. In certain embodiments, R₃ is hydrogen. In certain embodiments, R₁ is —OH. In certain embodiments, R₂ is —OH. In certain embodiments, R₃ is —OH. In certain embodiments, R₁ is —OCH₃. In certain embodiments, R₂ is —OCH₃. In certain embodiments, R₃ is —OCH₃. In certain embodiments, R₁, R₂, and R₃ are hydrogen. In certain embodiments, R₁, R₂, and R₃ are —OH. In certain embodiments, R₁ and R₃ are —OH. In certain embodiments, R₂ and R₃ are —OH. In certain embodiments, R₂ is —OCH₃. In certain embodiments, R₆ is hydrogen. In certain embodiments, R₆ is —OH. In certain embodiments, R₇ is hydrogen. In certain embodiments, R₆ is —OH and R₇ is hydrogen. In certain embodiments, both R₆ and R₇ are hydrogen. In certain embodiments, R₈ is hydrogen. In certain embodiments, wherein R₈ is —CH₃. In certain embodiments, R₉ and R₁₀ are combined to form a methylenedioxy cyclic moiety —OCH₂O— and R₁₁ is hydrogen. In certain embodiments, R₉ and R₁₀ are combined to form a methylenedioxy cyclic moiety —OCH₂O— and R₁₁ is optionally substituted, cyclic or acyclic aliphatic. In certain embodiments, R₉ and R₁₀ are combined to form a methylenedioxy cyclic moiety —OCH₂O— and R₁₁ is —OH. In certain embodiments, R₉ and R₁₀ are combined to form a methylenedioxy cyclic moiety —OCH₂O— and R₁₁ is —OCH₃. In certain embodiments, R₁₀ and R₁₁ are combined to form a methylenedioxy cyclic moiety —OCH₂O— and R₉ is hydrogen. In certain embodiments, R₁₀ and R₁₁ are combined to form a methylenedioxy cyclic moiety —OCH₂O— and R₉ is optionally substituted, cyclic or acyclic aliphatic. In certain embodiments, R₁₀ and R₁₁ are combined to form a methylenedioxy cyclic moiety —OCH₂O— and R₉ is —OH. In certain embodiments, R₁₀ and R₁₁ are combined to form a methylenedioxy cyclic moiety —OCH₂O— and R₉ is —OCH₃. In certain embodiments, R₁ is —OH, R₂ is —OCH₃, and R₉ and R₁₀ are combined to form a methylenedioxy cyclic moiety —OCH₂O—. In certain embodiments, R₁ is —OCH₃, R₂ is —OH, and R₉ and R₁₀ are combined to form a methylenedioxy cyclic moiety —OCH₂O—. In certain, embodiments, R₂ is —OH, R₃ is —OCH₃, and R₁₀ and R₁₁ are combined to form a methylenedioxy cyclic moiety —OCH₂O—. In certain embodiments, R₂ is —OCH₃, R₃ is —OH, and R₁₀ and R₁₁ are combined to form a methylenedioxy cyclic moiety —OCH₂O—.

In certain embodiments, each of R₄ and R₅ independently is hydrogen or optionally substituted, cyclic or acyclic aliphatic. In certain embodiments, R₄ and R₅ are hydrogen.

The methods to produce a compound of Formula (VI) include reacting NADPH or NADH with a compound of Formula (IV) selected from the group consisting of:

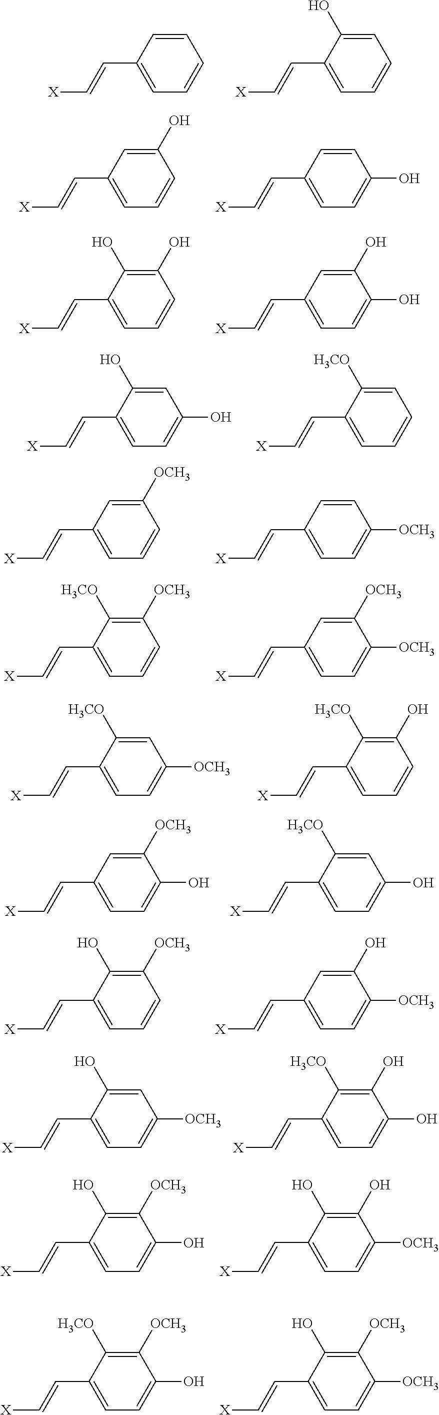

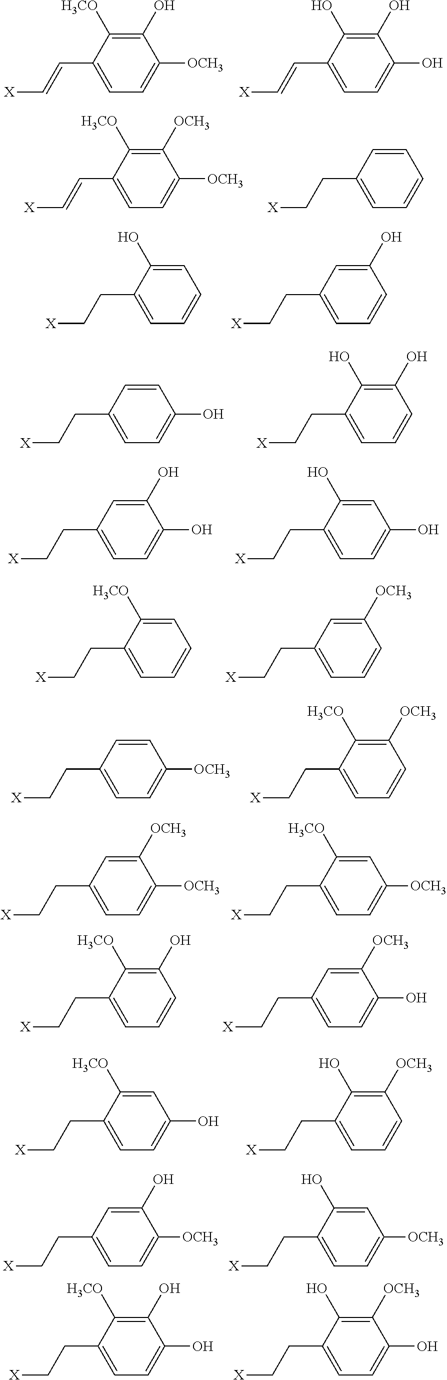

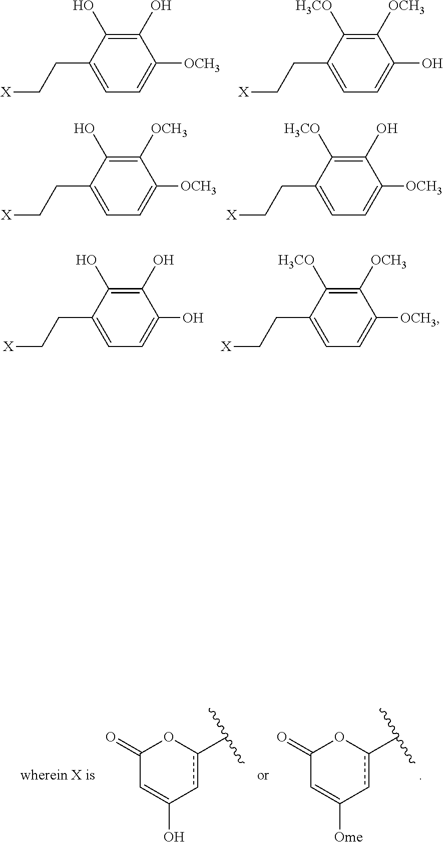

The methods to produce a compound of Formula (VI) include culturing cells engineered to express an enzyme that is at least 80% identical to PmMDB1 (SEQ ID NO: 7). In certain embodiments, the enzyme is at least 80%, 85%, 90%, 95%, or 100% identical to PmMDB1 (SEQ ID NO: 7). In certain embodiments, the enzyme is purified before reacting with a compound of Formula (IV). In certain embodiments, the enzyme is partially purified before reacting with a compound of Formula (IV).

In certain embodiments, the enzyme that is at least 80% identical to PmMDB1 (SEQ ID NO: 7) is a component in a fusion protein. A fusion protein may be created by joining two or more gene or gene segments that code for separate proteins. Translation of this fusion gene results in a single or multiple polypeptides with functional properties derived from each of the original proteins. A polyfunctional protein is a single protein that has at least two different activities, wherein that functionality is a native biological function or the result of an engineered enzyme fusion. Thus, a fusion protein may include multiple activities such as those described herein for the kavalactone or flavokavain pathway enzymes described herein (i.e., 4-coumarate-CoA ligase Pm4CL1 (at least 80% identical to SEQ ID NO: 1), styrylpyrone synthase PmSPS1 (at least 80% identical to SEQ ID NO: 2), PmSPS2 (at least 80% identical to SEQ ID NO: 3), PmCHS (at least 80% identical to SEQ ID NO: 4), methyltransferase PmOMT4 (at least 80% identical to SEQ ID NO: 5), methyltransferase PmOMT1 (at least 80% identical to SEQ ID NO: 6), cytochrome P450 enzyme PmMDB1 (at least 80% identical to SEQ ID NO: 7), and NADPH-dependent reductase PmRDCT10 (at least 80% identical to SEQ ID NO: 8)).

The enzyme that is at least 80% identical to PmMDB1 (SEQ ID NO: 7) is heterologous to the host cell. In certain embodiments, the enzyme that is at least 80% identical to PmMDB1 (SEQ ID NO: 7) is recombinantly produced. In certain embodiments, the enzyme that is at least 80% identical to PmMDB1 (SEQ ID NO: 7) is obtained from a wildtype organism. In certain embodiments, the enzyme that is at least 80% identical to PmMDB1 (SEQ ID NO: 7) is obtained from a mutant organism. In certain embodiments, the enzyme that is at least 80% identical to PmMDB1 (SEQ ID NO: 7) is obtained from a genetically-modified organism. In certain embodiments, the organism is a non-human organism. In certain embodiments, the non-human organism is selected from group consisting of bacteria, yeast, and plant. In certain embodiments, the organism is a plant. In certain embodiments, the plant is Piper methysticum.

A nucleic acid encoding the enzyme may be introduced into the cell in a vector (e.g., plasmids, viral vectors, cosmids, and artificial chromosomes). In certain embodiments, the nucleic acid is cDNA derived from the amino acid sequence of the enzyme that is at least 80% identical to PmMDB1 (SEQ ID NO: 7). In some embodiments multiple cDNAs comprising sequences complementary to different genes (e.g., 2, 3, 4, 5, or more genes) described herein (i.e., 4-coumarate-CoA ligase Pm4CL1 (at least 80% identical to SEQ ID NO: 1), styrylpyrone synthase PmSPS1 (at least 80% identical to SEQ ID NO: 2), PmSPS2 (at least 80% identical to SEQ ID NO: 3), PmCHS (at least 80% identical to SEQ ID NO: 4), methyltransferase PmOMT4 (at least 80% identical to SEQ ID NO: 5), methyltransferase PmOMT1 (at least 80% identical to SEQ ID NO: 6), cytochrome P450 enzyme PmMDB1 (at least 80% identical to SEQ ID NO: 7), and NADPH-dependent reductase PmRDCT10 (at least 80% identical to SEQ ID NO: 8)), are introduced into the same cell individually, or together, or as part of a single nucleic acid.

The host cells expressing the enzyme that is at least 80% identical to PmMDB1 (SEQ ID NO: 7) may be prokaryotic cells, such as bacterial cells (e.g., Escherichia coli cells), or eukaryotic cells, such as yeast cells or plant cells. In certain embodiments, the host cell is capable of expressing two or more kavalactone or flavokavain pathway enzymes described herein. In certain embodiments, the host cell is a bacteria cell and is a wildtype, mutant, recombinant, or genetically engineered form of Escherichia coli. In certain embodiments, the host cell is a yeast cell and is a wildtype, mutant, recombinant, or genetically engineered form of Saccharomyces cerevisiae. In certain embodiments, the host cell is a plant cell and is a wildtype, mutant, recombinant, or genetically engineered form of Nicotiana benthamiana.

### Production of Chalcone Compounds of Formula (VII)

Some aspects of the present disclosure provides methods for producing a compound of Formula (VII) from a compound of Formula (II), or a salt thereof, and malonyl-CoA using an enzyme that is at least 80% identical to PmCHS (SEQ ID NO: 4), wherein:

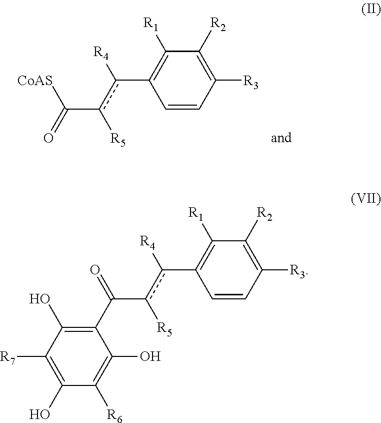

In certain embodiments, the reaction of a compound of Formula (II) with malonyl-CoA to produce a compound of Formula (VII) utilizes three or more molar equivalents of malonyl-CoA relative to the compound of Formula (II). In certain embodiments, the reaction of a compound of Formula (II) with malonyl-CoA to produce a compound of Formula (VII) occurs in vitro. In certain embodiments, the reaction of a compound of Formula (II) with malonyl-CoA to produce a compound of Formula (VII) occurs in vivo.

In certain embodiments, [symbol] is a single bond. In certain embodiments, [symbol] is a double bond.

In certain embodiments, each of R₁, R₂, R₃, R₆, and R₇ independently is hydrogen, optionally substituted, cyclic or acyclic aliphatic, or ORₓ, wherein Rₓ is hydrogen or optionally substituted, cyclic or acyclic aliphatic. In certain embodiments, R₁ is hydrogen. In certain embodiments, R₂ is hydrogen. In certain embodiments, R₃ is hydrogen. In certain embodiments, R₁ is —OH. In certain embodiments, R₂ is —OH. In certain embodiments, R₃ is —OH. In certain embodiments, R₁ is —OCH₃. In certain embodiments, R₂ is —OCH₃. In certain embodiments, R₃ is —OCH₃. In certain embodiments, R₁, R₂, and R₃ are hydrogen. In certain embodiments, R₁ and R₂ are hydrogen and R₃ is —OH. In certain embodiments, R₁ and R₂ are hydrogen and R₃ is —OCH₃. In certain embodiments, R₆ is hydrogen. In certain embodiments, R₇ is hydrogen.

In certain embodiments, each of R₄ and R₅ independently is hydrogen or optionally substituted, cyclic or acyclic aliphatic. In certain embodiments, R₄ and R₅ are hydrogen.

The methods to produce a compound of Formula (VII) include reacting malonyl-CoA with a compound of Formula (II) selected from the group consisting of:

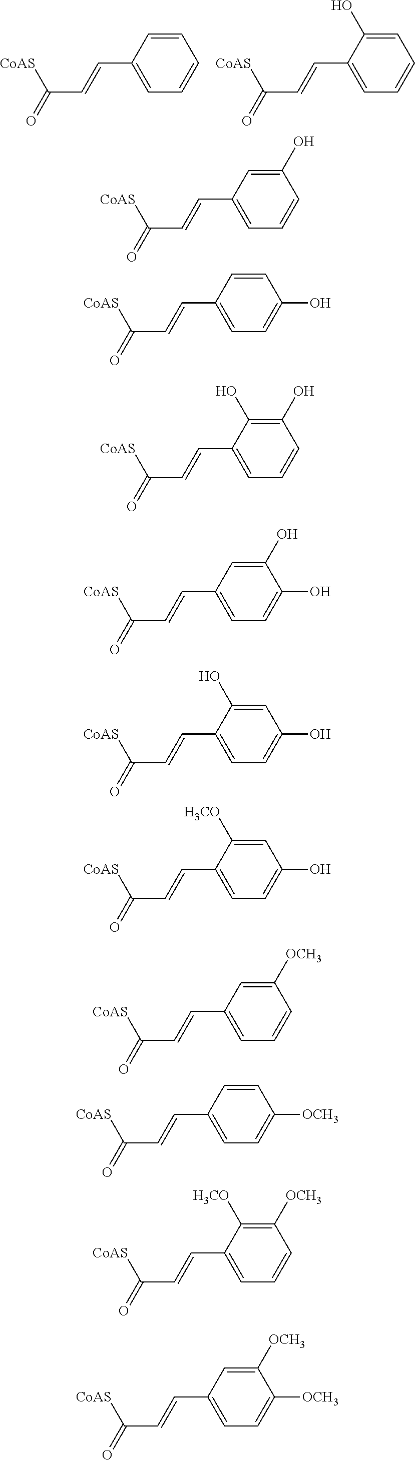

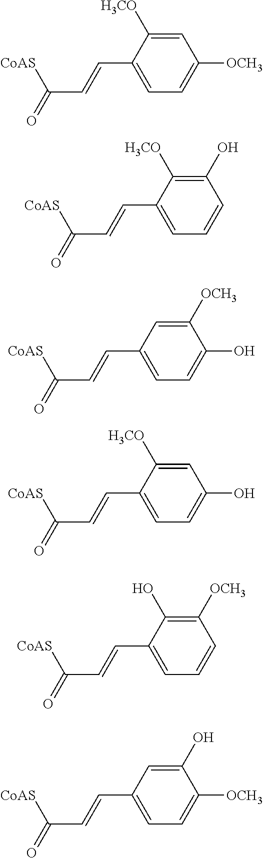

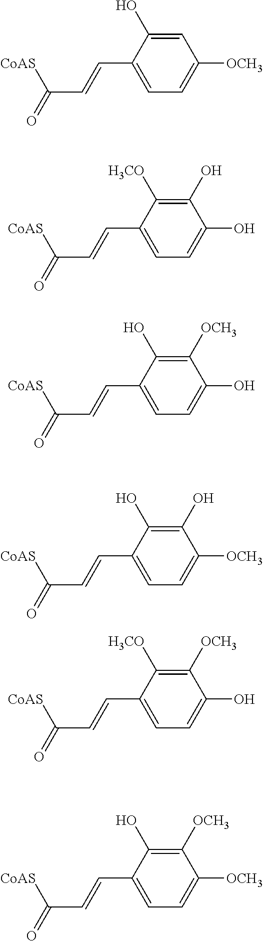

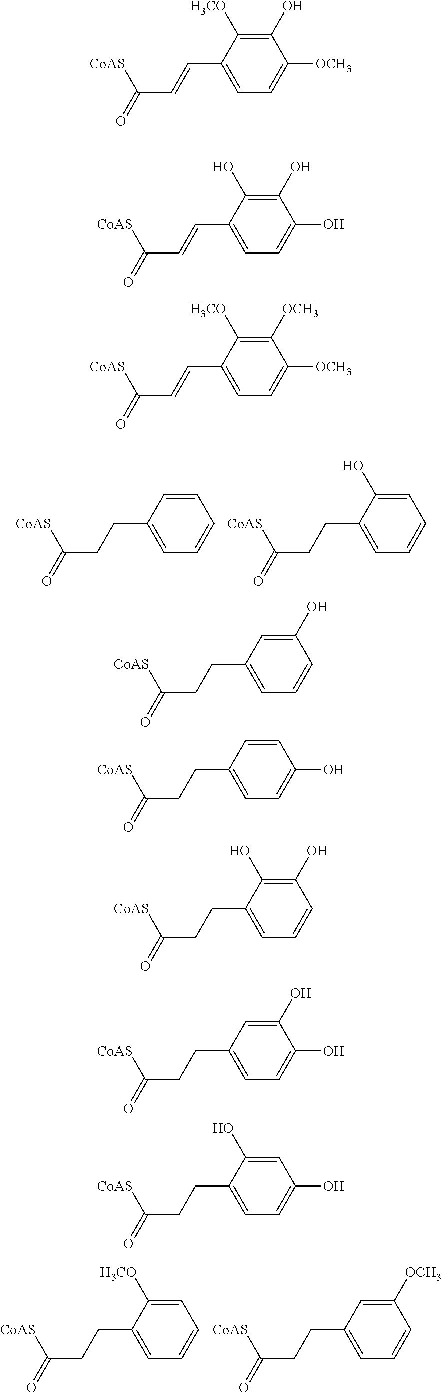

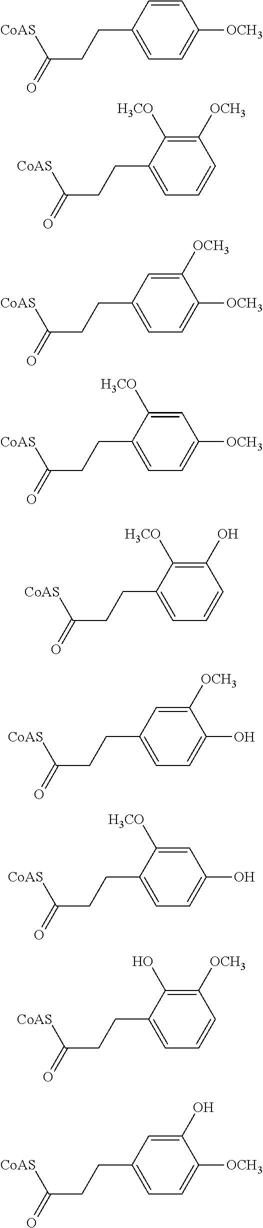

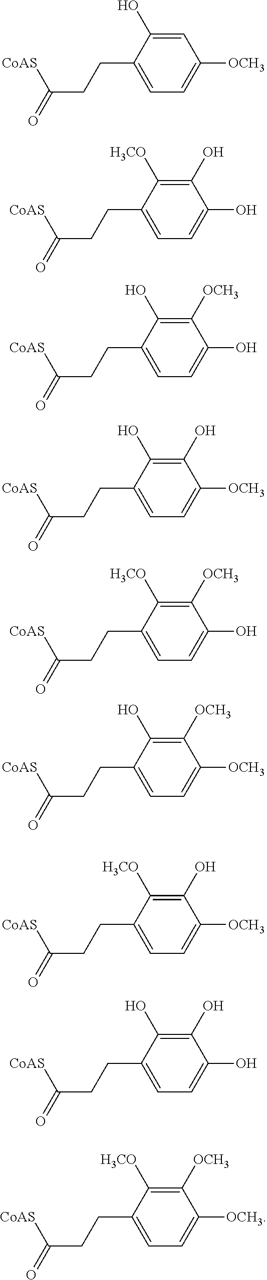

The methods to produce a compound of Formula (VII) include culturing cells engineered to express an enzyme that is at least 80% identical to PmCHS (SEQ ID NO: 4). In certain embodiments, the enzyme is at least 80%, 85%, 90%, 95%, or 100% identical to PmCHS (SEQ ID NO: 4). In certain embodiments, the enzyme is purified before reacting with a compound of Formula (II). In certain embodiments, the enzyme is partially purified before reacting with a compound of Formula (II).

In certain embodiments, the enzyme that is at least 80% identical to PmCHS (SEQ ID NO: 4) is a component in a fusion protein. A fusion protein may be created by joining two or more gene or gene segments that code for separate proteins. Translation of this fusion gene results in a single or multiple polypeptides with functional properties derived from each of the original proteins. A polyfunctional protein is a single protein that has at least two different activities, wherein that functionality is a native biological function or the result of an engineered enzyme fusion. Thus, a fusion protein may include multiple activities such as those described herein for the kavalactone or flavokavain pathway enzymes described herein (i.e., 4-coumarate-CoA ligase Pm4CL1 (at least 80% identical to SEQ ID NO: 1), styrylpyrone synthase PmSPS1 (at least 80% identical to SEQ ID NO: 2), PmSPS2 (at least 80% identical to SEQ ID NO: 3), PmCHS (at least 80% identical to SEQ ID NO: 4), methyltransferase PmOMT4 (at least 80% identical to SEQ ID NO: 5), methyltransferase PmOMT1 (at least 80% identical to SEQ ID NO: 6), cytochrome P450 enzyme PmMDB1 (at least 80% identical to SEQ ID NO: 7), and NADPH-dependent reductase PmRDCT10 (at least 80% identical to SEQ ID NO: 8)).

The enzyme that is at least 80% identical to PmCHS (SEQ ID NO: 4) is heterologous to the host cell. In certain embodiments, the enzyme that is at least 80% identical to PmCHS (SEQ ID NO: 4) is recombinantly produced. In certain embodiments, the enzyme that is at least 80% identical to PmCHS (SEQ ID NO: 4) is obtained from a wildtype organism. In certain embodiments, the enzyme that is at least 80% identical to PmCHS (SEQ ID NO: 4) is obtained from a mutant organism. In certain embodiments, the enzyme that is at least 80% identical to PmCHS (SEQ ID NO: 4) is obtained from a genetically-modified organism. In certain embodiments, the organism is a non-human organism. In certain embodiments, the non-human organism is selected from group consisting of bacteria, yeast, and plant. In certain embodiments, the organism is a plant. In certain embodiments, the plant is Piper methysticum.

A nucleic acid encoding the enzyme may be introduced into the cell in a vector (e.g., plasmids, viral vectors, cosmids, and artificial chromosomes). In certain embodiments, the nucleic acid is cDNA derived from the amino acid sequence of the enzyme that is at least 80% identical to PmCHS (SEQ ID NO: 4). In some embodiments multiple cDNAs comprising sequences complementary to different genes (e.g., 2, 3, 4, 5, or more genes) described herein (i.e., 4-coumarate-CoA ligase Pm4CL1 (at least 80% identical to SEQ ID NO: 1), styrylpyrone synthase PmSPS1 (at least 80% identical to SEQ ID NO: 2), PmSPS2 (at least 80% identical to SEQ ID NO: 3), PmCHS (at least 80% identical to SEQ ID NO: 4), methyltransferase PmOMT4 (at least 80% identical to SEQ ID NO: 5), methyltransferase PmOMT1 (at least 80% identical to SEQ ID NO: 6), cytochrome P450 enzyme PmMDB1 (at least 80% identical to SEQ ID NO: 7), and NADPH-dependent reductase PmRDCT10 (at least 80% identical to SEQ ID NO: 8)), are introduced into the same cell individually, or together, or as part of a single nucleic acid.

The host cells expressing the enzyme that is at least 80% identical to PmCHS (SEQ ID NO: 4) may be prokaryotic cells, such as bacterial cells (e.g., Escherichia coli cells), or eukaryotic cells, such as yeast cells or plant cells. In certain embodiments, the host cell is capable of expressing two or more kavalactone or flavokavain pathway enzymes described herein. In certain embodiments, the host cell is a bacteria cell and is a wildtype, mutant, recombinant, or genetically engineered form of Escherichia coli. In certain embodiments, the host cell is a yeast cell and is a wildtype, mutant, recombinant, or genetically engineered form of Saccharomyces cerevisiae. In certain embodiments, the host cell is a plant cell and is a wildtype, mutant, recombinant, or genetically engineered form of Nicotiana benthamiana.

In certain embodiments, the method for producing a compound of Formula (VII) utilizes a compound of Formula (I), or a salt thereof, as the starting material and comprises the steps: condensing a compound of Formula (I), or a salt thereof, with coenzyme A (CoA) using an enzyme that is at least 80% identical to Pm4CL1 (SEQ ID NO: 1) to produce a compound of Formula (II); and reacting a compound of Formula (II), or a salt thereof, with malonyl-CoA using an enzyme that is at least 80% identical to PmCHS (SEQ ID NO: 4) to produce a compound of Formula (VII).

### Production of Methylated Chalcone Compounds of Formula (VIII)

Some aspects of the present disclosure provides methods for producing a compound of Formula (VIII) from a compound of Formula (VII), or a salt thereof, and S-adenosylmethionine using an enzyme that is at least 80% identical to PmOMT4 (SEQ ID NO: 5). Some aspects of the present disclosure provides methods for producing a compound of Formula (VIII) from a compound of Formula (VII), or a salt thereof, and S-adenosylmethionine using an enzyme that is at least 80% identical to PmOMT1 (SEQ ID NO: 6). The structure of a compound of Formula (VII) and a structure of a compound of Formula (VIII) are as follows:

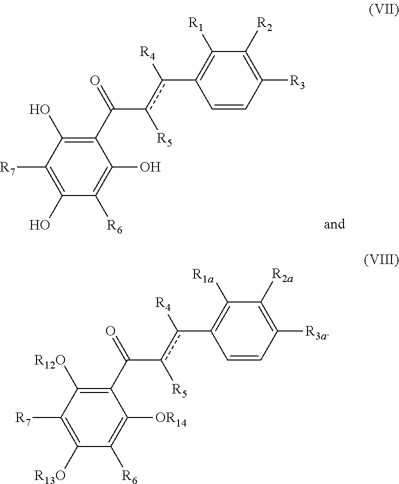

In certain embodiments, the reaction of a compound of Formula (VII) with S-adenosylmethionine to produce a compound of Formula (VIII) occurs in vitro. In certain, embodiments, the reaction of a compound of Formula (VII) with S-adenosylmethionine to produce a compound of Formula (VIII) occurs in vivo.

In certain embodiments, [symbol] is a single bond. In certain embodiments, [symbol] is a double bond.

In certain embodiments, each of R₁, R₂, R₃, R₁ₐ, R₂ₐ, R₃ₐ, R₆, and R₇ independently is hydrogen, optionally substituted, cyclic or acyclic aliphatic, or ORₓ, wherein Rₓ is hydrogen or optionally substituted, cyclic or acyclic aliphatic. In certain embodiments, R₁ is hydrogen. In certain embodiments, R₂ is hydrogen. In certain embodiments, R₃ is hydrogen. In certain embodiments, R₁ is —OH. In certain embodiments, R₂ is OH. In certain embodiments, R₃ is —OH. In certain embodiments, R₁ is —OCH₃. In certain embodiments, R₂ is —OCH₃. In certain embodiments, R₃ is —OCH₃. In certain embodiments, R₁, R₂, and R₃ are hydrogen. In certain embodiments, R₁, R₂, and R₃ are —OH. In certain embodiments, R₁ and R₃ are —OH. In certain embodiments, R₂ and R₃ are —OH. In certain embodiments, R₂ is —OCH₃. In certain embodiments, R₆ is hydrogen. In certain embodiments, R₇ is hydrogen. In certain embodiments, both R₆ and R₇ are hydrogen.

In certain embodiments, each of R₄ and R₅ independently is hydrogen or optionally substituted, cyclic or acyclic aliphatic. In certain embodiments, R₄ and R₅ are hydrogen.

In certain embodiments, each of R₁₂ and R₁₃ independently is optionally substituted, cyclic or acyclic aliphatic. In certain embodiments, R₁₂ and R₁₃ are —CH₃. In certain embodiments, R₁₄ is hydrogen. In certain embodiments, R₁₄ and R₁₃ are —CH₃. In certain embodiments, R₁₂ is hydrogen. In certain embodiments, R₁₂ is —CH₃, R₁₃ is —CH₃, and R₁₄ is hydrogen.

In certain embodiments, R₁ₐ, R₂ₐ, and R₃ₐ are hydrogen. In certain embodiments, R₁, R₂, R₁ₐ and R₂ₐ are hydrogen and R₃ is —OH. In these instances, a compound of Formula (VII) can provide different compounds of Formula (VIII) depending on the choice to utilize only an enzyme that is at least 80% identical to PmOMT4 (SEQ ID NO: 5), or only an enzyme that is at least 80% identical to PmOMT1 (SEQ ID NO: 6), or both enzymes. In certain embodiments, R₁₃ is —CH₃ when a compound of Formula (VII) is reacted with an enzyme that is at least 80% identical to PmOMT4 (SEQ ID NO: 5). In certain embodiments, R₁₃ is hydrogen when a compound of Formula (VII) is reacted with an enzyme that is at least 80% identical to PmOMT1 (SEQ ID NO: 6). In certain embodiments, R₁₃ is —CH₃ when a compound of Formula (VII) is reacted with both an enzyme that is at least 80% identical to PmOMT4 (SEQ ID NO: 5) and an enzyme that is at least 80% identical to PmOMT1 (SEQ ID NO: 6). In certain embodiments, R₃ₐ is —OCH₃ when a compound of Formula (VII) is reacted with an enzyme that is at least 80% identical to PmOMT4 (SEQ ID NO: 5). In certain embodiments, R₃ₐ is —OH when a compound of Formula (VII) is reacted with an enzyme that is at least 80% identical to PmOMT1 (SEQ ID NO: 6). In certain embodiments, R₃ₐ is —OCH₃ when a compound of Formula (VII) is reacted with both an enzyme that is at least 80% identical to PmOMT4 (SEQ ID NO: 5) and an enzyme that is at least 80% identical to PmOMT1 (SEQ ID NO: 6). In certain embodiments, R₁₂ is —CH₃ and R₁₄ is hydrogen or R₁₂ is hydrogen and R₁₄ is —CH₃ when a compound of Formula (VII) is reacted with an enzyme that is at least 80% identical to PmOMT1 (SEQ ID NO: 6). In certain embodiments, R₁₂ and R₁₄ are hydrogen when a compound of Formula (VII) is reacted with an enzyme that is at least 80% identical to PmOMT4 (SEQ ID NO: 5). In certain embodiments, R₁₂ is —CH₃ and R₁₄ is hydrogen or R₁₂ is hydrogen and R₁₄ is —CH₃ when a compound of Formula (VII) is reacted with an enzyme that is at least 80% identical to PmOMT4 (SEQ ID NO: 5) and an enzyme that is at least 80% identical to PmOMT1 (SEQ ID NO: 6).

The methods to produce a compound of Formula (VIII) include reacting malonyl-CoA with a compound of Formula (VII) selected from the group consisting of:

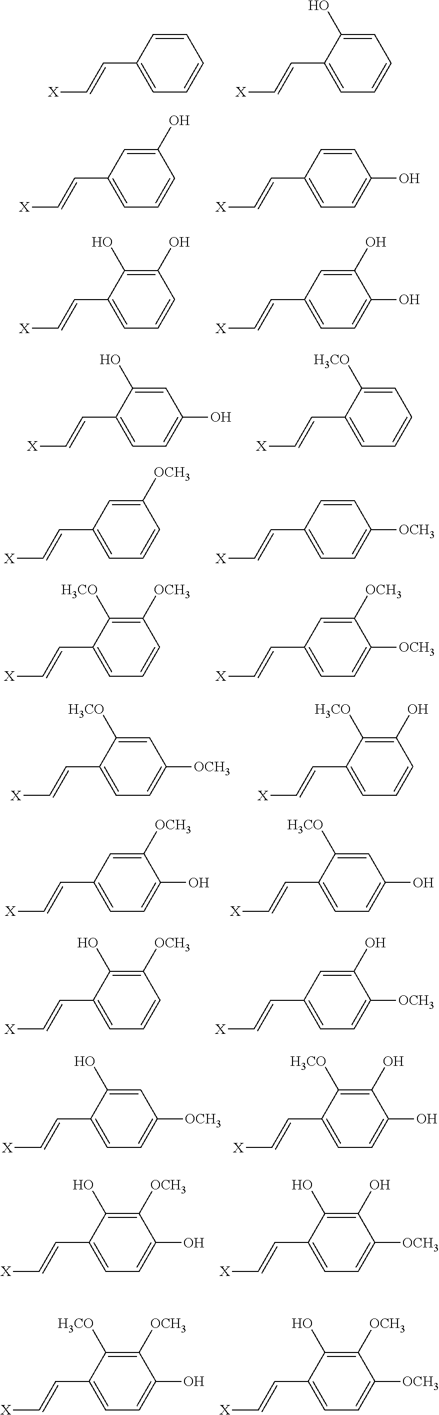

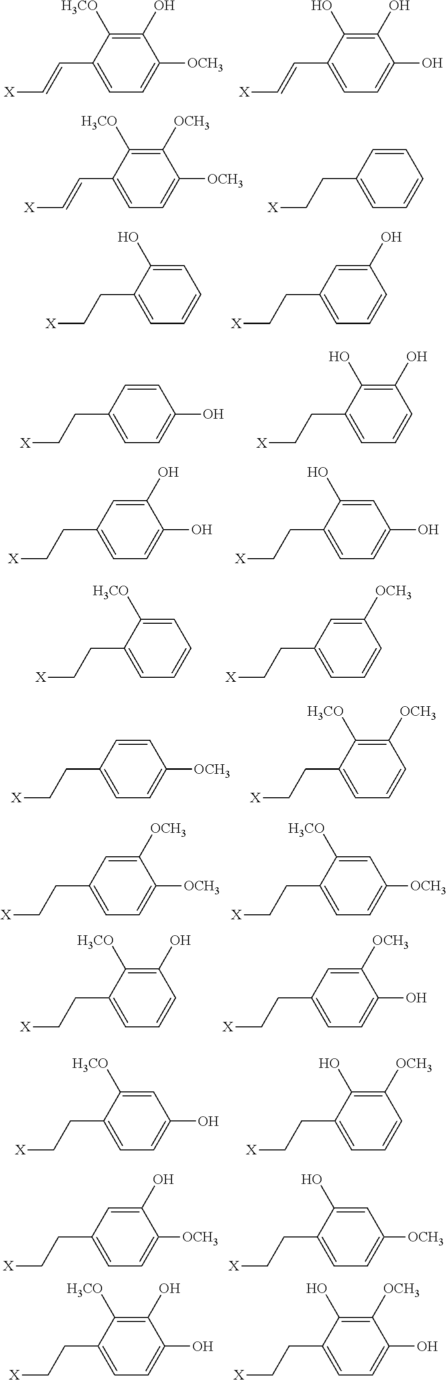

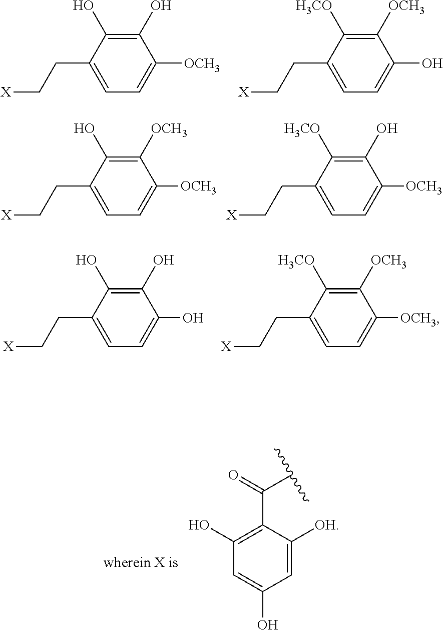

The methods to produce a compound of Formula (VIII) include culturing cells engineered to express an enzyme that is at least 80% identical to PmOMT4 (SEQ ID NO: 5). The methods to produce a compound of Formula (VIII) include culturing cells engineered to express an enzyme that is at least 80% identical to PmOMT1 (SEQ ID NO: 6). In certain embodiments, the enzyme is at least 80%, 85%, 90%, 95%, or 100% identical to PmOMT4 (SEQ ID NO: 5). In certain embodiments, the enzyme is at least 80%, 85%, 90%, 95%, or 100% identical to PmOMT1 (SEQ ID NO: 6). In certain embodiments, the enzyme is purified before reacting with a compound of Formula (VII). In certain embodiments, the enzyme is partially purified before reacting with a compound of Formula (VII).

In certain embodiments, the enzyme that is at least 80% identical to PmOMT4 (SEQ ID NO: 5) is a component in a fusion protein. In certain embodiments, the enzyme that is at least 80% identical to PmOMT1 (SEQ ID NO: 6) is a component in a fusion protein. A fusion protein may be created by joining two or more gene or gene segments that code for separate proteins. Translation of this fusion gene results in a single or multiple polypeptides with functional properties derived from each of the original proteins. A polyfunctional protein is a single protein that has at least two different activities, wherein that functionality is a native biological function or the result of an engineered enzyme fusion. Thus, a fusion protein may include multiple activities such as those described herein for the kavalactone or flavokavain pathway enzymes described herein (i.e., 4-coumarate-CoA ligase Pm4CL1 (at least 80% identical to SEQ ID NO: 1), styrylpyrone synthase PmSPS1 (at least 80% identical to SEQ ID NO: 2), PmSPS2 (at least 80% identical to SEQ ID NO: 3), PmCHS (at least 80% identical to SEQ ID NO: 4), methyltransferase PmOMT4 (at least 80% identical to SEQ ID NO: 5), methyltransferase PmOMT1 (at least 80% identical to SEQ ID NO: 6), cytochrome P450 enzyme PmMDB1 (at least 80% identical to SEQ ID NO: 7), and NADPH-dependent reductase PmRDCT10 (at least 80% identical to SEQ ID NO: 8)).

The enzyme that is at least 80% identical to PmOMT4 (SEQ ID NO: 5) is heterologous to the host cell. The enzyme that is at least 80% identical to PmOMT1 (SEQ ID NO: 6) is heterologous to the host cell. In certain embodiments, the enzyme that is at least 80% identical to PmOMT4 (SEQ ID NO: 5) is recombinantly produced. In certain embodiments, the enzyme that is at least 80% identical to PmOMT1 (SEQ ID NO: 6) is recombinantly produced. In certain embodiments, the enzyme that is at least 80% identical to PmOMT4 (SEQ ID NO: 5) is obtained from a wildtype organism. In certain embodiments, the enzyme that is at least 80% identical to PmOMT4 (SEQ ID NO: 5) is obtained from a mutant organism. In certain embodiments, the enzyme that is at least 80% identical to PmOMT4 (SEQ ID NO: 5) is obtained from a genetically-modified organism. In certain embodiments, the enzyme that is at least 80% identical to PmOMT1 (SEQ ID NO: 6) is obtained from a wildtype organism. In certain embodiments, the enzyme that is at least 80% identical to PmOMT1 (SEQ ID NO: 6) is obtained from a mutant organism. In certain embodiments, the enzyme that is at least 80% identical to PmOMT1 (SEQ ID NO: 6) is obtained from a genetically-modified organism. In certain embodiments, the organism is a non-human organism. In certain embodiments, the non-human organism is selected from group consisting of bacteria, yeast, and plant. In certain embodiments, the organism is a plant. In certain embodiments, the plant is Piper methysticum.

A nucleic acid encoding the enzyme may be introduced into the cell in a vector (e.g., plasmids, viral vectors, cosmids, and artificial chromosomes). In certain embodiments, the nucleic acid is cDNA derived from the amino acid sequence of the enzyme that is at least 80% identical to PmOMT4 (SEQ ID NO: 5). In certain embodiments, the nucleic acid is cDNA derived from the amino acid sequence of the enzyme that is at least 80% identical to PmOMT1 (SEQ ID NO: 6). In some embodiments multiple cDNAs comprising sequences complementary to different genes (e.g., 2, 3, 4, 5, or more genes) described herein (i.e., 4-coumarate-CoA ligase Pm4CL1 (at least 80% identical to SEQ ID NO: 1), styrylpyrone synthase PmSPS1 (at least 80% identical to SEQ ID NO: 2), PmSPS2 (at least 80% identical to SEQ ID NO: 3), PmCHS (at least 80% identical to SEQ ID NO: 4), methyltransferase PmOMT4 (at least 80% identical to SEQ ID NO: 5), methyltransferase PmOMT1 (at least 80% identical to SEQ ID NO: 6), cytochrome P450 enzyme PmMDB1 (at least 80% identical to SEQ ID NO: 7), and NADPH-dependent reductase PmRDCT10 (at least 80% identical to SEQ ID NO: 8)), are introduced into the same cell individually, or together, or as part of a single nucleic acid.

The host cells expressing the enzyme that is at least 80% identical to PmOMT4 (SEQ ID NO: 5) may be prokaryotic cells, such as bacterial cells (e.g., Escherichia coli cells), or eukaryotic cells, such as yeast cells or plant cells. The host cells expressing the enzyme that is at least 80% identical to PmOMT1 (SEQ ID NO: 6) may be prokaryotic cells, such as bacterial cells (e.g., Escherichia coli cells), or eukaryotic cells, such as yeast cells or plant cells. In certain embodiments, the host cell is capable of expressing two or more kavalactone or flavokavain pathway enzymes described herein. In certain embodiments, the host cell is a bacteria cell and is a wildtype, mutant, recombinant, or genetically engineered form of Escherichia coli. In certain embodiments, the host cell is a yeast cell and is a wildtype, mutant, recombinant, or genetically engineered form of Saccharomyces cerevisiae. In certain embodiments, the host cell is a plant cell and is a wildtype, mutant, recombinant, or genetically engineered form of Nicotiana benthamiana.

In certain embodiments, the method for producing a compound of Formula (VIII) utilizes a compound of Formula (I), or a salt thereof, as the starting material and comprises the steps: condensing a compound of Formula (I), or a salt thereof, with coenzyme A (CoA) using an enzyme that is at least 80% identical to Pm4CL1 (SEQ ID NO: 1) to produce a compound of Formula (II); reacting a compound of Formula (II), or a salt thereof, with malonyl-CoA using an enzyme that is at least 80% identical to PmCHS (SEQ ID NO: 4) to produce a compound of Formula (VII); and methylating a compound of Formula (VII), or a salt thereof, with S-adenosylmethionine using an enzyme that at least 80% identical to PmOMT4 (SEQ ID NO: 5).

In certain embodiments, the method for producing a compound of Formula (VIII) utilizes a compound of Formula (I), or a salt thereof, as the starting material and comprises the steps: condensing a compound of Formula (I), or a salt thereof, with coenzyme A (CoA) using an enzyme that is at least 80% identical to Pm4CL1 (SEQ ID NO: 1) to produce a compound of Formula (II); reacting a compound of Formula (II), or a salt thereof, with malonyl-CoA using an enzyme that is at least 80% identical to PmCHS (SEQ ID NO: 4) to produce a compound of Formula (VII); and methylating a compound of Formula (VII), or a salt thereof, with S-adenosylmethionine using an enzyme that at least 80% identical to PmOMT1 (SEQ ID NO: 6).

### In Vitro Reactions

In vitro reactions are utilized in the present disclosure. In certain embodiments, the reactions use water as a solvent. In certain embodiments, the reaction is performed at room temperature. In certain embodiments, the reaction is performed for 10 minutes to 24 hours. In certain embodiments, the reaction is performed for 2 hours.

The components of the reactions may include one of more of the following: buffer; MgCl₂; ATP; CoA; malonyl-CoA; a compound of Formula (I), Formula (II), Formula (III), Formula (IV), or Formula (VII); S-adenosylmethionine; NADPH; and an enzyme described herein. In certain embodiments, the buffer is potassium phosphate of pH=7.6. In certain embodiments, the concentration of the buffer is 50 mM. In certain embodiments, the concentration of the MgCl₂ is 2.5 mM. In certain embodiments, the concentration of the ATP is 3 mM. In certain embodiments, the concentration of CoA is 2 mM. In certain embodiments, the concentration of malonyl CoA is 3 mM. In certain embodiments the concentration of the compound described herein is 0.5 mM. In certain embodiments, the concentration of S-adenosylmethionine is 2 mM. In certain embodiments, the concentration of NADPH is 6 mM. In certain embodiments, the concentration of an enzyme described herein is 10 μg/ml final concentration of each enzyme used in the reaction.

### EXAMPLES

In order that the invention described herein may be more fully understood, the following examples are set forth. The examples described in this application are offered to illustrate the methods, compositions, and systems provided herein and are not to be construed in any way as limiting their scope.

### Example 1. Activity of Pm4CL1, PmSPS1, PmSPS2, and PmCHS

Using purified recombinant enzymes expressed in Escherichia coli, we have shown the activity of PmSPS1, PmSPS2, and PmCHS in vitro (FIG. 4). This assay used p-coumaric acid as a substrate and utilized the purified 4-coumarate-CoA ligase Pm4CL1 to produce p-coumaroyl-CoA.

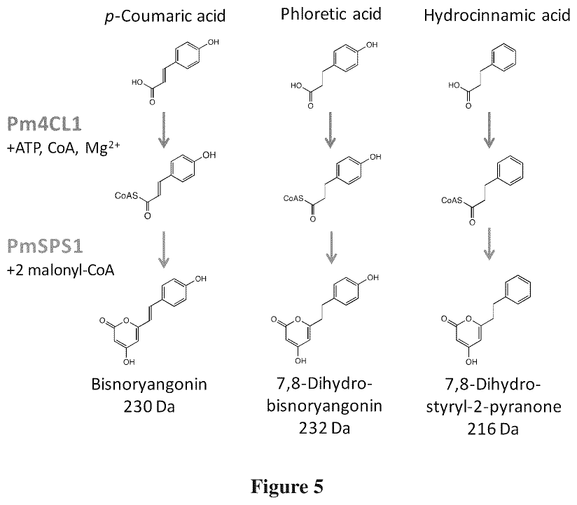

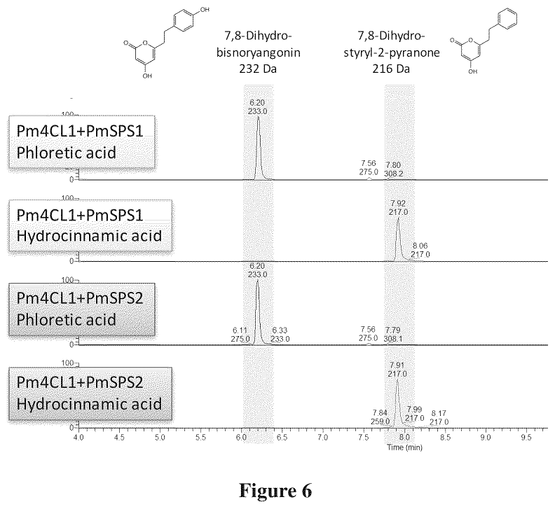

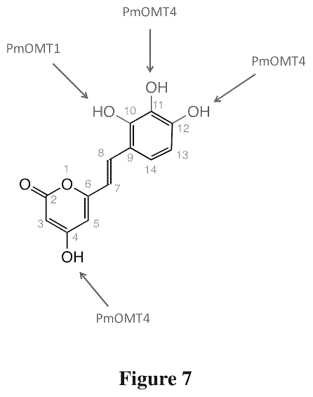

The PmSPS1 and PmSPS2 enzymes can also utilize substrates derived from cinnamic acid variants phloretic acid and hydrocinnamic (phenylpropanoic) acid, which include a single bond instead of the double bond present in cinnamic acid (FIG. 5). This results in a 6-styryl-4-hydroxy-2-pyrone backbone with a single bond at C₇-C₈ position, which can be used to produce reduced kavalactones, such as 7,8-dihydrokavain or 7,8-dihydroyangonin. This activity was verified using in-vitro enzyme assays monitored by LC-MS (FIG. 6).

### Example 2. Activity of PmOMT4 and PmOMT1

Two methyltransferases, PmOMT4 and PmOMT1, add methyl groups to hydroxyl groups at various positions of the 6-styryl-4-hydroxy-2-pyrone backbone. PmOMT4 is the key methyltransferase that adds a methyl group to the 4-position, as seen in all kavalactones. In addition, PmOMT4 can methylate the C₁₁ and C₁₂ positions (if hydroxyl groups are present there), as found, for example, in 11-methoxyyangonin (FIG. 7). On the other hand, PmOMT1 adds a methyl group to the C₁₀ position, as found, for example, in 10-methoxyyangonin (FIG. 7).

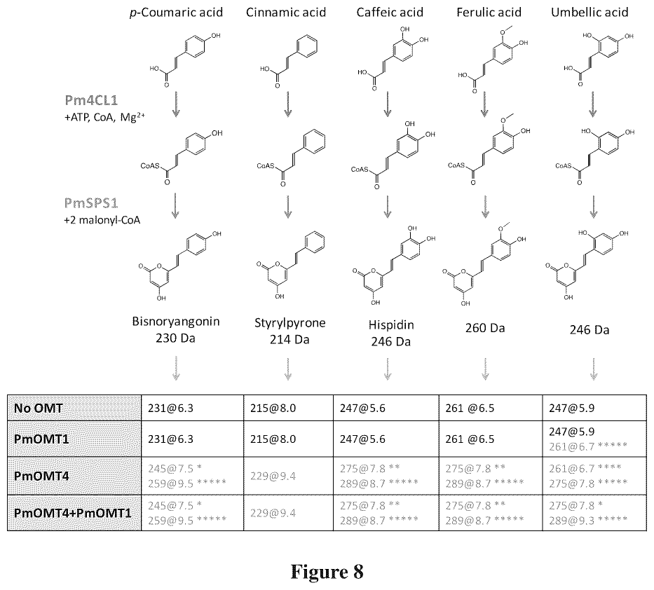

The target hydroxyl sites of PmOMT1 and PmOMT4 were determined by coupled enzyme assays using different starting substrates (variants of cinnamic acid with different hydroxy modifications on the aromatic ring). The enzyme assay utilized Pm4CL1 and PmSPS1 to produce the 6-styryl-4-hydroxy-2-pyrone backbone, and increase in mass after adding the methyltransferases was monitored by LC-MS (FIG. 8).

### Example 3. Activity of PmRDCT10

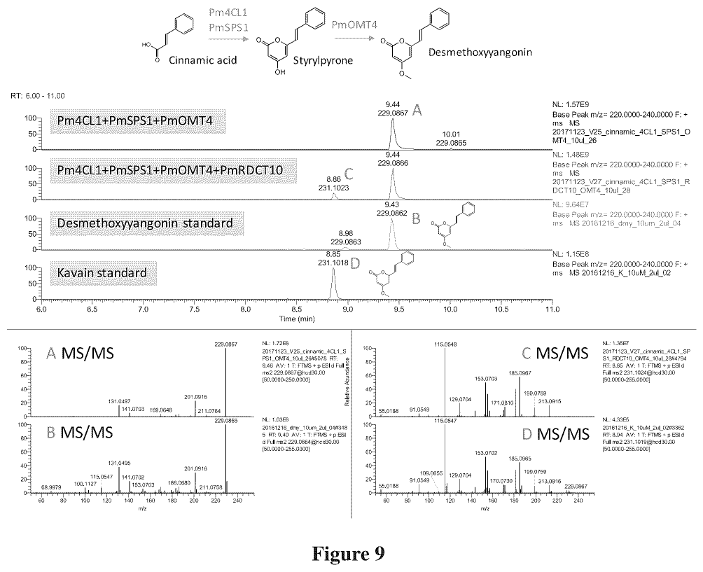

The C₅-C₆ double bond in kavalactones can be reduced into a single bond by an NADPH-dependent reductase PmRDCT10, as demonstrated by another in vitro enzyme assay (FIG. 9). This reaction is essential to produce reduced kavalactones such as kavain or methysticin.

### Example 4. Activity of PmMDB1

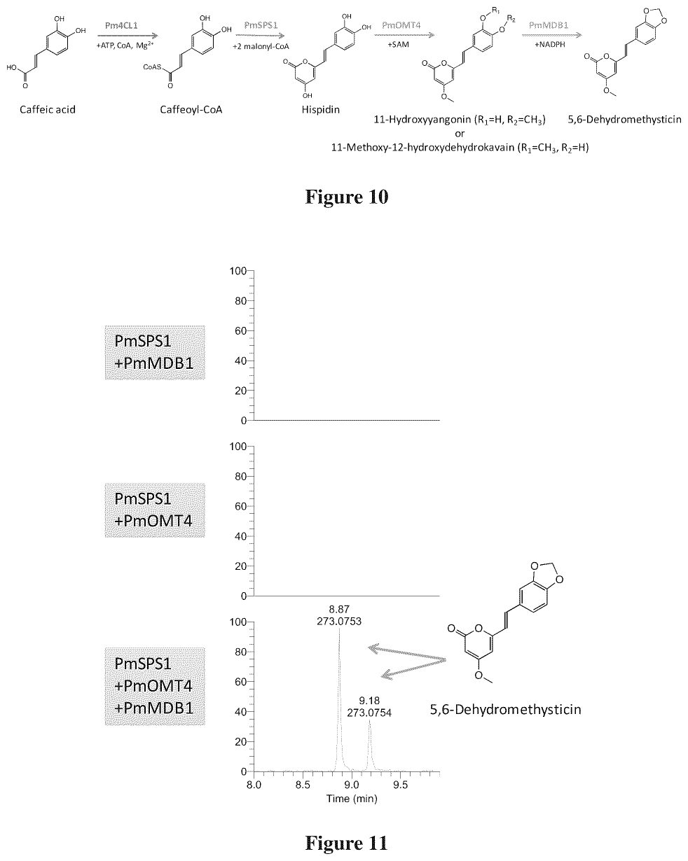

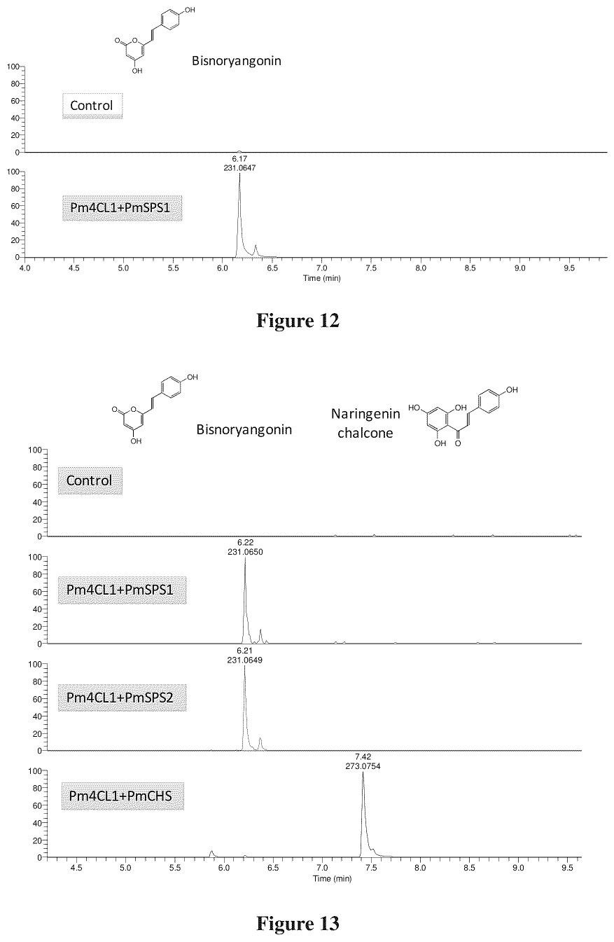

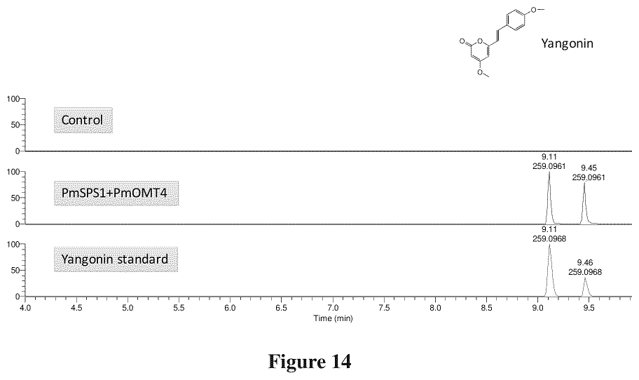

The methylenedioxy bridge found at the C₁₁-C₁₂ position in several kavalactones (methysticin, dihydromethysticin, or dehydromethysticin) is formed by a cytochrome P450 enzyme PmMDB1, which belongs to the CYP719 family (FIG. 10).

The activity of PmMDB1 was confirmed by agrobacterium-mediated heterologous expression in Nicotiana benthamiana (FIG. 11). This assay utilized the native Nicotiana 4CL.

Example 5. Enzyme Amino Acid Sequences
Pm4CL1 (SEQ ID NO: 1):
MKMVVDTIATDRCVYRSKLPDIEIKNDMSLHNYCFQNIGAYRDNPCLINGSTGEVYTYGEVETTARRVAAGLHRMGV
QQREVIMILLPNSPEFVFAFLGASFRGAMSTTANPFYTPQEIAKQVKASGAKLIVTMSAYVDKVRDLAEERGVKVVC
VDAPPPGCSHFSELSGADESELPEVDIDPDDVVALPYSSGTTGLPKGVMLTHRSQVTSVAQQVDGENPNLYFRPDDV
LLCVLPLFHIYSLNSVLFCGLRVGAAILIMQKFEITALMELVQKYKVTIAPIVPPIVLAIAKSPLVDKYDLSSIRTV
MSGAAPMGKELEDAVRAKLPNAKLGQGYGMTEAGPVLSMCLAFAKEPFEIKSGSCGTVVRNAQLKIVDPETGAYLPR
NQPGEICIRGSQIMKGYLNDAAATQRTIDKEGWLHTGDIGYVDDDEELFIVDRLKEIIKYKGFQVAPAELEAILITH
PNIADAAVVPMKDEAAGEVPVAFVVTSNGSVISEDEIKQFISKQVVFYKRINRVFFVDSIPKAPSGKILRKDLRGRL
AAGIPK
PmSPS1 (SEQ ID NO: 2):
MSKTVEDRAAQRAKGPATVLAIGTATPANVVYQTDYPDYYFRVTKSEHMTKLKNKFQRMCDRSTIKKRYMVLTEELL
EKNLSLCTYMEPSLDARQDILVPEVPKLGKEAADEAIAEWGRPKSEITHLIFCTTCGVDMPGADYQLTKLLGLRSSV
RRTMLYQQGCFGGGTVLRLAKDLAENNAGARVLVVCSEITTAVNFRGPSDTHLDLLVGLALFGDGAAAVIVGADPDP
TLERPLFQIVSGAQTILPDSEGAINGHLREVGLTIRLLKDVPGLVSMNIEKCLMEAFAPMGIHDWNSIFWIAHPGGP
TILDQVEAKLGLKEEKLKSTRAVLREYGNMSSACVLFILDEVRKRSMEEGKTTTGEGFDWGVLFGFGPGFTVETVVL
HSMPIPKADEGR
PmSPS2 (SEQ ID NO: 3):
MSKMVEEHWAAQRARGPATVLAIGTANPPNVLYQADYPDFYFRVTKSEHMTQLKEKFKRICDKSAIRKRHLHLTEEL
LEKNPNICAHMAPSLDARQDIAVVEVPKLAKEAATKAIKEWGRPKSDITHLIFCTTCGVDMPGADYQLTTLLGLRPT
VRRTMLYQQGCFAGGTVLRHAKDFAENNRGARVLAVCSEFTVMNFSGPSEAHLDSMVGMALFGDGASAVIVGADPDF
AIERPLFQLVSTTQTIVPDSDGAIKCHLKEVGLTLHLVKNVPDLISNNMDKILEEAFAPLGIRDWNSIFWTAHPGGA
AILDQLEAKLGLNKEKLKTTRTVLREYGNMSSACVCFVLDEMRRSSLEEGKTTSGEGLEWGILLGFGPGLTVETVVL
RSVPISTAN
PmCHS (SEQ ID NO: 4):
MSKTVEEIWAAQRARGPATVLAIGTAAPANVVYQADYPDYYFRITKSEHMTELKEKFRRMCDKSMITKRHMHLSEEL
LKNNPDICAYMAPSLDARQDMVVVEVPKLGKEAAAKAIKEWGRPKSAITHLIFCTTSGVDMPGADFQLTKLLGLCPS
VRRTMLYQQGCFAGGTVLRLAKDLAENNAGARVLVVCSEITAVTFRGPSETHLDSMVGQALFGDGASAIIVGADPDP
VIERPLFQIVSAAQTILPDSDGAIDGHLREVGLTFHLLKDVPGLISKNIEKSLKEAFAPLGIDDWNSTWIVHPGGPA
ILDQVEAKLRLKVEKLKTTRTVLSEYGNMSSACVLFILDEMRRNSMEEGKATTGEGLHWGVLFGFGPGLTVETVVLH
SLPIAEAN
PmOMT4 (SEQ ID NO: 5):
MEQAVFKDQSPSRDDIDEELFQSALYLSTAVVTVPAAIMAANDLDVLQIIAKAGPGAHLSPTEIVSHLPTRNPNAAA
ALHRILRVLASHSILECSSRCEGEAKYGLRPVCKFFLNDKDGVSLNAMPSFVQSRVFIDSWQYMKDAVLEGVVPFEK
AYGMPFYQFQAVNTKFKETFAKAMAAHSTLVVKKMLDTYNGFEGLTELMDVAGGTGSTLNLIVSKYPQIKGTNFDLK
HVIEAAPNYPGVKHLSGDMFDSIPSAKNIIMKWILHNWSDEHCVKLLKNCYTSLPEFGKLIVVDSIVGEDVDAGLTT
TNVFGCDFTMLTFFPNAKERTREEFQDLAKASGFSTFKPICCAYGVWVMEFHK
PmOMT1 (SEQ ID NO: 6):
MNDQELHGYSQNAQPQLWNLLLSFINSMSLKCAVELGIPDIIHSHAQTPINTITDLAASIPIPPNKTSQFRRLMRLL
VHSNVFSVHKREDGDEGFLLTPMSRILVTSNDNNGGNLSPFVSMMVDPSLVSPWHFLGQWLKGNDTQGTPFRMCHGE
EMWDWANKYPDFNKKFNMAMVCDSQYLMKIIVKKCATAFEGKRSLIDVGGGTGGAARSIAEAFPDIQEVSVLDLPHV
VAGLPNDSRVKFVGGDMFHTIPPADVVLLKAIFHGWNDEECIKILKNCKKAIPSKEEGGKVMILDMVVNSAPGDHMI
TEDQYFMDLMMITYARGLERDENEWKKLFKDAGFTSYKITHGLGTSSLIELYP
PmMDB1 (SEQ ID NO: 7):
MEQAQWVDPTLLPAFVGIIFFFLGMFFGRSSLGAGKGAAPRSTSSTEWPDGPPKLPIIGNLHQLNKGGELVHHNLAK
LAQSYDRAMTIWVGSWGPMIVVSDADLAWEVLVTKSPDFAGRVLSKLSHLFNANYNTVVAYDAGPQWQSLRRGLQHG
PLGPAHVSAQARFHEEDMKLLVSDMMRAAQKGGSNGVVEPLAYVRRATIRFLSRLCFGEAFNDEAFVEGMDEAVEET
IGATGHARILDAFYFTRHLPIIRRSFIDTVNAKKKIESLVRPLLSRPAPPGSYLHFLLSTDAPENMIIFRIFEVYLL
GVDSTASTTTWALAFLVSNQQAQEKLHNELAQYCASQNNQIIKADDVGKLSYLLGVVKETMRMKPIAPLAVPHKTLK
ETMLDGKRVAAGTTVVVNLYAVHYNPKLWPEPEQFRPERFVVGASGGNGGGSSEYMLQSYLPFGGGMRSCAGMEVGK
LQVAMVVANLVMAFKWLPEEEGKMPDLAEDMTFVLMMKKPLAAKIVPRA
PmRDCT10 (SEQ ID NO: 8):
METERKSRICVTGAGGFVASWVVKLFLSKGYLVHGTVRDLGEEKTAHLRKLEGAYHNLQLFKADLLDYESLLGAITG
CDGVLHVATPVPSSKTAYSGTELVKTAVNGTLNVLRACTEAKVKKVIYVSSTAAVLVNPNLPKDKIPDEDCWTDEEY
CRTTPFFLNWYCIAKTAAEKNALEYGDKEGINVISICPSYIFGPMLQPTINSSNLELLRLMKGDDESIENKFLLMVD
VRDVAEAILLLYEKQETSGRYISSPHGMRQSNLVEKLESLQPGYNYHKNFVDIKPSWTMISSEKLKKLGWKPRPLED
TISETVLCFEEHGLLENE
Example 6. cDNA Sequences
cDNA Sequence Encoding for Pm4CL1 (SEQ ID NO: 9)
ATGAAGATGGTAGTAGACACTATTGCTACTGATCGATGTGTATACCGGTCTAAGCTGCCGGACATTGAGATCAAGAA
CGACATGTCGTTGCACAATTATTGTTTCCAGAACATTGGTGCTTACCGGGACAATCCTTGTCTCATCAATGGCAGCA
CCGGCGAGGTGTACACGTACGGCGAGGTGGAGACGACGGCGAGGAGGGTGGCCGCCGGGCTGCACCGGATGGGGGTG
CAGCAGCGGGAGGTGATCATGATCCTCCTCCCCAACTCGCCGGAGTTCGTCTTCGCCTTCCTCGGCGCCTCCTTCCG
CGGGGCCATGTCCACCACCGCCAACCCCTTCTACACGCCGCAGGAGATCGCCAAGCAGGTCAAGGCCTCCGGCGCGA
AGCTCATCGTCACCATGTCCGCCTACGTCGACAAGGTCAGGGACCTGGCCGAGGAGCGCGGCGTCAAAGTGGTGTGC
GTCGACGCGCCGCCCCCGGGGTGCTCCCACTTCTCCGAGCTGTCCGGCGCCGACGAGTCGGAGCTGCCCGAGGTGGA
TATTGACCCCGACGACGTGGTGGCGCTGCCATACTCCTCCGGCACCACCGGCCTCCCTAAAGGAGTGATGCTCACAC
ACCGCAGCCAGGTGACGAGCGTTGCCCAGCAAGTCGACGGCGAGAACCCGAATCTATACTTCCGGCCAGACGACGTC
CTGCTCTGCGTTCTTCCCCTCTTCCACATCTACTCCCTCAACTCGGTGCTCTTCTGCGGCCTGCGCGTCGGGGCGGC
GATCCTCATCATGCAGAAGTTCGAGATCACGGCGCTGATGGAGCTGGTGCAGAAGTACAAGGTGACCATTGCGCCCA
TCGTTCCGCCCATCGTTCTTGCCATCGCCAAGAGCCCGCTCGTCGACAAGTACGACTTGTCGTCCATTCGGACGGTG
ATGTCCGGCGCCGCCCCGATGGGGAAGGAGCTCGAAGACGCCGTCCGGGCCAAGCTTCCCAACGCCAAGCTCGGCCA
GGGCTATGGGATGACGGAGGCAGGGCCAGTGCTGTCCATGTGTTTGGCCTTCGCCAAGGAGCCCTTCGAGATCAAGT
CTGGTTCTTGCGGCACCGTGGTCAGGAACGCCCAGCTCAAGATCGTCGACCCAGAAACCGGTGCCTACCTGCCCAGA
AACCAACCCGGCGAAATTTGCATCCGAGGCTCCCAAATCATGAAAGGGTATCTTAATGACGCGGCGGCTACGCAGAG
GACGATCGACAAGGAAGGGTGGCTGCACACCGGCGACATTGGCTATGTCGACGACGACGAGGAGCTCTTCATTGTCG
ATAGGTTGAAGGAGATCATTAAGTACAAGGGCTTCCAAGTCGCCCCTGCCGAGCTCGAAGCCATTCTCATTACTCAC
CCTAACATTGCTGATGCCGCTGTTGTCCCGATGAAAGATGAGGCAGCAGGGGAAGTGCCAGTGGCATTTGTGGTGAC
CTCCAATGGATCAGTCATCAGTGAGGATGAGATCAAGCAGTTCATTAGCAAGCAGGTGGTGTTCTACAAGCGAATCA
ATCGAGTCTTTTTCGTTGATTCAATTCCTAAAGCACCCTCTGGGAAGATTTTGAGGAAGGATTTGAGGGGAAGATTG
GCAGCTGGTATACCCAAGTAG
cDNA Sequence Encoding for PmSPS1 (SEQ ID NO: 10)
ATGTCGAAGACGGTGGAGGATCGGGCAGCGCAGCGGGCAAAGGGGCCGGCAACAGTGCTGGCCATCGGCACGGCTAC
GCCGGCCAATGTGGTGTACCAGACCGATTATCCGGACTACTACTTCAGGGTCACCAAGAGCGAGCATATGACCAAAC
TCAAGAACAAGTTTCAACGCATGTGCGACAGGTCGACGATAAAGAAGAGGTACATGGTTTTGACAGAGGAGCTGCTA
GAGAAGAATCTGAGTTTGTGCACCTACATGGAACCCTCCCTCGACGCCCGCCAAGACATTCTCGTGCCGGAGGTCCC
CAAGCTCGGCAAGGAGGCCGCCGACGAGGCCATCGCCGAATGGGGACGCCCCAAGTCGGAAATCACCCACCTCATCT
TTTGCACTACCTGCGGCGTCGACATGCCCGGCGCCGACTACCAGCTCACCAAGCTCCTCGGTCTCCGCTCCTCCGTC
CGTCGCACCATGCTCTATCAGCAGGGATGCTTTGGCGGAGGCACCGTTCTCCGCCTCGCCAAGGACCTCGCCGAGAA
CAACGCTGGTGCCCGCGTCCTCGTCGTCTGCTCCGAGATCACCACTGCCGTCAACTTCCGAGGGCCTTCCGACACCC
ACCTCGACTTATTGGTCGGCTTAGCCCTGTTCGGCGACGGTGCGGCCGCGGTCATAGTCGGTGCGGATCCAGATCCT
ACCCTCGAGCGGCCGCTCTTTCAAATCGTATCTGGAGCACAGACGATTCTACCGGACTCGGAGGGGGCCATCAACGG
CCATCTCCGGGAGGTGGGGCTAACCATCCGCCTACTCAAGGACGTACCTGGGCTTGTGTCGATGAACATTGAGAAGT
GCCTCATGGAGGCGTTTGCACCGATGGGCATCCACGACTGGAACTCCATCTTTTGGATAGCCCATCCCGGGGGGCCC
ACCATACTAGACCAAGTGGAGGCCAAGCTGGGTCTAAAGGAGGAGAAGCTCAAGTCGACGAGGGCTGTTCTGAGGGA
GTATGGCAACATGTCTAGCGCCTGTGTCTTGTTCATACTGGACGAGGTAAGGAAGAGGAGCATGGAGGAGGGGAAGA
CGACAACCGGTGAGGGGTTCGATTGGGGAGTTCTATTCGGCTTTGGGCCTGGCTTCACAGTGGAGACCGTCGTCTTG
CACAGCATGCCCATCCCCAAAGCCGATGAAGGCAGATAA
cDNA Sequence Encoding for PmSPS2 (SEQ ID NO: 11)
ATGTCGAAGATGGTGGAGGAGCATTGGGCAGCGCAGCGGGCGAGGGGACCGGCGACAGTGCTGGCCATCGGCACTGC
AAATCCTCCCAATGTGTTGTACCAGGCAGATTATCCCGACTTCTACTTTAGGGTCACCAAGAGTGAGCACATGACCC
AGCTAAAGGAGAAGTTTAAACGTATATGTGATAAGTCAGCAATAAGAAAGCGCCACCTCCATCTAACCGAGGAGCTG
CTGGAGAAGAACCCTAACATATGTGCACACATGGCCCCCTCCCTCGACGCCCGGCAAGACATTGCGGTGGTGGAGGT
CCCCAAGCTAGCCAAAGAAGCTGCAACCAAGGCCATCAAGGAGTGGGGGCGACCCAAGTCCGACATCACCCACCTCA
TCTTCTGCACCACCTGCGGCGTGGACATGCCCGGCGCCGACTACCAACTCACCACGCTCCTCGGCCTCCGCCCCACG
GTCCGCCGCACCATGCTCTACCAACAGGGCTGCTTCGCCGGCGGCACAGTCCTTCGCCATGCCAAGGACTTCGCCGA
GAACAATAGGGGTGCTCGTGTCCTCGCCGTCTGCTCGGAGTTCACCGTCATGAACTTCAGCGGACCGTCGGAGGCCC
ACTTAGACAGCATGGTCGGTATGGCGCTGTTCGGTGATGGCGCCTCGGCTGTCATCGTCGGCGCCGATCCTGACTTT
GCCATTGAACGACCGCTCTTTCAACTGGTTTCTACAACACAAACTATTGTCCCGGACTCGGACGGAGCCATCAAGTG
CCATCTCAAGGAGGTGGGCCTAACCCTGCATCTCGTTAAGAATGTACCAGATCTCATATCAAATAACATGGACAAGA
TCCTCGAAGAGGCATTTGCACCATTGGGCATCAGAGATTGGAACTCAATCTTTTGGACAGCTCATCCAGGTGGAGCA
GCCATACTCGACCAGTTGGAGGCCAAGCTCGGTCTGAACAAGGAGAAGCTCAAGACTACAAGAACAGTTCTGAGGGA
GTATGGAAACATGTCCAGCGCCTGTGTTTGTTTCGTCCTGGACGAGATGAGGAGAAGTAGCTTGGAGGAGGGGAAGA
CAACGTCCGGGGAAGGGTTGGAATGGGGAATTCTGCTAGGGTTTGGGCCTGGGTTGACAGTGGAGACAGTCGTCTTG
CGTAGCGTACCCATCTCGACAGCCAATTAA
cDNA Sequence Encoding for PmCHS (SEQ ID NO: 12)
ATGTCGAAGACCGTAGAGGAGATTTGGGCGGCGCAGCGGGCGAGGGGACCAGCCACGGTGCTGGCCATCGGCACTGC
TGCGCCGGCCAATGTGGTGTACCAGGCCGATTATCCGGACTACTACTTTAGGATCACCAAGAGCGAGCACATGACAG
AGCTCAAGGAGAAGTTCCGACGAATGTGTGACAAGTCGATGATAACGAAGCGGCACATGCACTTGTCGGAGGAGCTG
TTGAAAAACAACCCTGACATCTGTGCCTACATGGCCCCTTCCCTCGACGCCCGCCAAGATATGGTCGTGGTGGAGGT
ACCCAAGCTCGGCAAGGAGGCGGCCGCCAAGGCCATCAAGGAATGGGGCCGCCCAAAGTCGGCCATCACCCACCTCA
TCTTCTGCACCACCTCCGGCGTCGACATGCCCGGCGCCGATTTCCAGCTCACCAAGCTACTCGGCCTCTGCCCCTCC
GTTCGCCGCACCATGCTCTACCAGCAGGGCTGCTTCGCCGGCGGTACGGTTCTCCGCCTTGCCAAGGACCTCGCCGA
GAACAATGCGGGCGCGAGGGTCCTCGTCGTCTGCTCCGAGATCACCGCCGTCACCTTCCGCGGCCCCTCGGAGACTC
ACCTCGATAGCATGGTCGGCCAGGCCCTGTTCGGTGATGGTGCCTCTGCCATCATCGTCGGTGCCGACCCCGACCCC
GTCATAGAAAGGCCACTCTTTCAAATTGTATCTGCGGCTCAGACCATCCTTCCCGACTCGGATGGGGCAATAGACGG
CCATCTCCGAGAAGTGGGTCTAACCTTCCACCTCCTCAAGGACGTACCTGGGCTCATCTCAAAGAACATCGAGAAGA
GCCTAAAGGAGGCGTTTGCACCGCTGGGCATCGACGACTGGAACTCGATATTTTGGATTGTTCATCCAGGCGGGCCG
GCCATTCTAGACCAGGTGGAGGCGAAGCTGCGTCTGAAAGTGGAGAAGCTGAAGACAACGAGAACAGTTTTGAGTGA
GTACGGGAATATGTCGAGCGCTTGCGTGTTGTTCATACTTGACGAGATGAGGAGGAACAGCATGGAAGAAGGGAAGG
CGACGACCGGTGAAGGGTTACATTGGGGAGTTTTGTTTGGTTTTGGGCCGGGCTTGACAGTGGAGACGGTCGTCTTG
CATAGTTTGCCCATCGCCGAGGCCAACTAA
cDNA Sequence Encoding for PmOMT4 (SEQ ID NO: 13)
ATGGAGCAAGCTGTGTTCAAAGACCAATCCCCAAGCAGGGATGATATTGATGAAGAGCTCTTTCAATCTGCTCTATA
TCTTAGCACTGCGGTTGTCACCGTGCCGGCGGCAATCATGGCTGCAAATGACCTTGACGTGCTGCAGATAATTGCCA
AAGCTGGCCCAGGTGCTCACCTATCTCCGACAGAGATTGTCAGCCACCTTCCCACCCGTAACCCTAATGCCGCGGCG
GCGCTTCACCGGATACTCCGAGTACTAGCCAGCCACTCCATCCTTGAATGCTCGTCGAGATGCGAGGGCGAGGCAAA
ATATGGATTAAGGCCGGTGTGCAAGTTCTTTCTCAATGATAAGGATGGTGTCTCCTTGAATGCCATGCCATCCTTCG
TTCAAAGTAGAGTTTTTATAGATAGCTGGCAATATATGAAAGATGCTGTTCTTGAGGGGGTAGTCCCCTTTGAGAAA
GCCTATGGTATGCCTTTTTATCAGTTTCAAGCAGTGAACACCAAATTCAAAGAAACCTTCGCCAAAGCCATGGCTGC
TCACTCAACTTTGGTAGTAAAAAAGATGCTTGACACATACAATGGGTTTGAGGGACTCACTGAGTTGATGGATGTTG
CTGGTGGAACCGGTTCCACCCTCAACCTCATTGTCTCCAAATACCCACAAATCAAAGGCACAAACTTTGATCTCAAA
CATGTCATTGAGGCCGCACCAAACTACCCTGGGGTGAAGCATTTGAGTGGGGACATGTTTGATAGCATTCCAAGTGC
AAAGAACATTATTATGAAGTGGATACTACATAATTGGAGCGACGAGCACTGTGTAAAACTCCTCAAGAACTGCTACA
CTTCCTTACCAGAATTTGGGAAGTTGATTGTGGTTGATTCCATTGTGGGTGAGGATGTTGATGCTGGTTTGACGACA
ACAAATGTCTTTGGATGCGACTTCACAATGCTAACTTTCTTCCCCAATGCAAAAGAGAGGACCCGTGAAGAATTCCA
AGACCTGGCCAAAGCTAGTGGCTTCTCAACGTTCAAACCGATCTGCTGCGCCTATGGCGTGTGGGTTATGGAATTTC
ACAAATAA
cDNA Sequence Encoding for PmOMT1 (SEQ ID NO: 14)
ATGAATGATCAAGAGTTGCATGGATACTCACAAAATGCTCAACCTCAGCTATGGAACCTCCTGTTGAGCTTCATAAA
TTCCATGTCCCTTAAGTGTGCAGTGGAGTTGGGCATCCCCGATATAATACATAGCCATGCCCAAACACCAATCAACA
TAACCGACCTTGCTGCCTCCATACCCATTCCCCCAAACAAAACAAGCCAATTCCGCCGACTCATGCGCCTCCTGGTT
CACTCCAACGTCTTTTCCGTCCATAAACGTGAGGATGGTGATGAGGGGTTCCTCCTAACTCCTATGTCCAGGATCCT
TGTCACGTCGAACGACAATAATGGAGGTAACTTGTCACCCTTTGTTTCCATGATGGTTGATCCGTCCCTGGTGTCTC
CATGGCACTTCCTTGGTCAATGGCTCAAAGGCAATGACACCCAAGGCACACCATTTCGCATGTGCCATGGTGAAGAA
ATGTGGGACTGGGCCAACAAGTACCCGGACTTCAACAAGAAGTTCAACATGGCGATGGTCTGTGACAGCCAGTATTT
AATGAAAATTATTGTGAAGAAGTGCGCCACTGCCTTTGAAGGCAAGAGGTCCCTGATTGACGTCGGTGGCGGGACTG
GTGGCGCCGCACGGTCTATTGCCGAAGCATTTCCAGACATACAGGAGGTGTCTGTATTGGATCTTCCTCATGTGGTT
GCAGGTTTGCCCAATGACTCGAGGGTGAAGTTTGTTGGAGGAGACATGTTCCACACCATCCCTCCCGCTGATGTTGT
CTTATTGAAGGCGATTTTTCATGGTTGGAATGATGAGGAGTGCATCAAGATATTGAAGAACTGCAAGAAGGCAATTC
CAAGCAAGGAAGAGGGAGGCAAGGTGATGATATTGGACATGGTGGTCAATTCCGCCCCGGGTGACCATATGATTACA
GAAGATCAATATTTTATGGATTTGATGATGATAACCTACGCAAGAGGATTGGAGAGAGACGAGAATGAATGGAAGAA
GCTGTTTAAAGATGCAGGTTTCACATCGTACAAGATCACCCACGGGCTTGGAACGAGTTCGCTTATCGAGCTCTACC
CTTAG
cDNA Sequence Encoding for PmMDB1 (SEQ ID NO: 15)
ATGGAGCAAGCTCAATGGGTCGACCCAACTCTGCTCCCTGCATTTGTGGGCATCATCTTCTTCTTCCTTGGCATGTT
CTTTGGAAGGAGTTCTTTGGGAGCTGGGAAGGGTGCAGCGCCTAGAAGCACCAGTTCTACCGAGTGGCCAGACGGCC
CTCCAAAGCTGCCCATCATCGGCAACCTGCACCAGCTCAACAAAGGCGGGGAGCTGGTCCACCACAACCTCGCCAAG
CTCGCCCAGTCCTACGACCGCGCCATGACCATCTGGGTCGGCAGCTGGGGCCCCATGATCGTCGTCAGCGACGCCGA
CCTTGCATGGGAGGTCCTCGTCACCAAGTCGCCGGACTTCGCCGGCCGGGTGCTCTCCAAGCTCTCGCACTTGTTCA
ACGCCAACTACAACACCGTCGTCGCCTACGACGCCGGGCCGCAATGGCAGTCGCTCCGGCGAGGTCTGCAGCACGGG
CCGCTCGGCCCCGCCCATGTTTCTGCGCAGGCTCGTTTCCACGAAGAAGACATGAAGCTCCTGGTGAGCGACATGAT
GAGAGCAGCACAGAAAGGTGGTAGCAATGGAGTGGTTGAACCTCTGGCCTATGTCCGGCGAGCCACTATCCGATTTC
TGTCTCGTCTATGCTTTGGGGAGGCCTTCAACGACGAGGCGTTCGTGGAGGGGATGGACGAAGCAGTGGAGGAGACC
ATCGGAGCCACTGGCCATGCACGCATCCTCGACGCCTTCTACTTCACTCGCCACCTCCCTATCATCCGCCGCAGCTT
CATAGATACCGTCAACGCCAAGAAGAAGATCGAGAGCCTTGTCCGGCCGTTGCTCTCCCGGCCGGCGCCACCGGGGT
CTTACCTCCACTTCCTCCTTTCCACCGACGCGCCGGAGAATATGATCATCTTTCGAATATTCGAAGTCTACTTGCTG
GGCGTGGACAGCACCGCCTCCACCACCACATGGGCTCTCGCCTTCCTCGTCTCCAACCAACAGGCGCAGGAGAAGCT
CCACAATGAGCTCGCCCAGTACTGCGCCAGCCAGAACAATCAGATCATCAAAGCAGACGACGTCGGAAAGCTGTCGT
ACCTGCTCGGGGTAGTGAAGGAGACGATGAGGATGAAGCCGATAGCGCCGCTGGCCGTCCCCCACAAGACGCTCAAG
GAGACGATGCTCGACGGAAAGAGGGTGGCGGCGGGAACGACGGTGGTAGTGAACCTCTATGCCGTCCACTACAACCC
GAAGCTATGGCCGGAGCCGGAGCAGTTCCGCCCGGAGAGGTTCGTGGTCGGCGCCAGCGGCGGCAATGGTGGGGGGT
CTTCCGAGTACATGCTGCAGTCGTACCTGCCCTTTGGAGGGGGGATGAGGTCCTGCGCAGGGATGGAGGTGGGAAAG
TTGCAGGTGGCGATGGTCGTGGCCAACCTTGTGATGGCATTTAAATGGTTGCCGGAGGAGGAGGGGAAGATGCCGGA
CCTGGCTGAAGACATGACCTTCGTGCTCATGATGAAGAAGCCATTGGCTGCCAAAATCGTTCCACGTGCATGA
cDNA Sequence Encoding for PmRDCT10 (SEQ ID NO: 16)
ATGGAGACTGAGAGGAAGTCCAGGATCTGTGTCACCGGGGCAGGAGGCTTTGTAGCCTCTTGGGTCGTCAAGCTTTT
CCTCTCCAAAGGTTATCTTGTCCATGGCACTGTCAGAGACCTCGGAGAAGAGAAGACTGCCCATTTGAGGAAGTTGG
AGGGTGCGTACCATAATCTGCAGCTGTTCAAGGCTGACTTGTTGGATTATGAGTCCTTGCTCGGGGCCATTACTGGC
TGCGATGGAGTTCTCCATGTTGCAACTCCTGTTCCTTCGAGTAAAACTGCTTATTCCGGAACTGAGTTGGTCAAGAC
TGCTGTGAATGGAACTCTGAATGTGCTCAGGGCATGTACAGAGGCAAAAGTGAAAAAGGTCATCTATGTTTCATCTA
CTGCCGCTGTTTTGGTGAATCCTAATTTACCCAAGGATAAAATCCCGGACGAAGATTGTTGGACAGACGAAGAGTAC
TGCAGGACAACTCCGTTCTTCCTGAATTGGTATTGCATCGCCAAAACAGCAGCCGAAAAGAATGCCTTGGAATATGG
AGATAAAGAAGGGATCAACGTTATATCTATTTGCCCTTCATACATCTTTGGACCTATGCTTCAACCGACAATTAATT
CAAGCAACTTGGAATTGTTGAGGCTAATGAAAGGAGATGACGAAAGCATAGAAAACAAATTTCTGCTGATGGTGGAT
GTGCGAGATGTTGCTGAAGCAATTTTACTATTATATGAGAAGCAAGAAACATCAGGGAGATACATTTCTTCGCCGCA
TGGTATGCGACAAAGCAACTTGGTTGAGAAGCTGGAGAGCCTGCAGCCGGGCTACAATTATCATAAGAACTTTGTGG
ATATTAAACCTAGTTGGACAATGATCAGCTCAGAAAAGCTCAAGAAACTTGGTTGGAAACCTAGACCACTTGAGGAC
ACTATTTCTGAAACAGTGCTGTGTTTTGAAGAGCATGGTTTGCTGGAAAATGAATAG

### REFERENCES

1. Y. N. Singh, Kava: an overview. Journal of Ethnopharmacology 37, 13-45 (1992).
2. V. Lebot, J. Lèvesque, The Origin And Distribution Of Kava (Piper Methysticum Forst. F., Piperaceae): A Phytochemical Approach. Allertonia 5, 223-281 (1989).
3. J. Sarris, E. LaPorte, I. Schweitzer, Kava: A Comprehensive Review of Efficacy, Safety, and Psychopharmacology. Australian & New Zealand Journal of Psychiatry 45, 27-35 (2011).
4. K. Shinomiya et al., Effects of kava-kava extract on the sleep-wake cycle in sleep-disturbed rats. Psychopharmacology 180, 564-569 (2005).
5. S. Cairney, P. Maruff, A. R. Clough, The neurobehavioural effects of kava. Australian and New Zealand Journal of Psychiatry 36, 807-662 (2002).
6. H. C. Chua et al., Kavain, the Major Constituent of the Anxiolytic Kava Extract, Potentiates GABAA Receptors: Functional Characteristics and Molecular Mechanism. PLoS One 11, e0157700 (2016).
7. A. Ligresti, R. Villano, M. Allara, I. Ujviry, V. Di Marzo, Kavalactones and the endocannabinoid system: The plant-derived yangonin is a novel CB1 receptor ligand. Pharmacological Research 66, 163-169 (2012).
8. L. D. Dinh et al., Interaction of various Piper methysticum cultivars with CNS receptors in vitro. Planta Med 67, 306-311 (2001).
9. N. Abu, The flavokawains: uprising medicinal chalcones. Cancer Cell Int 12, (2013).

### EQUIVALENTS AND SCOPE

In the claims articles such as “a,” “an,” and “the” may mean one or more than one unless indicated to the contrary or otherwise evident from the context. Claims or descriptions that include “or” between one or more members of a group are considered satisfied if one, more than one, or all of the group members are present in, employed in, or otherwise relevant to a given product or process unless indicated to the contrary or otherwise evident from the context. The invention includes embodiments in which exactly one member of the group is present in, employed in, or otherwise relevant to a given product or process. The invention includes embodiments in which more than one, or all of the group members are present in, employed in, or otherwise relevant to a given product or process.

Furthermore, the invention encompasses all variations, combinations, and permutations in which one or more limitations, elements, clauses, and descriptive terms from one or more of the listed claims is introduced into another claim. For example, any claim that is dependent on another claim can be modified to include one or more limitations found in any other claim that is dependent on the same base claim. Where elements are presented as lists, e.g., in Markush group format, each subgroup of the elements is also disclosed, and any element(s) can be removed from the group. It should it be understood that, in general, where the invention, or aspects of the invention, is/are referred to as comprising particular elements and/or features, certain embodiments of the invention or aspects of the invention consist, or consist essentially of, such elements and/or features. For purposes of simplicity, those embodiments have not been specifically set forth in haec verba herein. It is also noted that the terms “comprising” and “containing” are intended to be open and permits the inclusion of additional elements or steps. Where ranges are given, endpoints are included. Furthermore, unless otherwise indicated or otherwise evident from the context and understanding of one of ordinary skill in the art, values that are expressed as ranges can assume any specific value or sub-range within the stated ranges in different embodiments of the invention, to the tenth of the unit of the lower limit of the range, unless the context clearly dictates otherwise.

This application refers to various issued patents, published patent applications, journal articles, and other publications, all of which are incorporated herein by reference. If there is a conflict between any of the incorporated references and the instant specification, the specification shall control. In addition, any particular embodiment of the present invention that falls within the prior art may be explicitly excluded from any one or more of the claims. Because such embodiments are deemed to be known to one of ordinary skill in the art, they may be excluded even if the exclusion is not set forth explicitly herein. Any particular embodiment of the invention can be excluded from any claim, for any reason, whether or not related to the existence of prior art.

Those skilled in the art will recognize or be able to ascertain using no more than routine experimentation many equivalents to the specific embodiments described herein. The scope of the present embodiments described herein is not intended to be limited to the above Description, but rather is as set forth in the appended claims. Those of ordinary skill in the art will appreciate that various changes and modifications to this description may be made without departing from the spirit or scope of the present invention, as defined in the following claims.

1. A method for producing a compound of Formula (II), the method comprising:
condensing a compound of Formula (I), or a salt thereof, with coenzyme A (CoA) using an enzyme that is at least 80% identical to Pm4CL1 (SEQ ID NO: 1) to produce a compound of Formula (II);
wherein: 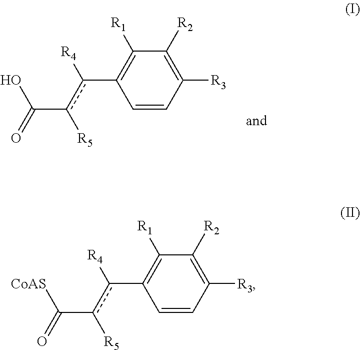 [symbol]
is a single bond or a double bond;
each of R₁, R₂, and R₃ independently is hydrogen, optionally substituted, cyclic or acyclic aliphatic, or ORₓ, wherein Rₓ is hydrogen or optionally substituted, cyclic or acyclic aliphatic; and
each of R₄ and R₅ independently is hydrogen or optionally substituted, cyclic or acyclic aliphatic.

2. The method of claim 1, wherein [symbol] is a single bond.

3. The method of claim 1, wherein [symbol] is a double bond

4. The method of claim 1, wherein R₁ is selected from the group consisting of hydrogen, —OH, and —OCH₃.

5. The method of claim 1, wherein R₂ is selected from the group consisting of hydrogen, —OH, and —OCH₃.

6. The method of claim 1, wherein R₃ is selected from the group consisting of hydrogen, —OH, and —OCH₃.

7. The method of claim 1, wherein both R₄ and R₅ are hydrogen.

8. The method of claim 1, wherein R₁, R₂, and R₃ are hydrogen.

9. The method of claim 1, wherein R₁, R₂, and R₃ are —OH.

10. The method of claim 1, wherein R₁ and R₃ are —OH.

11. The method of claim 1, wherein R₂ and R₃ are —OH.

12. The method of claim 1, wherein R₂ is —OCH₃.

13. (canceled)

14. The method of claim 1, wherein the enzyme is a purified or partially purified enzyme

15. (canceled)

16. The method of claim 1, wherein the enzyme is produced by a recombinant cell line.

17-19. (canceled)

20. The method of claim 1, wherein the enzyme is produced by a recombinant non-human organism is-selected from the group consisting of bacteria, yeast, and plant.

21. (canceled)

22. The method of claim 1, wherein the method is conducted in a recombinant cell.

23. (canceled)

24. The method of claim 22, wherein the recombinant cell is an Escherichia coli cell.

25. (canceled)

26. The method of claim 22, wherein the recombinant cell is a Saccharomyces cell.

27. (canceled)

28. The method of claim 22, wherein the recombinant cell is a Nicotiana cell.

29. (canceled)

30. The method of claim 22, wherein the enzyme is heterologous to the recombinant cell.

31-289. (canceled)
